SEQUENCE LISTING (124 sequences)

SEQ ID NO: 1 - protein, 221 aa, synthetic construct
MQRLIEDICLPRWGCLWEDDFGGGGSGGGGSGGGGSLRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLKCRIIHNNNC

SEQ ID NO: 2 - protein, 226 aa, synthetic construct
MQRLIEDICLPRWGCLWEDDFGGGGSGGGGSGGGGSGGGGSLRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLKCRIIHNNNC

SEQ ID NO: 3 - protein, 221 aa, synthetic construct
MQRLIEDICLPRWGCLWEDDFEAAAKEAAAKEAAAKLRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLKCRIIHNNNC

SEQ ID NO: 4 - protein, 218 aa, synthetic construct
MQRLIEDICLPRWGCLWEDDFAEAAAKEAAAKALRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLKCRIIHNNNC

SEQ ID NO: 5 - protein, 223 aa, synthetic construct
MQRLIEDICLPRWGCLWEDDFAEAAAKEAAAKEAAAKALRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLKCRIIHNNNC

SEQ ID NO: 6 - protein, 228 aa, synthetic construct
MQRLIEDICLPRWGCLWEDDFAEAAAKEAAAKEAAAKEAAAKALRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLKCRIIHNNNC

SEQ ID NO: 7 - protein, 233 aa, synthetic construct
MQRLIEDICLPRWGCLWEDDFAEAAAKEAAAKEAAAKEAAAKEAAAKALRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLKCRIIHNNNC

SEQ ID NO: 8 - protein, 250 aa, synthetic construct
MQRLIEDICLPRWGCLWEDDFAEAAAKEAAAKEAAAKEAAAKAAEAAAKEAAAKEAAAKEAAAKALRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLKCRIIHNNNC

SEQ ID NO: 9 - protein, 252 aa, synthetic construct
MQRLIEDICLPRWGCLWEDDFAEAAAKEAAAKEAAAKEAAAKALEAEAAAKEAAAKEAAAKEAAAKALRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLKCRIIHNNNC

SEQ ID NO: 10 - protein, 222 aa, synthetic construct
MQRLIEDICLPRWGCLWEDDFEAAAKEAAAKEAAAKLSRLFDHAMLQAHRAHQLAIDTYQEFEETYIPKDQKYSFLHDSQTSFCFSDSIPTPSNMEETQQKSNLELLRISLLLIESWLEPVRFLRSMFANNLVYDTSDSDDYHLLKDLEERIQTLMGRLEDGSRRTGQILKQTYSKFDTNSHNHDALLKNYGLLYCFRKDMDKVETFLRMVQCRSVEGSCGF

SEQ ID NO: 11 - protein, 222 aa, synthetic construct
MQRLIEDICLPRWGCLWEDDFEAAAKEAAAKEAAAKLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEERIQTLMGRLEDGSPRTGQIFKQTYSKFDTNSHNDDALLKNYGLLYCFRKDMDKVETFLRIVQCRSVEGSCGF

SEQ ID NO: 12 - protein, 11 aa, synthetic construct
  VARIANT 2: note = Any amino acid
  VARIANT 6: note = Any amino acid
DXCLPXWGCLW

SEQ ID NO: 13 - protein, 11 aa, synthetic construct
DICLPRWGCLW

SEQ ID NO: 14 - protein, 21 aa, synthetic construct
MQRLIEDICLPRWGCLWEDDF

SEQ ID NO: 15 - protein, 48 aa, synthetic construct
  VARIANT 4: note = E, S, Q, or C
  VARIANT 7: note = E, S, or C
  VARIANT 8: note = A or S
  VARIANT 11: note = A, S, or R
  VARIANT 15: note = A, S, C, or K
  VARIANT 27: note = D or E
  VARIANT 40: note = D or E
  VARIANT 41: note = A or E
  VARIANT 44: note = A or K
  VARIANT 45: note = A, S, or E
  VARIANT 46: note = May be absent
  VARIANT 47: note = May be absent
SLAXAKXXANXELDXYGVSDFYKRLIXKAKTVEGVEALKXXILXXLPK

SEQ ID NO: 16 - protein, 50 aa, synthetic construct
  VARIANT 1: note = Any amino acid
  VARIANT 2: note = Any amino acid
  VARIANT 3: note = Any amino acid
  VARIANT 4: note = Any amino acid
  VARIANT 5: note = Any amino acid
  VARIANT 26: note = Any amino acid
  VARIANT 27: note = Any amino acid
  VARIANT 35: note = Any amino acid
  VARIANT 36: note = Any amino acid
  VARIANT 42: note = Any amino acid
  VARIANT 46: note = Any amino acid
XXXXXLKEAKEKAIEELKKAGITSDXXFDLINKAXXVEGVNXLKDXILKA

SEQ ID NO: 17 - protein, 20 aa, synthetic construct
QRLIEDICLPRWGCLWEDDF

SEQ ID NO: 18 - protein, 34 aa, synthetic construct
DTAVYYCNAAASYSDYDVFGGGTDFGPWGQGTQV

SEQ ID NO: 19 - protein, 18 aa, synthetic construct
AARYWDFDVFGGGTPVGG

SEQ ID NO: 20 - protein, 18 aa, synthetic construct
AARDWDFDVFGGGTPVGG

SEQ ID NO: 21 - protein, 18 aa, synthetic construct
AARSWDFDVFGGGTPVGG

SEQ ID NO: 22 - protein, 18 aa, synthetic construct
YWWERRDWDFDVFGGGTP

SEQ ID NO: 23 - protein, 18 aa, synthetic construct
WWEQDRDWDFDVFGGGTP

SEQ ID NO: 24 - protein, 46 aa, synthetic construct
WWEQDRDWDFDVFGGGTPGGGGGGGGGSWWEQDRDWDFDVFGGGTP

SEQ ID NO: 25 - protein, 46 aa, Streptococcus dysgalactiae
LAEAKVLANRELDKYGVSDYYKNLINNAKTVEGVKALIDEILAALP

SEQ ID NO: 26 - protein, 53 aa, Finegoldia magna
TIDQWLLKNAKEDAIAELKKAGITSDFYFNAINKAKTVEEVNALKNEILKAHA

SEQ ID NO: 27 - protein, 52 aa, Streptococcus equi
DITGAALLEAKEAAINELKQYGISDYYVTLINKAKTVEGVNALKAEILSALP

SEQ ID NO: 28 - protein, 52 aa, Streptococcus dysgalactiae
ALKAAALAKAKADAIEILKKYGIGDYYIKLINNGKTAEGVTALKDEILASLP

SEQ ID NO: 29 - protein, 46 aa, synthetic construct
LAQAKEAAIKELKQYGIGDYYIKLINNAKTVEGVESLKNEILKALP

SEQ ID NO: 30 - protein, 45 aa, synthetic construct
LKEAKEKAIEELKKAGITSDYYFDLINKAKTVEGVNALKDEILKA

SEQ ID NO: 31 - protein, 46 aa, synthetic construct
LAEAKVLANRELDKYGVSDYYKNIINRAKTVEGVRALKLHILAALP

SEQ ID NO: 32 - protein, 46 aa, synthetic construct
LAEAKVLANRELDKYGVSDYYKNLINKAKTVEGVEALTLHILAALP

SEQ ID NO: 33 - protein, 46 aa, synthetic construct
LAEAKVLANRELDKYGVSDFYKRLINKAKTVEGVEALKLHILAALP

SEQ ID NO: 34 - protein, 46 aa, synthetic construct
LAEAKEDAIKELDKYGVSDYYKRLISKAKTVEGVKALISEILAALP

SEQ ID NO: 35 - protein, 46 aa, synthetic construct
LAEAKEDAIKELDKYGVSDYYKNLINKAKTVEGVEALTLHILAALP

SEQ ID NO: 36 - protein, 46 aa, synthetic construct
LAEAKEDAIKELDKYGVSDYYKNLINKAKTVEGVEALISEILAALP

SEQ ID NO: 37 - protein, 46 aa, synthetic construct
LAEAKEAANAELDSYGVSDFYKRLIDKAKTVEGVEALKDAILAALP

SEQ ID NO: 38 - protein, 52 aa, synthetic construct
DITGAALLEAKDAAINELKQYGISDYYVHLINKADTVEGVNALKDEILSALP

SEQ ID NO: 39 - protein, 64 aa, synthetic construct
VKVKFVFSGEEKEVDTSKIKWVLRWGKAVGFKYDDNGKIGYGFVAEKDAPKELLDMLARAEREK

SEQ ID NO: 40 - protein, 64 aa, synthetic construct
VKVKFWPRGEEKVVDTSKIAWVLRADKTVMFKYDDNGKKGYGVVLEKDAPKELLDMLARAEREK

SEQ ID NO: 41 - protein, 64 aa, synthetic construct
VKVKFVFSGEEKEVETSKIKWVLRWGKAVGFKYDDQGKIGYGFVAEKDAPKELLDLLARAEREK

SEQ ID NO: 42 - protein, 64 aa, synthetic construct
VKVKFWPRGEEKVVETSKIAWVLRADKTVMFKYDDQGKKGYGVVLEKDAPKELLDLLARAEREK

SEQ ID NO: 43 - protein, 123 aa, synthetic construct
QVQLVESGGGLVQPGGSLRLSCAASGFTFSNYWMYWVRVAPGKGLERISRDISTGGGYSYYADSVKGRFTISRDNAKNTLYLQMNSLKPEDTALYYCAKDREAQVDTLDFDYRGQGTQVTVSS

SEQ ID NO: 44 - protein, 115 aa, synthetic construct [sequence omitted]

SEQ ID NO: 45 - protein, 115 aa, synthetic construct [sequence omitted]

SEQ ID NO: 46 - protein, 115 aa, synthetic construct
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSS

SEQ ID NO: 47 - protein, 185 aa, synthetic construct
LRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLKCRIIHNNNC

SEQ ID NO: 48 - protein, 185 aa, synthetic construct
LRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEAILSKAVEIEEQTKRLLEKMELIVSQVHPETKENEIYPVWSGLPSLQMADEESRLSAYYNLLHCLRRDSHKIDNYLKLLKCRIIHNNNC

SEQ ID NO: 49 - protein, 185 aa, synthetic construct
LRDLFDRAVVLSHYIHNLSSEMFSEFDKRYTHGRGFITKAINSCHTASLATPENKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 50 - protein, 185 aa, synthetic construct
LRDLFDRAVVQSHYIHNLSSEMFSEFDKRYTRGRGFIAKAFNSCHTSSLATPENKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 51 - protein, 185 aa, synthetic construct
LRDLFDRAVVLSHYIHNLSSEMFSEFDKRYTHGRGFITKAINSCHTSSLATPSNKEEAQQMNQKDFLSLIVSILRSWNEPLYHLVTEVRGMQEAPEAILSKAVEIEEQTKRLLEGMELIVSQVHPETKENEIYPVWSGLPSLQMADEESRLSAYYNLLHCLRRDSDKIATFLRLLKCRIIHNNNC

SEQ ID NO: 52 - protein, 186 aa, synthetic construct
LSRLFDHAMLQAHRAHQLAIDTYQEFEETYIPKDQKYSFLHDSQTSFCFSDSIPTPSNMEETQQKSNLELLRISLLLIESWLEPVRFLRSMFANNLVYDTSDSDDYHLLKDLEERIQTLMGRLEDGSRRTGQILKQTYSKFDTNSHNHDALLKNYGLLYCFRKDMDKVETFLRMVQCRSVEGSCGF

SEQ ID NO: 53 - protein, 186 aa, synthetic construct
LSRLFDHAMLQAHRAHQLAIDTYQEFEETYIPKDQKYSFLHDSQTSFCFSESIPTPSNREETQQKSNLELLRISLLLIESWLEPVRFLRSMFANNLVYDTSDSDDYHLLKDLEERIQTLMGRLEDGSRRTGQILKQTYSKFDTNSHNHDALLKNYGLLYCFRKDMDKVATFLRIVQCRSVEGSCGF

SEQ ID NO: 54 - protein, 186 aa, synthetic construct
LSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEERIQTLMGRLEDGSPRTGQIFKQTYSKFDTNSHNDDALLKNYGLLYCFRKDMDKVETFLRIVQCRSVEGSCGF

SEQ ID NO: 55 - protein, 186 aa, synthetic construct
LSRLFDNAMLRADRLNQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEKIQTLMGRLEDGSPRTGQIFKQTYSKFDTNSHNDDALLKNYGLLYCFNADMSRVSTFLRTVQCRSVEGSCGF

SEQ ID NO: 56 - protein, 5 aa, synthetic construct
EAAAK

SEQ ID NO: 57 - protein, 10 aa, synthetic construct
EAAAKEAAAK

SEQ ID NO: 58 - protein, 15 aa, synthetic construct
EAAAKEAAAKEAAAK

SEQ ID NO: 59 - protein, 20 aa, synthetic construct
EAAAKEAAAKEAAAKEAAAK

SEQ ID NO: 60 - protein, 25 aa, synthetic construct
EAAAKEAAAKEAAAKEAAAKEAAAK

SEQ ID NO: 61 - protein, 30 aa, synthetic construct
EAAAKEAAAKEAAAKEAAAKEAAAKEAAAK

SEQ ID NO: 62 - protein, 35 aa, synthetic construct
EAAAKEAAAKEAAAKEAAAKEAAAKEAAAKEAAAK

SEQ ID NO: 63 - protein, 40 aa, synthetic construct
EAAAKEAAAKEAAAKEAAAKEAAAKEAAAKEAAAKEAAAK

SEQ ID NO: 64 - protein, 45 aa, synthetic construct
EAAAKEAAAKEAAAKEAAAKEAAAKEAAAKEAAAKEAAAKEAAAK

SEQ ID NO: 65 - protein, 50 aa, synthetic construct
EAAAKEAAAKEAAAKEAAAKEAAAKEAAAKEAAAKEAAAKEAAAKEAAAK

SEQ ID NO: 66 - protein, 6 aa, synthetic construct
AEAAKA

SEQ ID NO: 67 - protein, 12 aa, synthetic construct
AEAAAKEAAAKA

SEQ ID NO: 68 - protein, 17 aa, synthetic construct
AEAAAKEAAAKEAAAKA

SEQ ID NO: 69 - protein, 22 aa, synthetic construct
AEAAAKEAAAKEAAAKEAAAKA

SEQ ID NO: 70 - protein, 27 aa, synthetic construct
AEAAAKEAAAKEAAAKEAAAKEAAAKA

SEQ ID NO: 71 - protein, 32 aa, synthetic construct
AEAAAKEAAAKEAAAKEAAAKEAAAKEAAAKA

SEQ ID NO: 72 - protein, 37 aa, synthetic construct
AEAAAKEAAAKEAAAKEAAAKEAAAKEAAAKEAAAKA

SEQ ID NO: 73 - protein, 42 aa, synthetic construct
AEAAAKEAAAKEAAAKEAAAKEAAAKEAAAKEAAAKEAAAKA

SEQ ID NO: 74 - protein, 47 aa, synthetic construct
AEAAAKEAAAKEAAAKEAAAKEAAAKEAAAKEAAAKEAAAKEAAAKA

SEQ ID NO: 75 - protein, 52 aa, synthetic construct
AEAAAKEAAAKEAAAKEAAAKEAAAKEAAAKEAAAKEAAAKEAAAKEAAAKA

SEQ ID NO: 76 - protein, 44 aa, synthetic construct
AEAAAKEAAAKEAAAKEAAAKAAEAAAKEAAAKEAAAKEAAAKA

SEQ ID NO: 77 - protein, 46 aa, synthetic construct
AEAAAKEAAAKEAAAKEAAAKALEAEAAAKEAAAKEAAAKEAAAKA

SEQ ID NO: 78-80 - skipped (3 entries)

SEQ ID NO: 81 - protein, 8 aa, synthetic construct
  VARIANT 1: note = Any amino acid, preferably selected from alanine, lysine, or glutamine
  VARIANT 3: note = Any amino acid, preferably selected from alanine, lysine, or glutamine
  VARIANT 5: note = Any amino acid, preferably selected from alanine, lysine, or glutamine
  VARIANT 7: note = Any amino acid, preferably selected from alanine, lysine, or glutamine
XPXPXPXP

SEQ ID NO: 82 - protein, 10 aa, synthetic construct
  VARIANT 1: note = Any amino acid, preferably selected from alanine, lysine, or glutamine
  VARIANT 3: note = Any amino acid, preferably selected from alanine, lysine, or glutamine
  VARIANT 5: note = Any amino acid, preferably selected from alanine, lysine, or glutamine
  VARIANT 7: note = Any amino acid, preferably selected from alanine, lysine, or glutamine
  VARIANT 9: note = Any amino acid, preferably selected from alanine, lysine, or glutamine
XPXPXPXPXP

SEQ ID NO: 83 - protein, 12 aa, synthetic construct
  VARIANT 1: note = Any amino acid, preferably selected from alanine, lysine, or glutamine
  VARIANT 3: note = Any amino acid, preferably selected from alanine, lysine, or glutamine
  VARIANT 5: note = Any amino acid, preferably selected from alanine, lysine, or glutamine
  VARIANT 7: note = Any amino acid, preferably selected from alanine, lysine, or glutamine
  VARIANT 9: note = Any amino acid, preferably selected from alanine, lysine, or glutamine
  VARIANT 11: note = Any amino acid, preferably selected from alanine, lysine, or glutamine
XPXPXPXPXPXP

SEQ ID NO: 84 - protein, 14 aa, synthetic construct
  VARIANT 1: note = Any amino acid, preferably selected from alanine, lysine, or glutamine
  VARIANT 3: note = Any amino acid, preferably selected from alanine, lysine, or glutamine
  VARIANT 5: note = Any amino acid, preferably selected from alanine, lysine, or glutamine
  VARIANT 7: note = Any amino acid, preferably selected from alanine, lysine, or glutamine
  VARIANT 9: note = Any amino acid, preferably selected from alanine, lysine, or glutamine
  VARIANT 11: note = Any amino acid, preferably selected from alanine, lysine, or glutamine
  VARIANT 13: note = Any amino acid, preferably selected from alanine, lysine, or glutamine
XPXPXPXPXPXPXP

SEQ ID NO: 85 - protein, 16 aa, synthetic construct
  VARIANT 1: note = Any amino acid, preferably selected from alanine, lysine, or glutamine
  VARIANT 3: note = Any amino acid, preferably selected from alanine, lysine, or glutamine
  VARIANT 5: note = Any amino acid, preferably selected from alanine, lysine, or glutamine
  VARIANT 7: note = Any amino acid, preferably selected from alanine, lysine, or glutamine
  VARIANT 9: note = Any amino acid, preferably selected from alanine, lysine, or glutamine
  VARIANT 11: note = Any amino acid, preferably selected from alanine, lysine, or glutamine
  VARIANT 13: note = Any amino acid, preferably selected from alanine, lysine, or glutamine
  VARIANT 15: note = Any amino acid, preferably selected from alanine, lysine, or glutamine
XPXPXPXPXPXPXPXP

SEQ ID NO: 86 - protein, 18 aa, synthetic construct
  VARIANT 1: note = Any amino acid, preferably selected from alanine, lysine, or glutamine
  VARIANT 3: note = Any amino acid, preferably selected from alanine, lysine, or glutamine
  VARIANT 5: note = Any amino acid, preferably selected from alanine, lysine, or glutamine
  VARIANT 7: note = Any amino acid, preferably selected from alanine, lysine, or glutamine
  VARIANT 9: note = Any amino acid, preferably selected from alanine, lysine, or glutamine
  VARIANT 11: note = Any amino acid, preferably selected from alanine, lysine, or glutamine
  VARIANT 13: note = Any amino acid, preferably selected from alanine, lysine, or glutamine
  VARIANT 15: note = Any amino acid, preferably selected from alanine, lysine, or glutamine
  VARIANT 17: note = Any amino acid, preferably selected from alanine, lysine, or glutamine
XPXPXPXPXPXPXPXPXP

SEQ ID NO: 87 - protein, 20 aa, synthetic construct
  VARIANT 1: note = Any amino acid, preferably selected from alanine, lysine, or glutamine
  VARIANT 3: note = Any amino acid, preferably selected from alanine, lysine, or glutamine
  VARIANT 5: note = Any amino acid, preferably selected from alanine, lysine, or glutamine
  VARIANT 7: note = Any amino acid, preferably selected from alanine, lysine, or glutamine
  VARIANT 9: note = Any amino acid, preferably selected from alanine, lysine, or glutamine
  VARIANT 11: note = Any amino acid, preferably selected from alanine, lysine, or glutamine
  VARIANT 13: note = Any amino acid, preferably selected from alanine, lysine, or glutamine
  VARIANT 15: note = Any amino acid, preferably selected from alanine, lysine, or glutamine
  VARIANT 17: note = Any amino acid, preferably selected from alanine, lysine, or glutamine
  VARIANT 19: note = Any amino acid, preferably selected from alanine, lysine, or glutamine
XPXPXPXPXPXPXPXPXPXP

SEQ ID NO: 88 - protein, 15 aa, synthetic construct
GGGGSGGGGSGGGGS

SEQ ID NO: 89 - protein, 20 aa, synthetic construct
GGGGSGGGGSGGGGSGGGGS

SEQ ID NO: 90 - skipped

SEQ ID NO: 91 - protein, 4 aa, synthetic construct
GSGS

SEQ ID NO: 92 - protein, 6 aa, synthetic construct
GSGSGS

SEQ ID NO: 93 - protein, 8 aa, synthetic construct
GSGSGSGS

SEQ ID NO: 94 - protein, 10 aa, synthetic construct
GSGSGSGSGS

SEQ ID NO: 95 - protein, 12 aa, synthetic construct
GSGSGSGSGSGS

SEQ ID NO: 96 - protein, 14 aa, synthetic construct
GSGSGSGSGSGSGS

SEQ ID NO: 97 - protein, 14 aa, synthetic construct
GSGSGSGSGSGSGS

SEQ ID NO: 98 - protein, 16 aa, synthetic construct
GSGSGSGSGSGSGSGS

SEQ ID NO: 99 - protein, 16 aa, synthetic construct
GSGSGSGSGSGSGSGS

SEQ ID NO: 100 - protein, 5 aa, synthetic construct
GSGGS

SEQ ID NO: 101 - protein, 10 aa, synthetic construct
GSGGSGSGGS

SEQ ID NO: 102 - protein, 15 aa, synthetic construct
GSGGSGSGGSGSGGS

SEQ ID NO: 103 - protein, 20 aa, synthetic construct
GSGGSGSGGSGSGGSGSGGS

SEQ ID NO: 104 - protein, 25 aa, synthetic construct
GSGGSGSGGSGSGGSGSGGSGSGGS

SEQ ID NO: 105 - protein, 4 aa, synthetic construct
GGGS

SEQ ID NO: 106 - protein, 8 aa, synthetic construct
GGGSGGGS

SEQ ID NO: 107 - protein, 12 aa, synthetic construct
GGGSGGGSGGGS

SEQ ID NO: 108 - protein, 16 aa, synthetic construct
GGGSGGGSGGGSGGGS

SEQ ID NO: 109 - protein, 20 aa, synthetic construct
GGGSGGGSGGGSGGGSGGGS

SEQ ID NO: 110 - DNA, 666 bp, synthetic construct
  source 1..666: mol_type = other DNA
atgcaacgtcttatcgaggacatttgcttacctcggtggggctgcttatgggaggatgattttggcggcggtgggtccggtggtggtggcagtggtggtggcgggagcttacgcgacctctttgaccgcgcggtcgtgctgagccactacattcataatctgagttccgagatgtttagcgaatttgataagcgctacacgcatggtcgcgggtttattacgaaggccattaattcatgtcacacgagttccttggcgacgccagaggacaaggaacaagcacagcaaatgaatcaaaaggactttttatccctgatcgtgtccattctgcggagttggaacgaaccgctttaccaccttgtgaccgaagtccgtggtatgcaggaagctcctgaggcgatcctctccaaggcagtcgaaattgaggaacaaaccaaacggttgttggaacggatggagctgatcgtatcacaagtgcatcctgagactaaagaaaacgaaatctaccctgtttggtcgggccttccatcattacaaatggctgacgaagagtcacgccttagcgcgtactacaacttactgcactgccttcggcgcgattcccataaaatcgacaactatctcaaactgctcaaatgccggatcatccataacaataattgttaa

SEQ ID NO: 111 - DNA, 681 bp, synthetic construct
  source 1..681: mol_type = other DNA
atgcagcgtttaattgaggatatttgtctgcctcgttggggctgtttgtgggaagatgacttcggtggcggtggctccggggggggtggtagcggtgggggtggctccggtggtggtgggagtttacgggacttatttgatcgggctgtcgtgctgtctcactatatccataatttaagttccgagatgttttcggaatttgacaaacgctacacgcacggtcgcggtttcattacaaaggccattaatagctgccacacatcgtcgttagccacgcctgaggataaggaacaagcccagcaaatgaaccaaaaggactttctgagtcttatcgtatcgatccttcggtcttggaacgaaccgttgtaccacctcgtgacggaggttcgtggtatgcaagaagctccggaagccattctctccaaggctgttgagatcgaagaacagacgaaacgtctcttggaacgtatggagctgatcgttagtcaagtacatcctgagacaaaggagaatgaaatttatccagtttggtcgggccttccttcgctccagatggctgatgaggaaagccgcctctcggcctattataatcttcttcactgtttgcgccgcgattcacataaaatcgacaattacttaaaactcctgaagtgtcggatcattcataacaacaactgttaa

SEQ ID NO: 112 - DNA, 666 bp, synthetic construct
  source 1..666: mol_type = other DNA
atgcagcgtctcattgaagatatttgtctgccgcgctggggctgcctctgggaagatgattttgaggctgccgccaaggaggccgcagccaaagaggctgccgctaagctgcgggacttgtttgaccgggcggttgtattgagtcactacattcataacttatcctccgaaatgttttctgagttcgataaacggtacacgcatggccgggggtttatcacaaaggccatcaactcatgccataccagtagcttggcgacaccggaggataaagagcaggctcaacagatgaaccagaaggacttcctgagccttattgtttccatcttgcggtcttggaatgaaccattatatcacttagtcaccgaggtgcgtgggatgcaagaagcgcctgaggcgattctcagtaaggccgtggaaatcgaagaacagaccaagcgtctgcttgaacggatggaacttattgtttcgcaagtccacccggagactaaggaaaacgaaatttatcctgtctggtcgggtcttccatctttacaaatggcagacgaagagtcgcggctttcggcgtattataacctcttgcattgcttacggcgggatagtcataagattgacaactacctcaaattacttaagtgccggattatccacaacaataactgttaa

SEQ ID NO: 113 - DNA, 657 bp, synthetic construct
  source 1..657: mol_type = other DNA
atgcagcgtcttattgaggatatctgtttacctcggtggggctgtctctgggaggatgactttgccgaagctgctgctaaagaagccgccgcaaaggcccttcgggatctgtttgaccgggcagttgtcttgagtcactacattcataatttaagcagtgagatgttctcagagttcgataagcgctatactcacggccgtggttttattactaaggcgattaattcatgtcacacgtcaagtctggctacacctgaggataaagagcaagcccagcagatgaatcagaaggactttttaagtctgattgtgtcaatcctgcgctcttggaatgaaccgctgtatcatttggtcactgaagtccgcgggatgcaagaagcacctgaggctatccttagtaaggccgttgaaatcgaagaacagacgaagcgtcttctggaacgcatggaactgattgttagccaagttcatcctgaaactaaagaaaatgagatttatcctgtttggtccggcctcccaagtcttcagatggcggatgaggaatcgcgtctttccgcatactataatctgcttcattgcttgcgccgcgattcacataagattgacaactatcttaaacttcttaaatgtcggattatccacaacaacaactgctaa

SEQ ID NO: 114 - DNA, 672 bp, synthetic construct
  source 1..672: mol_type = other DNA
atgcaacgcttaattgaggatatctgcctccctcggtggggttgcctgtgggaagacgactttgcagaagcagcggcgaaagaagctgccgcaaaagaggccgccgccaaggccctccgtgacttattcgaccgtgcagtagtgttgagccactatatccataacttgtcttcggaaatgttctcagagtttgacaagcgttacacccatgggcgcggttttatcacgaaggcaattaactcgtgccacacatctagtttagccactcctgaggataaagaacaagcacaacaaatgaaccaaaaagacttcttaagtctcatcgtctcgatccttcgctcatggaacgaacctctctatcatctggtgacggaggttcgcggtatgcaggaagctccagaagcaattctgtcaaaagcggtcgagatcgaagaacaaaccaaacggttgttagaacgcatggagcttatcgtctcgcaggtgcatccagaaacaaaggaaaatgaaatttatccggtttggtctggtttgccttcactccagatggctgacgaggaatcccgtttatccgcctactacaatcttctccattgcctccgtcgtgattcccataaaattgacaactatttgaaattgctcaagtgccgcattatccacaacaataactgttga

SEQ ID NO: 115 - DNA, 687 bp, synthetic construct
  source 1..687: mol_type = other DNA
atgcaacgcttaattgaggatatctgcctccctcggtggggttgcctgtgggaagacgactttgcagaagccgccgctaaggaagcagcggcgaaagaagctgccgcaaaagaggccgccgccaaggccctccgtgacttattcgaccgtgcagtagtgttgagccactatatccataacttgtcttcggaaatgttctcagagtttgacaagcgttacacccatgggcgcggttttatcacgaaggcaattaactcgtgccacacatctagtttagccactcctgaggataaagaacaagcacaacaaatgaaccaaaaagacttcttaagtctcatcgtctcgatccttcgctcatggaacgaacctctctatcatctggtgacggaggttcgcggtatgcaggaagctccagaagcaattctgtcaaaagcggtcgagatcgaagaacaaaccaaacggttgttagaacgcatggagcttatcgtctcgcaggtgcatccagaaacaaaggaaaatgaaatttatccggtttggtctggtttgccttcactccagatggctgacgaggaatcccgtttatccgcctactacaatcttctccattgcctccgtcgtgattcccataaaattgacaactatttgaaattgctcaagtgccgcattatccacaacaataactgttga

SEQ ID NO: 116 - DNA, 702 bp, synthetic construct
  source 1..702: mol_type = other DNA
atgcagcgccttattgaggacatttgtttaccgcgttggggttgcctgtgggaagacgacttcgcagaagctgctgccaaagaggcggcagctaaggaggccgccgctaaagaagcagcagctaaagaggcagctgcaaaagccttgcgcgatttatttgatcgcgccgtggtcctgtcccactatattcataacttatcatcagaaatgttcagtgagttcgataaacggtacacacatgggcggggcttcattacgaaggccattaactcatgtcatacatcaagtctggcaacaccagaggataaagaacaggctcaacagatgaaccagaaagacttcctttcattgattgtcagtatcttacggtcgtggaatgaacctctgtaccatttggtcactgaagtccgtgggatgcaggaggccccggaggcaattttgtcgaaagcggtggagatcgaagaacagactaaacggttgctcgaacgcatggaactgatcgtatctcaggttcatcctgaaactaaggaaaatgagatctaccctgtctggagtgggttaccgtcgcttcaaatggcggatgaggagtcgcgtttgtctgcatattacaacctccttcactgtctccggcgcgactctcacaaaatcgataattatctcaagttactcaagtgccgcattattcacaataataactgttaa

SEQ ID NO: 117 - DNA, 753 bp, synthetic construct
  source 1..753: mol_type = other DNA
atgcagcgcctgatcgaggatatctgtcttcctcgttgggggtgtctgtgggaggacgactttgcggaagcagcggcaaaggaagccgctgccaaagaagccgcagcgaaagaggccgccgcgaaagctgccgaggccgctgccaaggaggctgcggcgaaggaagctgctgccaaggaagcggcggcgaaagcattacgtgatttgtttgatcgggcagttgtcctgagccattacatccacaatttgtcctcagaaatgttttcggagtttgacaagcgctatacccatgggcggggcttcatcaccaaagcaatcaactcctgccatacttcttctcttgccacacctgaagataaagaacaagctcaacagatgaatcagaaagactttttgtcgctcattgtgagtattctgcggtcttggaatgaaccgctctaccaccttgttacggaggtccgtgggatgcaagaagcgccggaggcgattctcagcaaagccgtggagattgaagagcagacgaagcgcctccttgagcgtatggaactcattgtgagtcaagtacacccggaaacaaaggagaatgaaatctaccctgtctggtcaggcttaccttctttacagatggcagatgaagagtcacgtctctctgcctattacaacttactgcattgcttacggcgtgatagtcataaaatcgacaattaccttaaactgttaaagtgtcgcattatccataataataattgctga

SEQ ID NO: 118 - DNA, 759 bp, synthetic construct
  source 1..759: mol_type = other DNA
atgcaacgcttaatcgaagatatttgtctcccgcgctgggggtgcctgtgggaggacgattttgctgaggctgcagcgaaggaggctgcggctaaagaggcagccgcaaaagaagcagcggcaaaagctttagaggcagaggctgccgcgaaggaagccgcagcaaaagaggcggcggccaaggaggcagccgctaaggccctgcgtgaccttttcgatcgtgcagtagtgttatcgcactatatccataacttgtcctccgaaatgtttagcgaatttgataaacggtacacgcacgggcgcgggtttatcacgaaagcgattaactcctgccatacaagcagtctggccacaccggaagataaggaacaggcacaacaaatgaaccaaaaggacttcttgtctctgattgtttcgattttacggtcttggaacgagccgctttaccatctcgtaaccgaagtccgcgggatgcaggaggccccagaagcgatcttaagtaaggcagttgagattgaggaacaaactaaacggttactcgaacggatggagctgatcgtcagtcaagttcatccagaaactaaggaaaacgagatttatccagtatggagtgggttaccaagcctccaaatggctgatgaggagagtcgtttatcagcttactacaacttgcttcattgcctgcgccgtgactcacacaaaattgataattaccttaagctcctcaagtgtcgtatcatccataataataactgctga

SEQ ID NO: 119 - DNA, 669 bp, synthetic construct
  source 1..669: mol_type = other DNA
atgcagcgtctgattgaggacatttgccttccacgctggggctgtttatgggaggacgattttgaggctgccgccaaggaggccgcagccaaagaggctgccgctaagctgtcccgcctctttgaccacgcaatgctccaagctcaccgggctcatcaattggcgatcgacacgtaccaggaatttgaagaaacatacattccgaaagatcagaaatatagttttctgcatgactcgcagacttcgttttgctttagcgacagtatcccaacgccatcgaacatggaggagacgcaacaaaagtcaaacttagagttactgcgtattagcctcttgttgattgagtcctggctcgaaccagtacggtttttacggagtatgtttgcaaataatctcgtctacgacactagtgatagtgacgactatcatttactcaaggatcttgaagagcggattcagaccctcatgggtcgtcttgaagatgggtcccgccggaccggtcaaatcctgaaacaaacctactccaagttcgacacaaacagccataatcacgacgccttattaaaaaactatggcttactgtattgcttccgcaaggacatggacaaagtggagacattcttgcgtatggtgcaatgtcgctcggtggagggcagttgcggtttttga

SEQ ID NO: 120 - DNA, 669 bp, synthetic construct
  source 1..669: mol_type = other DNA
atgcaacgcctgatcgaagacatctgtttgccacgttggggctgcctctgggaggatgacttcgaggctgccgccaaggaggccgcagccaaagaggctgccgctaagcttagtcgtctttttgataatgcaatgctccgtgcgcatcgcctccatcagctcgcttttgacacctaccaggagttcgaggaagcgtacattcctaaagaacagaaatactcgttcctccagaacccacagacctcgttatgtttcagtgaaagcattccaaccccatccaatcgggaagagacacagcaaaagtctaatttggagttgctccgcatttcgcttctccttattcagtcttggctcgaacctgttcagttcctccggtccgtctttgccaattccttagtctacggggcatcggatagcaatgtctatgaccttttgaaagacttggaagaacgcattcagactttgatggggcgtttggaagacggttcacctcgtacgggccagattttcaaacaaacttatagcaaatttgacacgaactcacataatgatgacgcattgctcaaaaactacgggctgctttactgctttcggaaggacatggataaggtggagacttttctgcggattgtacagtgtcggagcgtcgaagggagctgtggcttctaa

SEQ ID NO: 121 - protein, 6 aa, synthetic construct
HHHHHH

SEQ ID NO: 122 - DNA, 675 bp, synthetic construct
  source 1..675: mol_type = other DNA
catatgcagcgtctcattgaagatatttgtctgccgcgctggggctgcctctgggaagatgattttgaggctgccgccaaggaggccgcagccaaagaggctgccgctaagctgcgggacttgtttgaccgggcggttgtattgagtcactacattcataacttatcctccgaaatgttttctgagttcgataaacggtacacgcatggccgggggtttatcacaaaggccatcaactcatgccataccagtagcttggcgacaccggaggataaagagcaggctcaacagatgaaccagaaggacttcctgagccttattgtttccatcttgcggtcttggaatgaaccattatatcacttagtcaccgaagtgcgtgggatgcaagaagcgcctgaggcgattctcagtaaggccgtggaaatcgaagaacagaccaagcgtctgcttgaacggatggaacttattgtttcgcaagtccacccggagactaaggaaaacgaaatttatcctgtctggtcgggtcttccatctttacaaatggcagacgaagagtcgcggctttcggcgtattataacctcttgcattgcttacggcgggatagtcataagattgacaactacctcaaattacttaagtgccggattatccacaacaataactgttaactcgag

SEQ ID NO: 123 - protein, 184 aa, Homo sapiens
LRDLFDRAWLSHYIHNLSSEMFSEFDKRYTHGRGFITKAINSCHTSSLATPEDKEQAQQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLKCRIIHNNNC

SEQ ID NO: 124 - protein, 6 aa, synthetic construct
TIDEWL

(19) United States
(12) Patent Application Publication
(10) Pub. No.: US 2024/0409583 A1
(43) Pub. Date: Dec. 12, 2024

(54) ANTAGONIST FUSION POLYPEPTIDES

(71) Applicant: Kaida Biopharmaceuticals, Inc., Ft. Lauderdale, FL (US)

(72) Inventors: John Langenheim, Ft. Lauderdale, FL (US); Stephen Dunn, Pembroke Pines, FL (US)

(73) Assignee: Kaida Biopharmaceuticals, Inc., Ft. Lauderdale, FL (US)

(21) Appl. No.: 18/670,250

(22) Filed: May 21, 2024

Related U.S. Application Data
(60) Provisional application No. 63/468,209, filed on May 22, 2023.
(60) Provisional application No. 63/533,082, filed on Aug. 16, 2023.

(51) Int. Cl. C07K 7/08; A61K 38/00; A61P 35/00; C07K 14/575; C07K 14/61

(52) U.S. Cl. CPC C07K 7/08; A61P 35/00; C07K 14/57518; C07K 14/57554; C07K 14/61; A61K 38/00; C07K 2319/02

(57) ABSTRACT

The present disclosure provides antagonist fusion polypeptides (e.g., with extended half-life, improved antagonist activity) comprising (i) a serum albumin binding polypeptide; (ii) a polypeptide hormone analog capable of antagonizing its receptor activation; and (iii) a linker, as well as various related technologies including, methods of producing and using such fusion polypeptides.

### CROSS REFERENCE TO RELATED APPLICATIONS

This application claims the benefit of and priority to U.S. Provisional Patent Application No. 63/468,209, filed on May 22, 2023, and U.S. Provisional Patent Application 63/533,082, filed Aug. 16, 2023, the contents of each of which are incorporated herein by reference in their entirety.

### SEQUENCE LISTING

The instant application contains a Sequence Listing which has been submitted electronically in XML file format and is hereby incorporated by reference in its entirety. Said XML copy, created on Jun. 25, 2024, is named 135713-0104_SL.xml and is 150,224 bytes in size.

### BACKGROUND

The following description of the background of the present technology is provided simply as an aid in understanding the present technology and is not admitted to describe or constitute prior art to the present technology.

Prolactin (PRL), growth hormone (GH), and placental lactogen (PL) are structurally related hormones that each play important roles in, for example, growth control. These hormones can act as both classical endocrine modulators (e.g., hormones) via entry into the circulation and locally through juxtacrine, paracrine, and autocrine modes of action. Due, at least in part, to the structural similarities of PRL, GH, and PL, both GH and PL can bind to and activate each of the prolactin receptor (PRLR) and the growth hormone receptor (GHR), whereas PRL can bind to and activate only PRLR.

GHR and PRLR are both type I transmembrane glycoprotein cytokine receptor superfamily members and share substantially homology, especially in their extracellular domains, and in their interactions with kinases via their proximal intracellular domains. Ligand-mediated receptor homodimerization facilitates trans-phosphorylation of non-receptor tyrosine kinases belonging to the JAK kinase family and SRC kinase family (see, e.g., Kline, J. B., et al. Biol. Chem. 274:35461-35468, 1999) which can work in concert to activate intracellular signaling molecules that regulate cellular functions including, for example, survival, proliferation and differentiation which are important for the growth and development of target tissues.

Antagonism of PRLR and GHR signaling has been suggested as a means for treating certain diseases and/or disorders, such as cancer, including, for example, breast and gynecological cancers (see, e.g., Damiano and Wasserman. Clin. Cancer Res. 19 (7): 1644-1650, 2013; Divisova J et al., Breast Cancer Res Treat. 2006 August: 98 (3): 315-27). Several therapeutic strategies, including, for example, certain polypeptide hormone analogs, have been developed to inhibit the binding of ligands to PRLR and/or GHR. However, such polypeptide hormone analogs are rapidly cleared from the body; thus, their in vivo applications may be limited by their short circulatory half-life in vivo. Such short-acting biotherapeutics would require frequent dosing in humans by injection that can reduce applicability to the clinic, particularly for chronic conditions. Adverse side-effects of frequent dosing (e.g., daily administration) that could arise include, for example, injection site reaction and neutralizing antibodies as well as reduced patient compliance.

To advance technologies related to antagonism of PRLR and/or GHR signaling as a therapeutic strategy, it is important to develop long-acting polypeptide hormone analogs that antagonize their receptor, such as PRLR and/or GHR (e.g., with a longer half-life, with little or no partial agonism activity, with increased pharmacological effect), which could lead to, for example, reducing in dosing frequency, improved patient compliance, and/or improved overall treatment outcomes. Accordingly, there remains a need for such long-acting polypeptide hormone analogs and compositions.

### SUMMARY OF THE INVENTION

The present disclosure provides, among other things, fusion polypeptides comprising (i) a serum albumin binding polypeptide; (ii) a polypeptide hormone analog capable of antagonizing its receptor activation; and (iii) a linker adjoining (i) and (ii), wherein (i) is N-terminal to the linker and (ii) is C-terminal to the linker that can, for example, increase the half-life of such polypeptides (e.g., while maintaining or improving functional activity of both the serum albumin binding polypeptide and/or the polypeptide hormone analog) and/or increase pharmacological effect. The present disclosure also provides nucleic acids encoding such fusion polypeptides, compositions and kits comprising such fusion polypeptides and/or nucleic acids encoding the same, and methods of treating a disease and/or disorder.

In one aspect, the present disclosure provides a fusion polypeptide comprising: (i) a serum albumin binding polypeptide; (ii) a polypeptide hormone analog capable of antagonizing its receptor activation; and (iii) a linker adjoining (i) and (ii), wherein (i) is N-terminal to the linker and (ii) is C-terminal to the linker.

In some embodiments, the serum albumin binding polypeptide comprises an amino acid sequence of any one of SEQ ID NOs: 12-46.

In some embodiments, the polypeptide hormone analog is a prolactin (PRL) analog. In some embodiments, the PRL analog comprises an amino acid sequence of any one of SEQ ID NOs: 47-51.

In some embodiments, the polypeptide hormone analog is a growth hormone (GH) analog. In some embodiments, the GH analog comprises an amino acid sequence of SEQ ID NO: 54 or SEQ ID NO: 55.

In some embodiments, the polypeptide hormone analog is a placental lactogen (PL) analog. In some embodiments, the PL analog comprises an amino acid sequence of SEQ ID NO: 52 or SEQ ID NO: 53.

In some embodiments, the polypeptide hormone analog is N-terminally truncated relative to its parental polypeptide hormone.

In some embodiments, the linker is a rigid alpha-helical linker. In some embodiments, the rigid alpha-helical linker comprises an amino acid sequence of any one of SEQ ID NOs: 56-87.

In some embodiments, the fusion polypeptide further comprises a second linker, wherein the second linker is a flexible linker. In some embodiments, the flexible linker comprises an amino acid sequence of any one of SEQ ID NOs: 88-109.

In some embodiments, the serum albumin binding polypeptide has an affinity for serum albumin with a dissociation constant of less than about 200 nM.

In some embodiments, the polypeptide hormone analog has an affinity for its receptor with a dissociation constant of less than about 100 nM.

In some embodiments, the present disclosure provides a nucleic acid encoding a fusion polypeptide of the present disclosure. In some embodiments, an expression vector comprises the nucleic acid. In some embodiments, a host cell comprises the expression vector.

In one aspect, the present disclosure provides methods of producing a fusion polypeptide of the present disclosure comprising, contacting a cell with a nucleic acid encoding the fusion polypeptide, wherein contacting occurs under conditions sufficient to permit (i) uptake of the nucleic acid by the cell and (ii) translation of the fusion polypeptide.

In some embodiments, the present disclosure provides a composition comprising a fusion polypeptide of the present disclosure. In some embodiments, the composition is a pharmaceutical composition. In some embodiments, the pharmaceutical composition is an injectable pharmaceutical composition. In some embodiments, the pharmaceutical composition is a sustained release or long-lasting pharmaceutical composition.

In one aspect, the present disclosure provides a method for treating a disease and/or disorder in a subject comprising administering a therapeutically effective amount of a fusion polypeptide of the present disclosure. In some embodiments, the disease or disorder is a cancer. In some embodiments, the cancer is a chemotherapy-resistant cancer, a platinum-resistant chemotherapy, a chemotherapy-refractory cancer, or a platinum-refractory cancer. In some embodiments, the disease or disorder is a breast cancer or gynecological cancer. In some embodiments, the gynecological cancer is selected from the group consisting of: ovarian cancer, peritoneal cancer, cervical cancer, uterine cancers (e.g., endometrial cancer, uterine sarcoma), vaginal cancer, vulvar cancer, and fallopian tube cancer. In some embodiments, the gynecological cancer is ovarian cancer. In some embodiments, the ovarian cancer is a platinum-resistant ovarian cancer.

In some embodiments, the fusion polypeptide is administered peripherally.

In some embodiments, the fusion polypeptide is administered by subcutaneous injection.

In some embodiments, the fusion polypeptide is administered intravenously.

In some embodiments, the fusion polypeptide is administered in combination with a therapeutically effective amount of a second therapeutic agent. In some embodiments, the second therapeutic agent is a chemotherapy. In some embodiments, the chemotherapy is selected from the group consisting of: carboplatin, cisplatin, docetaxel, and paclitaxel.

In some embodiments, administering the fusion polypeptide and the second therapeutic agent in combination synergistically inhibits tumor growth compared to either the fusion polypeptide or the second therapeutic agent when administered as a monotherapy.

In some embodiments, the fusion polypeptide and the second therapeutic agent are administered at about the same time.

In some embodiments, the present disclosure provides a composition comprising a fusion polypeptide of the disclosure and a second therapeutic agent.

In one aspect, the present disclosure provides a method of maintaining or improving functional activity of a polypeptide hormone analog in a fusion polypeptide, comprising N-terminally truncating the polypeptide hormone analog relative to the parental polypeptide hormone.

In some embodiments, the present disclosure provides a kit comprising a fusion polypeptide of the present disclosure or a composition of the present disclosure.

In some embodiments, the present disclosure provides methods of characterizing a fusion polypeptide or composition of the present disclosure comprising assessing one or more of: (i) signaling activity of the fusion polypeptide; (ii) affinity of the fusion polypeptide to serum albumin; (iii) affinity of the fusion polypeptide to its receptor; (iv) purity of the fusion polypeptide; (v) yield of the fusion polypeptide; (vi) efficacy of treating a subject having a cancer; and (vii) half-life of the fusion polypeptide.

### BRIEF DESCRIPTION OF THE DRAWINGS

FIG. 1 shows an illustration of pET26-SA20-(EAAAK)3-G129R-hPRL (15-199) expression plasmid. The fusion gene encoding a serum albumin binding peptide (red), alpha-helical linker (yellow), and prolactin receptor antagonist analog (green) in which the N-terminus is truncated back to the alpha-helical region was inserted into the pET-26b(+) vector under the control of the T7lac promoter/operator. Features are indicated, including the E. coli replication origin (ori); the kanamcyin resistance (kanR); lac repressor (lacI).

FIG. 2 shows AlphaFold 2 prediction of SA20-(EAAAK)3-G129R-hPRL (15-199) folding structure. Rigid alpha helical linker (yellow) separates the 20 amino acid disulfide constrained serum albumin-binding peptide (red) from the pure PRLR antagonist (green). Figure discloses “(EAAAK)3” as SEQ ID NO: 58.

FIG. 3A-3B shows cloning and expression of SA20-(EAAAK)3-G129R-hPRL (15-199). (FIG. 3A) Agarose gel electrophoresis of isolated pET-26b(+) plasmids after restriction digestion with NdeI and XhoI for confirmation of SA20-(EAAAK)3-G129R-hPRL (15-199) insert (668 bp). (FIG. 3B) SDS-PAGE analysis of cell lysate from ten uninduced and 1 mM IPTG-induced BL21 (DE3) Star™ E. coli clones. Cells were grown for 4 h prior to induction with IPTG for an additional 3 h. Uninduced and induced cells were pelleted by centrifugation, resuspended in PBS and Laemmli sample buffer and heat-denatured. Lysate was loaded onto 12% polyacrylamide gels and separated by electrophoresis on a 12% polyacrylamide gel prior to staining with SYPRO Ruby Fluorescent Gel Stain.

FIG. 4A-4B shows the nucleotide and amino acid sequence verification of SA20-(EAAAK)3-G129R (hPRL 15-199) inserts in pET-26b(+). (FIG. 4A) Edited and trimmed Sanger sequencing chromatogram of pET26-SA20-(EAAAK)3-G129R-hPRL (15-199) clone sequenced with T7 promoter (shown) and T7 terminator primers (not shown). (FIG. 4B) DNA sequence was translated to amino acids and BLAST was used to confirm the identify the DNA sequence, which were confirmed to correctly encode the serum albumin binding peptide (SA20) and truncated human prolactin receptor antagonist (G129R (hPRL) 15-199)). Figure discloses SEQ ID NOS 122, 17, 17, 17, 123, 123, and 123, respectively, in order of appearance.

FIG. 5A-5B shows the isolation of SA20-(EAAAK)3-G129R-hPRL (15-199) from inclusion bodies and refolding of the fusion protein. (FIG. 5A) SDS-PAGE analysis of purified inclusion bodies containing SA20-(EAAAK)3-G129R-hPRL (15-199). Samples are as follows: lane 1) protein ladder, 2) wash 1 (w/triton) solubilized protein, 3) wash 2 (w/triton) solubilized protein, 4) wash 3 (w/triton) solubilized protein, 5) wash 4 (w/1 M urea) solubilized protein, 6) wash 5 (w/1 M urea) solubilized protein, 7) solubilized inclusion body. (FIG. 5B) Schematic diagram of protein refolding by urea gradient size exclusion chromatography (SEC). Inclusion bodies are solubilized in 8 M urea, reduced with DTT, and clarified by hi-speed centrifugation. The clarified supernatant protein content is quantitated prior to loading 50-100 mg of material onto the column in a 1-2 ml volume.

FIG. 6A-6B shows the analysis of rapidly refolded SA20-(EAAAK)3-G129R-hPRL (15-199) using urea gradient size-exclusion chromatography. (FIG. 6A) Chromatogram of SA20-(EAAAK)3-G129R-hPRL (15-199) refolding by urea gradient SEC. Solution A: 100 mM Tris (pH 8.0), 150 mM sodium chloride, 1 mM EDTA, 3 mM GSH, 0.3 mM GSSG. Solution B: solution A containing 8.0 M urea. Flow rate: 2.0 mL/min. Urea gradient: 2.0 M (25% B) to 8.0 M urea (100% B) in 80 mL. SA20-(EAAAK)3-G129R-hPRL (15-199) loading: 2 mL of SA20-(EAAAK)3-G129R-hPRL (15-199) in 8.0 M urea solution with a total protein concentration of 50 mg/mL. The green line is the conductivity curve, the red line is the UV 280 nm curve. The arrows show the refolded fusion protein and DTT. (FIG. 6B) Non-reducing SDS-PAGE analysis of eluted protein fractions using 12% polyacrylamide gel and stained with SYPRO Ruby Fluorescent Gel Stain.

FIG. 7 shows schematic diagram of protein purification following refolding by urea gradient size exclusion chromatography. Pooled fractions (tubes 2 thru 12) containing monomeric SA20-(EAAAK)3-G129R-hPRL (15-199) were dialyzed to remove residual urea and purified by ion exchange chromatography using Q FF column (1 mL).

FIG. 8A-8B shows purification of SA20-(EAAAK)3-G129R-hPRL (15-199) by ion exchange chromatography using a Q Sepharose FF column (Cytiva). (FIG. 8A) Protein elution was performed step-wise using 50 mM Tris-HCl, 1 mM EDTA (pH 8.0) containing 150 mM sodium chloride and monitored by absorbance 280 nM. The first absorbance peak primarily refolded SA20-(EAAAK)3-G129R-hPRL (15-199) and was analyzed by SDS-PAGE. (FIG. 8B) SDS-PAGE analysis of pooled fractions after filter sterilization. The gel shows the SA20-(EAAAK)3-G129R-hPRL (15-199) under non-reducing and fully reducing (DTT) conditions and under non-denaturing or fully denaturing conditions. The dialysis method leads to the formation of dimers and trimers that are not observed using the urea gradient SEC method.

FIG. 9 shows schematic diagram of protein refolding by dialysis and purification by ion exchange chromatography.

FIG. 10A-10B shows characterization of SA20-(EAAAK)3-G129R-hPRL (15-199) following purification by ion exchange chromatography using a Q Sepharose FF column (Cytiva). (FIG. 10A) Protein was loaded onto a Q Sepharose FF column, washed and elution was performed step-wise using 50 mM Tris-HCl, 1 mM EDTA (pH 8.0) containing 150 mM sodium chloride and monitored by absorbance 280 nM. The first absorbance peak primarily refolded SA20-(EAAAK)3-G129R-hPRL (15-199) and was analyzed by SDS-PAGE. (FIG. 10B) SDS-PAGE analysis of pooled fractions after filter sterilization. The gel shows the SA20-(EAAAK)3-G129R-hPRL (15-199) under non-reducing and fully reducing (DTT) conditions and under non-denaturing or fully denaturing conditions. The dialysis method leads to the formation of dimers and trimers that are not observed using the urea gradient SEC method.

FIG. 11A-11B shows schematic diagram of size exclusion chromatography and polishing step for purified SA20-(EAAAK)3-G129R-hPRL (15-199). (FIG. 11A) After ion exchange chromatography the SA20-(EAAAK)3-G129R-hPRL (15-199) was concentrated and applied to isolate monomeric fractions. (FIG. 11B) SDS-PAGE of purified SA20-(EAAAK)3-G129R-hPRL (15-199) fractions after SEC on Sephacryl S-100 column. The column was calibrated with Gel Filtration LMW Calibration Kit (Cytiva). Monomeric fractions were pooled and polished by ion exchange chromatography prior to filter sterilization and storage.

FIG. 12 shows immunoblot analysis of the phosphorylation status of STAT5A/B, AKT, and ERK1/2 in response to SA20-(EAAAK)3-G129R-hPRL (15-199). T-47D breast cancer cells were plated in RPMI medium 1640 containing 10% (v/v) charcoal/dextran-treated FBS overnight. The cells were depleted for 1 h in RPMI medium 1640 supplemented with 0.5% (v/v) CSS prior to treatment. The cells were treated for 30 min with 100 ng/ml hPRL in the absence or presence of increasing concentrations of SA20-(EAAAK)3-G129R-hPRL (15-199). Cell lysates were harvested, and 40 μg of lysate from each treatment was separated by reducing SDS-PAGE using 4-15% polyacrylamide gels and transferred to nitrocellulose membranes. The membranes were probed with antibodies recognizing phospho-Akt1/2/3, phospho-ERK1/2, and phospho-STAT5A/B. Equal loading was confirmed by probing for GAPDH. SA20-(EAAAK)3-G129R-hPRL (15-199) was able to competitively inhibit the effects of PRL in dose-dependent manner.

FIG. 13 shows the semi-log plot of mean late plasma concentrations versus time profiles of G129R-hPRL (1-199) and fusion polypeptide, SA20-(EAAAK)3-G129R-hPRL (15-199), after i.p. administration of a single molar equivalent dose to FVB/N mice (n=6). The plasma concentrations were determined by ELISA and the β-phase was used to determine the elimination half-life of the polypeptides.

### DETAILED DESCRIPTION

The prolactin receptor (PRLR) and growth hormone receptor (GHR) are a single-transmembrane domain type 1 cytokine receptors located on the plasma membrane of cells. The PRLR can be activated by three small polypeptide hormones (191-199 amino acid residues in length): prolactin (PRL), growth hormone (GH) and placental lactogen (PL), while GHR can be activated by either of GH and PL. Ligand-mediated receptor homodimerization facilitates trans-phosphorylation of non-receptor tyrosine kinases belonging to the JAK kinase family and SRC kinase family (Kline, J. B., et al. Biol. Chem. 274:35461-35468, 1999) which can work in concert to activate intracellular signaling molecules that regulate cellular functions including, for example, survival, proliferation and differentiation, which are important for the growth and development of target tissues.

Antagonism of PRLR and GHR signaling has been suggested as a means for treating cancer, including, for example breast and gynecological cancers (see, e.g., Damiano and Wasserman. Clin. Cancer Res. 19 (7): 1644-1650, 2013; Divisova J et al., Breast Cancer Res Treat. 2006 August; 98 (3): 315-27). However, polypeptide hormones such as PRL, GH, and PL, as well as antagonist analogs, are cleared by the kidneys within a few minutes of entering the patient's blood. Prolactin and growth hormone have a bloodstream half-life of only 20-50 minutes when infused. Several therapeutic strategies, including, for example, certain polypeptide hormone analogs, have been developed to inhibit the binding of ligands to PRLR and/or GHR and subsequent downstream signaling. Though, such polypeptide hormone analogs are also rapidly cleared from the body. In addition, many PRL, GH, and PL antagonist analogs still maintain some degree of partial agonism activity.

Such short serum half-lives in vivo necessitate the administration of such therapeutics to date at high frequencies (e.g., daily) and/or higher doses to maintain the serum levels necessary for therapeutic effects. This frequent systemic administration of drugs is associated with considerable negative side effects. For example, frequent (e.g. daily), subcutaneous injections represent a considerable discomfort to the subject, can pose a high risk of administration related infections, and may require hospitalization and/or frequent visits to the hospital. Moreover, in long term treatments, daily injections (e.g., subcutaneous injections) can also lead to considerable side effects, such as tissue scarring and vascular pathologies caused by the repeated puncturing of vessels. All these factors lead to decreased patient compliance and/or increased costs for the health system.

To advance technologies related to antagonism of PRLR and/or GHR signaling as a therapeutic strategy, it is important to develop long-acting (e.g., long half-life) polypeptide hormone analogs that antagonize their receptor (e.g., PRLR and/or GHR), which could lead to, for example, reducing in dosing frequency, improved compliance, and/or improved overall treatment outcomes. Accordingly, there remains a need for such long-acting polypeptide hormone analogs and related compositions.

The present disclosure provides, among other things, fusion polypeptides comprising (i) a serum albumin binding polypeptide; (ii) a polypeptide hormone analog capable of antagonizing its receptor activation; and (iii) a linker adjoining (i) and (ii), wherein (i) is N-terminal to the linker and (ii) is C-terminal to the linker that can, for example, increase the half-life of such polypeptides (e.g., while maintaining or improving functional activity of the serum albumin binding polypeptide and/or the polypeptide hormone analog as an antagonist) and/or increase pharmacological effect. The present disclosure also provides, for example, polynucleotides encoding such fusion polypeptides, compositions and kits comprising such fusion polypeptides and/or nucleic acids encoding the same, and methods of treating a disease and/or disorder.

It is to be appreciated that certain aspects, modes, embodiments, variations and features of the present methods are described below in various levels of detail to provide a substantial understanding of the present technology.

The present disclosure is not to be limited in terms of the particular embodiments described in this application, which are intended as single illustrations of individual aspects of the disclosure. All the various embodiments of the present disclosure will not be described herein. Many modifications and variations of the disclosure can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled.

It is to be understood that the present disclosure is not limited to particular uses, methods, reagents, compounds, compositions or biological systems, which can, of course, vary. It is also to be understood that the terminology used herein is for the purpose of describing particular embodiments only and is not intended to be limiting.

In practicing the present methods, many conventional techniques in molecular biology, protein biochemistry, cell biology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

### Definitions

Unless defined otherwise, all technical and scientific terms used herein have the meaning commonly understood by a person skilled in the art to which this disclosure belongs. The following references provide one of skill with a general definition of many of the terms used in the present disclosure. Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise. The terminology used herein is for the purpose of describing particular embodiments only and is not intended to be limiting of the disclosure.

As used herein, the term “administration” of an agent (e.g., fusion polypeptide as described herein) or composition to a subject includes any route of introducing or delivering the agent or composition to a subject to perform its intended function. Administration can be carried out by any suitable route, including, but not limited to, intravenously, intramuscularly, intraperitoneally, subcutaneously, and other suitable routes as described herein. Administration can include self-administration and administration by another.

As used herein, the term “affinity”, refers to a measure of the tightness with which two or more binding partners associate with one another. Those skilled in the art are aware of a variety of assays that can be used to assess affinity, and will furthermore be aware of appropriate controls for such assays. For example, affinity can assessed in a quantitative assay, over a plurality of concentrations (e.g., of one binding partner at a time), in the presence of one or more potential competitor entities (e.g., that might be present in a relevant—e.g., physiological-setting), relative to a reference (e.g., that has a known affinity above a particular threshold [a “positive control” reference] or that has a known affinity below a particular threshold [a “negative control” reference” ]). Typically, when affinity is assessed relative to a reference, it is assessed under comparable conditions.

As used herein, the term “agent,” refers to an entity (e.g., for example, a lipid, metal, nucleic acid, polypeptide, polysaccharide, small molecule, etc., or complex, combination, mixture or system ([e.g., cell, tissue, organism] thereof), or phenomenon (e.g., heat, electric current or field, magnetic force or field, etc.). In appropriate circumstances, as will be clear from context to those skilled in the art, the term may be utilized to refer to an entity that is or comprises a cell or organism, or a fraction, extract, or component thereof (e.g., nucleic acids). Alternatively or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. For example, an agent may be utilized in isolated or pure form; an agent may be utilized in crude form.

As used herein, the term “approximately” or “about” means plus or minus 10% as well as the specified number. For example, “about 10” should be understood as both “10” and “9-11”.

As used herein, the term an “amino acid substitution” or “substituted” (when such term is referred to a substituted amino acid) refers to the replacement of at least one existing amino acid residue in a predetermined amino acid sequence with a different amino acid residue in a predetermined amino acid sequence with a different amino acid residue. The term “amino acid insertion” refers to the incorporation of at least one additional amino acid into a predetermined amino acid sequence. While the insertion will usually consist of the insertion of one or two amino acid residues, larger “peptide insertions,” can also be made. The replaced or inserted amino acid residue(s) may be naturally occurring or non-naturally occurring (modified). The term “amino acid deletion” refers to the removal of at least one amino acid residue from a predetermined amino acid sequence.

As used herein, the term “analog” refers to a substance that shares one or more particular structural features, elements, components, or moieties with a reference or “parent” substance. Typically, an “analog” shows significant structural similarity with the reference substance, for example sharing a core or consensus structure, but also differs in certain discrete ways. An analog can be a substance that can be generated from the reference substance, e.g., by chemical and/or genetic manipulation of the reference substance (e.g., substitution, deletion, and/or insertion of one or more amino acid residues of a polypeptide hormone relative to a parent polypeptide hormone, thereby generating a polypeptide hormone analog). An analog can be a substance that can be generated through performance of a synthetic process substantially similar to (e.g., sharing a plurality of steps with) one that generates the reference substance. An analog is or can be generated through performance of a synthetic process different from that used to generate the reference substance.

As used herein, the term “antagonist” refers to a receptor antagonist (e.g., a prolactin receptor and/or growth hormone receptor antagonist), which is a type of receptor ligand or agent (e.g., a fusion polypeptide; a polypeptide hormone analog capable of antagonizing its receptor activation as described herein) that blocks or dampens agonist-mediated responses rather than provoking a biological response itself upon binding to a receptor. In pharmacology, antagonists have affinity, but no efficacy for their cognate receptors, and binding will disrupt the interaction and inhibit the function of an agonist or inverse agonist at receptors. Antagonists mediate their effects by binding to the active (orthosteric) site or to allosteric sites on receptors, or they may interact at unique binding sites not normally involved in the biological regulation of the receptor's activity. Antagonist activity may be reversible or irreversible depending on the longevity of the antagonist-receptor complex, which, in turn, depends on the nature of antagonist-receptor binding. The majority of antagonists achieve their potency by competing with endogenous ligands or substrates at structurally defined binding sites on receptors.

As used herein, the term “binding”, refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts-including where interacting entities or moieties are studied in isolation or in the context of more complex systems (e.g., while covalently, electrostatically, or otherwise associated with a carrier entity and/or in a biological system or cell). Binding between two entities may be considered “specific” if, under the conditions assessed, the relevant entities are more likely to associate with one another than with other available binding partners.

As used herein, the term “combination therapy” refers to those situations in which a subject is simultaneously exposed to two or more therapeutic regimens (e.g., a fusion polypeptide as described herein and a second therapeutic agent). For example, the two or more regimens may be administered simultaneously, sequentially (e.g., all “doses” of a first regimen are administered prior to administration of any doses of a second regimen), or in overlapping dosing regimens. “Administration” of combination therapy may involve administration of one or more agent(s) or modality (ies) to a subject receiving the other agent(s) or modality (ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

As used herein, a “conservative amino acid substitution” is one in which the amino acid residue is replaced with an amino acid residue having a similar side chain. Families of amino acid residues having similar side chains have been defined in the art, including: (1) hydrophobic side chains: norleucine, Met, Ala, Val, Leu; (2) neutral hydrophilic side chains: Cys, Ser, Thr, Asn, Gln; (3) acidic side chains: Asp, Glu; (4) basic side chains: His, Lys, Arg; (5) side chains that influence chain orientation: Gly, Pro; and (6) aromatic side chains: Trp, Tyr, Phe. For example, a non-conservative amino acid substitution is a substitution of an amino acid residue with a substantially different side chain (i.e., an amino acid residue that is a member of a different family). In some embodiments, a conservative amino acid substitution is made by considering the hydropathic index of the amino acid residue. Each amino acid is assigned a hydropathic index on the basis of its hydrophobicity and charge characteristics. They are: lie (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glu (−3.5); Gin (−3.5); Asp (−3.5); Asn (−3.5); Lys (−3.9); and Arg (−4.5). The importance of the hydropathic amino acid index in conferring interactive function on a polypeptide is understood in the art (see, e.g., Kyte et al (1982) J Mol Biol 157:105-131). In some embodiments, a conservative amino acid substitution is made by replacing one amino acid residue with another amino acid residue having a the same or similar (e.g., within about +2, +1.5, +1, +0.5, −0.5, −1, −1.5, or −2) hydropathic index. In some embodiments, a conservative amino acid substitution is made by considering the hydrophilicity of the amino acid residue. The following hydrophilicity values have been assigned: Arg (+3.0); Lys (+3.0+1); Asp (+3.0+1); Glut (+0.2); Gly (0); Thr (−0.4); Pro (−0.5+1); Ala (−0.5); His (−0.5); Cys (−1.0); Met (−1.3); Val (−1.5); Leu (−1.8); lie (−1.8); Tyr (−2.3); Phe (−2.5); and Trp (−3.4). In some embodiments, a conservative amino acid substitution is made by replacing one amino acid residue with another amino acid residue having a the same or similar (e.g., within about +2, +1.5, +1, +0.5, −0.5, −1, −1.5, or −2) hydrophilicity. Exemplary amino acid substitutions are set forth in Table 1:

TABLE 1
Conservative amino acid substitutions

| Original | | Preferred |
|---|---|---|
| Residue | Exemplary Substitution | Substitution |
| Ala | Val, Leu, Ile | Val |
| Arg | Lys, Gln, Asn | Lys |
| Asn | Gln | Gln |
| Asp | Gln | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, Phe, Nle | Leu |
| Leu | Nle, Ile, Val, Met, Ala, Phe | Ile |
| Lys | Arg, Dbu, Gln, Asn | Arg |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, Nle | Leu |

Nle = norleucine
Dbu = 2,4-diaminobutyric acid

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.”

As used herein, the term “expression” of a nucleic acid sequence refers to the generation of any gene product from the nucleic acid sequence. A gene product can be a transcript. A gene product can be a polypeptide. Expression of a nucleic acid sequence can involve one or more of the following: (1) production of an RNA template from a DNA sequence (e.g., by transcription); (2) processing of an RNA transcript (e.g., by splicing, editing, etc.); (3) translation of an RNA into a polypeptide; and/or (4) post-translational modification of a polypeptide.

As used herein, the term “fragment” refers to a material or entity as described herein that has a structure that includes a discrete portion of the whole, but lacks one or more moieties found in the whole. For example, a fragment can consist of such a discrete portion. A fragment can consist of or comprise a characteristic structural element or moiety found in the whole. For example, a polypeptide fragment can comprise or consist of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) as found in the whole polypeptide. A polypeptide fragment can comprise or consist of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the whole polypeptide. The whole material or entity may be referred to as the “parent” of the fragment.

As used herein, the term “functional”, refers to a form or fragment of an entity (e.g., biomolecular cargo) that exhibits a particular property and/or activity.

As used herein, the term “host cell” refers to a cell into which exogenous DNA (recombinant or otherwise) has been introduced. Persons of skill upon reading this disclosure will understand that such terms refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. Host cells can include any prokaryotic and eukaryotic cells that are suitable for expressing an exogenous DNA (e.g., a recombinant nucleic acid sequence). Exemplary cells include those of prokaryotes and eukaryotes (single-cell or multiple-cell), bacterial cells (e.g., strains of E. coli, Bacillus spp., Streptomyces spp., etc.), mycobacteria cells, fungal cells, yeast cells (e.g., S. cerevisiae, S. pombe, P. pastoris, P. methanolica, etc.), plant cells, insect cells (e.g., SF-9, SF-21, baculovirus-infected insect cells, etc.), non-human animal cells (e.g., Chinese Hamster Ovary cells), human cells (e.g., Human Embryonic Kidney (HEK) cells), or cell fusions such as, for example, hybridomas or quadromas.

As used herein, the term “effective amount” or “therapeutically effective amount” refers to a quantity of an agent or composition sufficient to achieve a beneficial or desired clinical result upon treatment. In the context of therapeutic applications, the amount of a therapeutic agent administered to the subject can depend on, for example, the type and/or severity of the disease and/or disorder and/or on the characteristics of the individual, such as general health, age, sex, body weight, effective concentration of the agent (e.g., fusion polypeptide) administered, and/or tolerance to drugs. It can also depend on the degree, severity, and/or type of disease. The skilled artisan will be able to determine appropriate dosages depending on these and other factors. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art.

As used herein, the terms “improved”, “increased”, or “reduced”, or grammatically comparable comparative terms, indicate values that are relative to a comparable reference measurement. For example, in some embodiments, an assessed value achieved with an agent of interest may be “improved” relative to that obtained with a comparable reference agent. Alternatively or additionally, in some embodiments, an assessed value achieved in a subject or system of interest may be “improved” relative to that obtained in the same subject or system under different conditions (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc.). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

As used herein, the term “isolated”, “purified”, or “biologically pure” refers to material that is free to varying degrees from components which normally accompany it as found in its native and/or original state (e.g., undesired sample components). “Isolate” denotes a degree of separation from original source or surroundings. “Purify” denotes a degree of separation that is higher than isolation. For example, a “purified” or “biologically pure” polypeptide is sufficiently free of other materials such that any impurities do not materially affect the biological properties of the polypeptide or cause other adverse consequences. That is, a fusion polypeptide of the presently disclosed subject matter is purified if it is substantially free of undesired sample components, such as cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis. The term “purified” can denote that a nucleic acid or polypeptide gives rise to essentially one band in an electrophoretic gel. For a polypeptide that can be subjected to modifications, for example, phosphorylation or lipidation, different modifications may give rise to different isolated polypeptides, which can be separately purified.

As used herein, the term “operably linked”, refers to a juxtaposition wherein the components described are in a relationship permitting them to function in their intended manner. A control element “operably linked” to a functional element is associated in such a way that expression and/or activity of the functional element is achieved under conditions compatible with the control element. In some embodiments, “operably linked” control elements are contiguous (e.g., covalently linked) with the coding elements of interest; in some embodiments, control elements act in trans to or otherwise at a from the functional element of interest.

As used herein, the term “percent (%) amino acid sequence identity” or “percent sequence identity” with respect to a reference polypeptide or nucleic acid sequence is defined as the percentage of amino acid residues or nucleotides in a candidate sequence that are identical with the amino acid residues or nucleotides in the reference polypeptide or nucleic acid sequence, after aligning the sequences and introducing gaps, if necessary, to achieve the maximum percent sequence identity. Alignment for purposes of determining percent amino acid sequence identity or percent sequence identity can be achieved in various ways that are known in the art, for example, using publicly available computer software such as BFASTp, BFAST-2, AFIGN (e.g., AFIGN-2) or Megalign (DNASTAR) software. To obtain gapped alignments for comparison purposes, Gapped BEAST can be utilized as described in Altschul et al, (1997) Nucleic Acids Res. 25 (17): 3389-3402. In addition, the percent identity between two amino acid sequences or nucleic acid sequences can be determined using the Needleman and Wunsch (J. Mol. Biol. (48): 444-453 (1970)) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6 or EMBOSS Needle.

As used herein, the term “pharmaceutical composition” refers to an active agent (e.g., a fusion polypeptide as described herein), formulated together with one or more pharmaceutically acceptable carriers. An active agent can be present in unit dose amount appropriate for administration in a therapeutic regimen that shows a statistically significant probability of achieving a predetermined therapeutic effect when administered to a relevant population. Pharmaceutical compositions may be specially formulated for administration in, for example, solid or liquid form, including those adapted for a particular route of administration.

As used herein, the term “pharmacological effect” refers to a change which is a result or consequence of an agent (e.g., a fusion polypeptide described herein). Such a change may be a biological change on, for example, cells, organs, and/or systems. A “pharmacological effect” can be the result of a specific biochemical interaction of an agent and its target (e.g., a fusion polypeptide and its receptor).

As used herein the term, “platinum-based therapy” or “platinum-based cancer therapy” or “platinum-based treatment” refers to treatment using anti-cancer agents that comprise the metal, platinum. Examples of platinum-based therapies include, cisplatin, carboplatin, and oxaliplatin.

The terms “polypeptide”, “peptide”, and “protein” are used interchangeably herein to refer to a polymer of amino acid residues. The terms apply to naturally occurring amino acid polymers as well as amino acid polymers in which one or more amino acid residues are a non-naturally occurring amino acid, e.g., an amino acid analog. The terms encompass amino acid chains of any length, including full-length proteins or fragments thereof, wherein the amino acid residues are linked by covalent peptide bonds.

As used herein, the term “Progression-Free Interval” or “PFI”, refers to the amount of time that has elapsed between the completion of a platinum- or non-platinum-based therapy and the detection of relapse.

As used herein, the term “reduce” or “decrease” means to alter negatively by at least about 5% including, but not limited to, alter negatively by about 5%, by about 10%, by about 25%, by about 30%, by about 50%, by about 75%, or by about 100%.

As used herein, a “reference” entity, system, amount, set of conditions, etc., is one against which a test entity, system, amount, set of conditions, etc. is compared as described herein. For example, in some embodiments, a “reference” polypeptide is a control polypeptide, e.g., a polypeptide hormone analog not part of a fusion polypeptide described herein, a parental polypeptide hormone. In some embodiments, a reference or control is tested and/or determined simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

As used herein, the term “resistant” or “resistance” in the context of a cancer, refers to cancer with a reduction in the effectiveness of a treatment to the cancer, e.g., a particular agent in treating cancer. A subject with a Progression Free Interval of less than six months is considered to have a resistant cancer (e.g., chemotherapy-resistant cancer, platinum-resistant cancer).

As used herein, the term “refractory” refers to a resistant cancer wherein the cancer progresses (e.g., spreads, grows, metastasizes) while the subject with cancer is on a platinum- or non-platinum-based therapy (a platinum-refractory cancer, a chemotherapy-refractory cancer, respectively).

As used herein, the terms “subject”, “individual”, or “patient” are used interchangeably and refer to an individual organism, a vertebrate, or a mammal and may include humans, non-human primates, rodents, and the like (e.g., which is to be the recipient of a particular treatment). In certain embodiments, the individual, patient or subject is a human.

As used herein, “sustained release” refers to a pharmaceutical composition or compound that conforms to a release rate over a specific period of time. The period of time may include, but is not limited to, hours, days, weeks, months and years.

As used herein, the term “treatment” (also “treat” or “treating”) refers to any administration of a substance (e.g., fusion polypeptides, nucleic acids encoding fusion polypeptides of the present disclosure) that partially or completely alleviates, ameliorates, relieves, inhibits, delays onset of, reduces severity of, and/or reduces incidence of one or more symptoms, features, and/or causes of a particular disease, disorder, and/or condition (e.g., cancer). Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

As used herein, in the context of molecules (e.g., nucleic acids, polypeptides), the term “variant” refers to a molecule that shows significant structural identity with a reference molecule, but differs structurally from the reference molecule, e.g., in the presence or absence or in the level of one or more chemical moieties as compared to the reference entity. A variant can also differ functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements, but differs in at least one aspect from the reference molecule. For example, a polypeptide may have a characteristic sequence element comprised of a plurality of amino acids having designated positions relative to one another in linear or three-dimensional space and/or contributing to a particular structural motif and/or biological function; a nucleic acid may have a characteristic sequence element comprised of a plurality of nucleotide residues having designated positions relative to on another in linear or three-dimensional space. A variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. A reference polypeptide or nucleic acid can have one or more biological activities. A variant polypeptide or nucleic acid can share one or more of the biological activities of the reference polypeptide or nucleic acid. A variant polypeptide or nucleic acid can lack one or more of the biological activities of the reference polypeptide or nucleic acid. A variant polypeptide or nucleic acid can show a reduced level of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide or nucleic acid comprises about 10, about 9, about 8, about 7, about 6, about 5, about 4, about 3, about 2, or about 1 substituted residues as compared to a reference. Often, a variant polypeptide or nucleic acid comprises a very small number (e.g., fewer than about 5, about 4, about 3, about 2, or about 1) number of substituted, inserted, or deleted, functional residues (i.e., residues that participate in a particular biological activity) relative to the reference. In some embodiments, a variant polypeptide or nucleic acid comprises not more than about 5, about 4, about 3, about 2, or about 1 addition or deletion, and, in some embodiments, comprises no additions or deletions, as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, a reference polypeptide or nucleic acid is one found in nature. In some embodiments, a reference polypeptide or nucleic acid is a human polypeptide or nucleic acid.

As used herein, the term “vector” has the same meaning as commonly understood by one of ordinary skill in the art, and refers to a nucleic acid molecule capable of transporting another nucleic acid molecule to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors used in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

### Fusion Polypeptides

Fusion polypeptides of the present disclosure can comprise (i) a serum albumin binding polypeptide; (ii) a polypeptide hormone analog capable of antagonizing its receptor activation; and (iii) a linker adjoining (i) and (ii), wherein (i) is N-terminal to the linker and (ii) is C-terminal to the linker. Any serum albumin binding polypeptides, polypeptide hormone analogs, and linkers or variants thereof described herein may be utilized in any combination as part of a fusion polypeptide.

In some embodiments, fusion polypeptides of the present disclosure have a prolonged in vivo half-life relative to a reference (e.g., a polypeptide hormone analog not part of a fusion polypeptide as described herein). In some such embodiments, fusion polypeptides of the present disclosure have an in vivo half-life at least about 2-fold, 3-fold, 4-fold, 5-fold, 6-fold, 7-fold, 8-fold, 9-fold, 10-fold, 15-fold, 20-fold, 25-fold, or 50-fold higher relative to a reference (e.g., a reference which has a half-life of 20-50 minutes). In some such embodiments, fusion polypeptides of the present disclosure have an in vivo half-life at least about 2-fold higher relative to a reference. In some such embodiments, fusion polypeptides of the present disclosure have an in vivo half-life at least about 3-fold higher relative to a reference. In some such embodiments, fusion polypeptides of the present disclosure have an in vivo half-life at least about 4-fold higher relative to a reference. In some such embodiments, fusion polypeptides of the present disclosure have an in vivo half-life at least about 5-fold higher relative to a reference. In some such embodiments, fusion polypeptides of the present disclosure have an in vivo half-life at least about 6-fold higher relative to a reference. In some such embodiments, fusion polypeptides of the present disclosure have an in vivo half-life at least about 7-fold higher relative to a reference. In some such embodiments, fusion polypeptides of the present disclosure have an in vivo half-life at least about 8-fold higher relative to a reference. In some such embodiments, fusion polypeptides of the present disclosure have an in vivo half-life at least about 9-fold higher relative to a reference. In some such embodiments, fusion polypeptides of the present disclosure have an in vivo half-life at least about 10-fold higher relative to a reference. In some such embodiments, fusion polypeptides of the present disclosure have an in vivo half-life at least about 15-fold higher relative to a reference. In some such embodiments, fusion polypeptides of the present disclosure have an in vivo half-life at least about 20-fold higher relative to a reference. In some such embodiments, fusion polypeptides of the present disclosure have an in vivo half-life at least about 25-fold higher relative to a reference. In some such embodiments, fusion polypeptides of the present disclosure have an in vivo half-life at least about 50-fold higher relative to a reference.

In some embodiments, fusion polypeptides of the present disclosure that associate with a serum albumin (e.g., human serum albumin) exhibit a serum half-life of at least about 50%, at least about 60%, at least about 70%, at least about 80%, or at least about 90% of the natural half-life of the serum albumin (e.g., human serum albumin) in a subject. In some embodiments, fusion polypeptides of the present disclosure that associate with serum albumin exhibit a serum half-life of at least about 50% of the natural half-life of the serum albumin in a subject. In some embodiments, fusion polypeptides of the present disclosure that associate with serum albumin exhibit a serum half-life of at least about 60% of the natural half-life of the serum albumin in a subject. In some embodiments, fusion polypeptides of the present disclosure that associate with serum albumin exhibit a serum half-life of at least about 70% of the natural half-life of the serum albumin in a subject. In some embodiments, fusion polypeptides of the present disclosure that associate with serum albumin exhibit a serum half-life of at least about 80% of the natural half-life of the serum albumin in a subject. In some embodiments, fusion polypeptides of the present disclosure that associate with serum albumin exhibit a serum half-life of at least about 90% of the natural half-life of the serum albumin in a subject.

In many instances, it is understood that incorporation of a polypeptide (e.g., a polypeptide hormone analog) as a part of a fusion polypeptide can decrease the activity (e.g., antagonist activity) of the polypeptide relative to the polypeptide when not a part of a fusion polypeptide (e.g., a fusion polypeptide described herein). Without wishing to be bound by any one theory, the present disclosure understands that a decrease in antagonistic activity of polypeptide hormone analogs as part of a fusion polypeptide can be a result of the N-terminal and C-terminal proximal tails of the four-helix bundle hormone analogs. The C-terminal proximal tail is part of the high affinity receptor binding site 1 of the polypeptide hormone analog and is important for competitive antagonism, C-terminal fusions reduce the high affinity receptor binding site substantially which is detrimental to antagonist activity. The N-terminal proximal tail is part of the low affinity receptor binding site 2, which is already disrupted in polypeptide hormone analogs by mutation in another region of receptor binding site 2, making the N-terminal tail dispensable for antagonism. Without wishing to be bound by any one theory, it is believed that fusions to the N-terminus of the polypeptide hormone analogs cause the N-terminal tails to fold back on the central alpha-helix bundle, which sterically hinders receptor binding site 1 of the hormone analogs, reducing antagonist activity to less of an extent than C-terminal fusions.

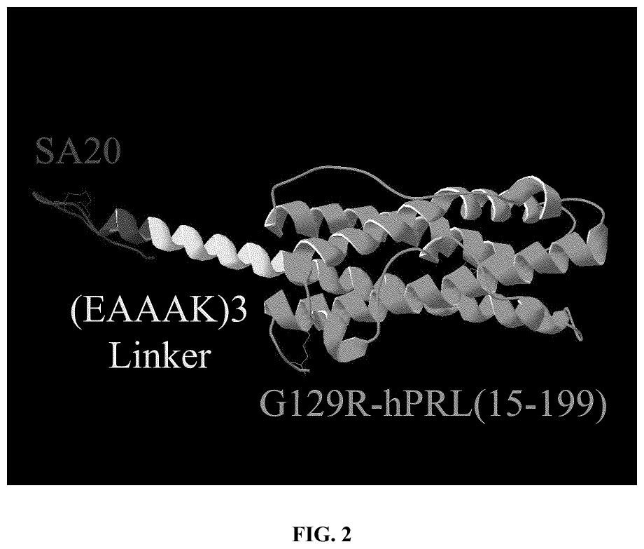

Thus, the present disclosure provides, among other things, fusion polypeptides comprising polypeptide hormone analogs that maintain or improve antagonistic activity of the polypeptide hormone analog (e.g., relative to a polypeptide hormone analog that is not part of a fusion polypeptide as described herein). Without wishing to be bound by any one theory, the present disclosure understands that maintenance or improvement of antagonistic activity of the polypeptide hormone analog as part of a fusion polypeptide can be achieved by removing (or deleting) the unstructured N-terminus of hormone analogs and replacing it with a linker (e.g., a rigid linker) that extends the fourth alpha-helix away from the polypeptide hormone analog to prevent or reduce steric hinderance (FIG. 2). Accordingly, and without wishing to be bound by any one theory, it is understood that such a configuration of the fusion polypeptides described herein can facilitate both of (i) binding to a serum albumin (e.g., to improve in serum half-life); and/or (ii) maintenance or improvement of polypeptide hormone analog antagonistic activity.

Examples of amino acid sequences of fusion polypeptides of the disclosure are shown in Table 2. In some embodiments, a fusion polypeptide of the present disclosure comprises an amino acid sequence at least 60%, at least 65%, at least 70%, at least 75%, at least 80%, at least 85%, at least 90%, at least 95%, at least 96%, at least 97%, at least 98%, or at least 99% identical to any one of SEQ ID NOs: 1-11. In some embodiments, a fusion polypeptide of the present disclosure comprises the amino acid sequence of any one of SEQ ID NOs: 1-11 or a variant thereof.

TABLE 2
Examples of amino acid sequences of fusion
polypeptides.

| SEQ | Fusion | |
|---|---|---|
| ID | poly- | |
| NO: | peptide | Amino acid sequence |
| 1 | SA20- | MQRLIEDICLPRWGCLWEDDFGGGGSGGGGS |
| | (GGGGS)₃- | GGGGSLRDLFDRAVVLSHYIHNLSSEMFSEF |
| | G129R- | DKRYTHGRGFITKAINSCHTSSLATPEDKEQ |
| | hPRL | AQQMNQKDFLSLIVSILRSWNEPLYHLVTEV |
| | (15-199) | RGMQEAPEAILSKAVEIEEQTKRLLERMELI |
| | | VSQVHPETKENEIYPVWSGLPSLQMADEESR |
| | | LSAYYNLLHCLRRDSHKIDNYLKLLKCRIIH |
| | | NNNC* |
| 2 | SA20- | MQRLIEDICLPRWGCLWEDDFGGGGSGGGGS |
| | (GGGGS)₄- | GGGGSGGGGSLRDLFDRAVVLSHYIHNLSSE |
| | G129R- | MFSEFDKRYTHGRGFITKAINSCHTSSLATP |
| | hPRL | EDKEQAQQMNQKDFLSLIVSILRSWNEPLYH |
| | (15-199) | LVTEVRGMQEAPEAILSKAVEIEEQTKRLLE |
| | | RMELIVSQVHPETKENEIYPVWSGLPSLQMA |
| | | DEESRLSAYYNLLHCLRRDSHKIDNYLKLLK |
| | | CRIIHNNNC* |
| 3 | SA20- | MQRLIEDICLPRWGCLWEDDFEAAAKEAAAK |
| | (EAAAK)₃- | EAAAKLRDLFDRAVVLSHYIHNLSSEMFSEF |
| | G129R- | DKRYTHGRGFITKAINSCHTSSLATPEDKEQ |
| | hPRL | AQQMNQKDFLSLIVSILRSWNEPLYHLVTEV |
| | (15-199) | RGMQEAPEAILSKAVEIEEQTKRLLERMELI |
| | | VSQVHPETKENEIYPVWSGLPSLQMADEESR |
| | | LSAYYNLLHCLRRDSHKIDNYLKLLKCRIIH |
| | | NNNC* |
| 4 | SA20- | MQRLIEDICLPRWGCLWEDDFAEAAAKEAAA |
| | A(EAAAK)₂A- | KALRDLFDRAVVLSHYIHNLSSEMFSEFDKR |
| | G129R- | YTHGRGFITKAINSCHTSSLATPEDKEQAQQ |
| | hPRL | MNQKDFLSLIVSILRSWNEPLYHLVTEVRGM |
| | (15-199) | QEAPEAILSKAVEIEEQTKRLLERMELIVSQ |
| | | VHPETKENEIYPVWSGLPSLQMADEESRLSA |
| | | YYNLLHCLRRDSHKIDNYLKLLKCRIIHNNN |
| | | C* |
| 5 | SA20- | MQRLIEDICLPRWGCLWEDDFAEAAAKEAAA |
| | A(EAAAK)₃A- | KEAAAKALRDLFDRAVVLSHYIHNLSSEMFS |
| | G129R- | EFDKRYTHGRGFITKAINSCHTSSLATPEDK |
| | hPRL | EQAQQMNQKDFLSLIVSILRSWNEPLYHLVT |
| | (15-199) | EVRGMQEAPEAILSKAVEIEEQTKRLLERME |
| | | LIVSQVHPETKENEIYPVWSGLPSLQMADEE |
| | | SRLSAYYNLLHCLRRDSHKIDNYLKLLKCRI |
| | | IHNNNC* |
| 6 | SA20- | MQRLIEDICLPRWGCLWEDDFAEAAAKEAAA |
| | A(EAAAK)₄A- | KEAAAKEAAAKALRDLFDRAVVLSHYIHNLS |
| | G129R- | SEMFSEFDKRYTHGRGFITKAINSCHTSSLA |
| | hPRL | TPEDKEQAQQMNQKDFLSLIVSILRSWNEPL |
| | (15-199) | YHLVTEVRGMQEAPEAILSKAVEIEEQTKRL |
| | | LERMELIVSQVHPETKENEIYPVWSGLPSLQ |
| | | MADEESRLSAYYNLLHCLRRDSHKIDNYLKL |
| | | LKCRIIHNNNC* |
| 7 | SA20- | MQRLIEDICLPRWGCLWEDDFAEAAAKEAAA |
| | A(EAAAK)₅A- | KEAAAKEAAAKEAAAKALRDLFDRAVVLSHY |
| | G129R- | IHNLSSEMFSEFDKRYTHGRGFITKAINSCH |
| | hPRL | TSSLATPEDKEQAQQMNQKDFLSLIVSILRS |
| | (15-199) | WNEPLYHLVTEVRGMQEAPEAILSKAVEIEE |
| | | QTKRLLERMELIVSQVHPETKENEIYPVWSG |
| | | LPSLQMADEESRLSAYYNLLHCLRRDSHKID |
| | | NYLKLLKCRIIHNNNC* |
| 8 | SA20- | MQRLIEDICLPRWGCLWEDDFAEAAAKEAAA |
| | [A(EAAAK)₄ | KEAAAKEAAAKAAEAAAKEAAAKEAAAKEAA |
| | A]₂-G129R- | AKALRDLFDRAVVLSHYIHNLSSEMFSEFDK |
| | hPRL | RYTHGRGFITKAINSCHTSSLATPEDKEQAQ |
| | (15-199) | QMNQKDFLSLIVSILRSWNEPLYHLVTEVRG |
| | | MQEAPEAILSKAVEIEEQTKRLLERMELIVS |
| | | QVHPETKENEIYPVWSGLPSLQMADEESRLS |
| | | AYYNLLHCLRRDSHKIDNYLKLLKCRIIHNN |
| | | NC* |
| 9 | SA20- | MQRLIEDICLPRWGCLWEDDFAEAAAKEAAA |
| | (A(EAAAK)₄ | KEAAAKEAAAKALEAEAAAKEAAAKEAAAKE |
| | ALEA(EAAA | AAAKALRDLFDRAVVLSHYIHNLSSEMFSEF |
| | K)₄A)- | DKRYTHGRGFITKAINSCHTSSLATPEDKEQ |
| | G129R- | AQQMNQKDFLSLIVSILRSWNEPLYHLVTEV |
| | hPRL | RGMQEAPEAILSKAVEIEEQTKRLLERMELI |
| | (15-199) | VSQVHPETKENEIYPVWSGLPSLQMADEESR |
| | | LSAYYNLLHCLRRDSHKIDNYLKLLKCRIIH |
| | | NNNC* |
| 10 | SA20- | MQRLIEDICLPRWGCLWEDDFEAAAKEAAAK |
| | (EAAAK)₃- | EAAAKLSRLFDHAMLQAHRAHQLAIDTYQEF |
| | G120R-hPL | EETYIPKDQKYSFLHDSQTSFCFSDSIPTPS |
| | (6-191) | NMEETQQKSNLELLRISLLLIESWLEPVRFL |
| | | RSMFANNLVYDTSDSDDYHLLKDLEERIQTL |
| | | MGRLEDGSRRTGQILKQTYSKFDTNSHNHDA |
| | | LLKNYGLLYCFRKDMDKVETFLRMVQCRSVE |
| | | GSCGF* |
| 11 | SA20- | MQRLIEDICLPRWGCLWEDDFEAAAKEAAAK |
| | (EAAAK)₃- | EAAAKLSRLFDNAMLRAHRLHQLAFDTYQEF |
| | G120R- | EEAYIPKEQKYSFLQNPQTSLCFSESIPTPS |
| | hGH | NREETQQKSNLELLRISLLLIQSWLEPVQFL |
| | (6-191) | RSVFANSLVYGASDSNVYDLLKDLEERIQTL |
| | | MGRLEDGSPRTGQIFKQTYSKFDTNSHNDDA |
| | | LLKNYGLLYCFRKDMDKVETFLRIVQCRSVE |
| | | GSCGF* |

### Serum Albumin Binding Polypeptides

Serum albumin is the most abundant protein in blood with an average serum half-life of three weeks in humans. Interestingly, serum albumin is able to bind the neonatal Fc Receptor (FcRn), which is broadly expressed and can protect against lysosomal degradation. Thus, without wishing to be bound by any one theory, it is understood that inclusion of serum albumin binding polypeptides in fusion polypeptides of the present disclosure can facilitate association with serum albumin, and as such, the FcRn, which can improve the in vivo half-life of fusion polypeptides of the present disclosure.

Serum albumin binding polypeptides of the present disclosure are or comprise polypeptides that specifically bind to serum albumin. Serum albumin can include, for example, human serum albumin, bovine serum albumin, and fetal bovine serum albumin. In some embodiments, the serum albumin is human serum albumin.

In some embodiments, serum albumin binding polypeptides of the present disclosure specifically bind to serum albumin at an epitope on the serum albumin that does not participate in the interaction of serum albumin with FcRn and as such, does not substantially interfere with, inhibit, prevent, and/or otherwise reduce binding of serum albumin to FcRn. In some such embodiments, serum albumin binding polypeptides of the present disclosure specifically bind to amino acid residues in domain IIa and/or domain IIb of serum albumin.

In some embodiments, serum albumin binding polypeptides (e.g., as part of a fusion polypeptide described herein) binds to serum albumin under physiological conditions (e.g., in a manner that is essentially independent of pH).

Further, the binding affinity of serum albumin binding polypeptides of the present disclosure to serum albumin can be selected to engineer a fusion polypeptide with a particular in vivo half-life. Accordingly, in some embodiments, a serum albumin binding polypeptide (e.g., of a fusion polypeptide) of the present disclosure has a binding affinity (K_{D}) to serum albumin of less than about 200 nM. In some embodiments, a serum albumin binding polypeptide of the present disclosure has a K_{D} to serum albumin of about 1 nM to about 200 nM, about 1 nM to about 150 nM, about 1 nM to about 100 nM, about 1 nM to about 75 nM, about 1 nM to about 50 nM, about 10 nM to about 200 nM, about 10 nM to about 150 nM, about 10 nM to about 100 nM, about 10 nM to about 75 nM, about 10 nM to about 50 nM, about 50 nM to about 200 nM, about 50 nM to about 150 nM, or about 50 nM to about 100 nM. In some embodiments, a serum albumin binding polypeptide of the present disclosure has a Kp to serum albumin of about 1 nM to about 10 nM. In some embodiments, a serum albumin binding polypeptide of the present disclosure has a Kp to serum albumin of about 10 nM to about 100 nM. In some embodiments, a serum albumin binding polypeptide of the present disclosure has a Kp to serum albumin of about 100 nM to about 200 nM.

Serum albumin binding polypeptides of the present disclosure may comprise, for example, albumin binding peptides, albumin binding domains from natural albumin binding proteins (e.g., from bacteria), and antibody fragments that bind to serum albumin, or functional fragments thereof that are known and available in the art. It is well within the level of the skilled artisan to use and/or modify such known serum albumin binding polypeptides for use in accordance with technologies of the present disclosure.

Examples of amino acid sequences of serum albumin binding polypeptides of the present disclosure are shown in Table 3. In some embodiments, the serum albumin binding polypeptide of the present disclosure comprises an amino acid sequence at least 60%, at least 65%, at least 70%, at least 75%, at least 80%, at least 85%, at least 90%, at least 95%, at least 96%, at least 97%, at least 98%, or at least 99% identical to any one of SEQ ID NOs: 12-46. In some embodiments, the serum albumin binding polypeptide of the present disclosure is or comprises the amino acid sequence of any one of SEQ ID NOs: 12-46 or a variant thereof.

TABLE 3
Examples of amino acid sequences of serum albumin binding (SAB)
polypeptides.

| SEQ | | |
|---|---|---|
| ID | | |
| NO: | Polypeptide | Amino Acid Sequence |
| 12 | SAB1 | DXCLPXWGCLW, where X can be any amino acid |
| 13 | SAB2 | DICLPRWGCLW |
| 14 | SAB3 | MQRLIEDICLPRWGCLWEDDF |
| 15 | SAB4 | LAX₃AKX₆X₇ANX₁₀ELDX₁₄YGVSDFYKRLIX₂₆KAK |
| | | TVEGVEALKX₃₉X₄₀ILX₄₃X₄₄LP, where X₃ is |
| | | selected from E, S, Q and C; X₆ is se- |
| | | lected from E, S and C; X₇ is selected |
| | | from A and S; X₁₀ is selected from A, S |
| | | and R; X₁₄ is selected from A, S, C and |
| | | K; X₂₆ is selected from D and E; X₃₉ is |
| | | selected from D and E; X₄₀ is selected |
| | | from A and E; X₄₃ is selected from A and |
| | | K; X₄₄ is selected from A, S and E; L in |
| | | position 45 is present or absent; and P |
| | | in position 46 is present or absent; an |
| | | S directly preceding the N-terminus; and |
| | | a K following the C-terminus |
| 16 | SAB5 | LKEAKEKAIEELKKAGITSDX₁X₂FDLINKAX₃X₄VE |
| | | GVNX₅LKDX₆ILKA, where X₁, X₂, X₃, X₄, |
| | | X₅, and X₆ can be any amino acid or a |
| | | subset of certain amino acids; and an |
| | | extension of 5 amino acids (such as |
| | | TIDEWL (SEQ ID NO: 124)) at its N- |
| | | terminus to improve stability |
| 17 | Cysteine-constrained | QRLIEDICLPRWGCLWEDDF |
| | synthetic peptide | |
| | SA20, SEQ 405 | |
| | (US20160185874A1) | |
| 18 | Unconstrained peptide | DTAVYYCNAAASYSDYDVFGGGTDFGPWGQGTQ |
| | 17D12, SEQ 1 | V |
| | (WO2008068280A1) | |
| 19 | Unconstrained peptide | AARYWDFDVFGGGTPVGG |
| | 59F2, SEQ 149 | |
| | (WO2009127691A1) | |
| 20 | Unconstrained peptide | AARDWDFDVFGGGTPVGG |
| | 59C2, SEQ 156 | |
| | (WO2009127691A1) | |
| 21 | Unconstrained peptide | AARSWDFDVFGGGTPVGG |
| | 59H12, SEQ 155 | |
| | (WO2009127691A1) | |
| 22 | Unconstrained peptide | YWWERRDWDFDVFGGGTP |
| | 80B10, SEQ 56 | |
| | (WO2011095545A1) | |
| 23 | Unconstrained peptide | WWEQDRDWDFDVFGGGTP |
| | 89D03, SEQ 72 | |
| | (WO2011095545A1) | |
| 24 | Unconstrained peptide | WWEQDRDWDFDVFGGGTPGGGGGGGGGSWWE |
| | Tandem 89D03 | QDRDWDFDVFGGGTP |
| | (WO2011095545A1) | |
| 25 | Natural albumin-binding | LAEAKVLANRELDKYGVSDYYKNLINNAKTVEGV |
| | polypeptide | KALIDEILAALP |
| | G148-GA3 | |
| 26 | Natural albumin-binding | TIDQWLLKNAKEDAIAELKKAGITSDFYFNAINKA |
| | polypeptide | KTVEEVNALKNEILKAHA |
| | ALB8-GA | |
| 27 | Natural albumin-binding | DITGAALLEAKEAAINELKQYGISDYYVTLINKAK |
| | polypeptide | TVEGVNALKAEILSALP |
| | ZAG-GA Module | |
| 28 | Natural albumin-binding | ALKAAALAKAKADAIEILKKYGIGDYYIKLINNGK |
| | polypeptide | TAEGVTALKDEILASLP |
| | MAG-GA Module | |
| 29 | GA synthetic variant | LAQAKEAAIKELKQYGIGDYYIKLINNAKTVEGVE |
| | PSD-1, SEQ 34 | SLKNEILKALP |
| | (US 10858430) | |
| 30 | GA synthetic variant | LKEAKEKAIEELKKAGITSDYYFDLINKAKTVEGV |
| | ABDCon | NALKDEILKA |
| 31 | G148-GA3 variant | LAEAKVLANRELDKYGVSDYYKNIINRAKTVEGV |
| | ABD00260 (US8937153) | RALKLHILAALP |
| 32 | G148-GA3 variant | LAEAKVLANRELDKYGVSDYYKNLINKAKTVEGV |
| | ABD00310 (US8937153) | EALTLHILAALP |
| 33 | G148-GA3 variant | LAEAKVLANRELDKYGVSDFYKRLINKAKTVEGV |
| | ABD00496 (US8937153) | EALKLHILAALP |
| 34 | G148-GA3 variant for | LAEAKEDAIKELDKYGVSDYYKRLISKAKTVEGV |
| | fusions | KALISEILAALP |
| | SEQ 122 (US 9593154) | |
| 35 | G148-GA3 variant for | LAEAKEDAIKELDKYGVSDYYKNLINKAKTVEGV |
| | fusions | EALTLHILAALP |
| | SEQ 123 (US 9593154) | |
| 36 | G148-GA3 variant for | LAEAKEDAIKELDKYGVSDYYKNLINKAKTVEGV |
| | fusions | EALISEILAALP |
| | SEQ 124 (US 9593154) | |
| 37 | G148-GA3 variant w/ | LAEAKEAANAELDSYGVSDFYKRLIDKAKTVEGV |
| | reduced immunogenicity | EALKDAILAALP |
| | SEQ 13 (US 9211344) | |
| 38 | ZAG-GA variant w/ | DITGAALLEAKDAAINELKQYGISDYYVHLINKAD |
| | reduced immunogenicity | TVEGVNALKDEILSALP |
| | SEQ 31 (US 9399678) | |
| 39 | Synthetic Nanofitin | VKVKFVFSGEEKEVDTSKIKWVLRWGKAVGFKY |
| | SEQ 55 (WO2022171852) | DDNGKIGYGFVAEKDAPKELLDMLARAEREK |
| 40 | Synthetic Nanofitin | VKVKFWPRGEEKVVDTSKIAWVLRADKTVMFKY |
| | B11, SEQ 58 | DDNGKKGYGVVLEKDAPKELLDMLARAEREK |
| | (WO2022171852) | |
| 41 | Synthetic Nanofitin | VKVKFVFSGEEKEVETSKIKWVLRWGKAVGFKYD |
| | SEQ 62 (WO2022171852) | DQGKIGYGFVAEKDAPKELLDLLARAEREK |
| 42 | Synthetic Nanofitin | VKVKFWPRGEEKVVETSKIAWVLRADKTVMFKY |
| | F06, SEQ 65 | DDQGKKGYGVVLEKDAPKELLDLLARAEREK |
| | (WO2022171852) | |
| 43 | Nanobody | QVQLVESGGGLVQPGGSLRLSCAASGFTFSNYWM |
| | SEQ 113 (US7807162) | YWVRVAPGKGLERISRDISTGGGYSYYADSVKGR |
| | | FTISRDNAKNTLYLQMNSLKPEDTALYYCAKDRE |
| | | AQVDTLDFDYRGQGTQVTVSS |
| 44 | Nanobody | EVQLVESGGGLVQPGGSLRLSCAASGFTFSSFGMS |
| | Humanized anti-HAS | WVRQAPGKEPEWVSSISGSGSDTLYADSVKGRFTI |
| | SEQ 115 (US7807162) | SRDNAKTTLYLQMNSLKPEDTAVYYCTIGGSLSRS |
| | | SQGTQVTVSS |
| 45 | Nanobody | EVQLVESGGGLVQPGNSLRLSCAASGFTFSSFGMS |
| | ALB-8, SEQ 33 | WVRQAPGKGLEWVSSISGSGSDTLYADSVKGRFTI |
| | (US8629244) | SRDNAKTTLYLQMNSLRPEDTAVYYCTIGGSLSRS |
| | | SQGTLVTVSS |
| 46 | Nanobody | EVQLVESGGGLVQPGNSLRLSCAASGFTFSKFGMS |
| | 10G, SEQ 9 (US9920115) | WVRQAPGKGLEWVSSISGSGRDTLYADSVKGRFTI |
| | | SRDNAKTTLYLQMNSLRPEDTAVYYCTIGGSLSVS |
| | | SQGTLVTVSS |

### Polypeptide Hormone Analogs

Polypeptide hormone analogs of the present disclosure are or comprise polypeptide hormone (e.g., wild-type GH, PL, or PRL) that share one or more particular structural features, elements, components, or moieties with a reference (e.g., a polypeptide hormone), but also differ in certain discrete ways (e.g., includes variants that render the analog capable of antagonizing its receptor activation, variants that increase pharmacological effect, N-terminal truncations that facilitate maintenance or improvement of antagonistic activity when part of a fusion polypeptide as described herein, and/or reduce partial agonism activity).

Polypeptide hormone analogs of the present disclosure can specifically bind to and antagonize their receptor (e.g., prolactin receptor, growth hormone receptor).

Polypeptide hormone analogs of the present disclosure can be or comprise a prolactin (PRL) analog, a growth hormone (GH) analog, or a placental lactogen (PL) analog. As GH and PL are endogenously agonists of GHR and PRL, GH, and PL are endogenously agonists of PRLR, polypeptide hormone analogs of the present disclosure can comprise variants which render the polypeptide hormone analogs capable of antagonizing its receptor. For example, G129R-PRL is an analog of polypeptide hormone, prolactin, capable of antagonizing the prolactin receptor, which comprises an amino acid substitution from glycine (G) at position 129 with arginine (R). Similarly, G120R-PL and G120R-GH are analogs of polypeptide hormones, placental lactogen and growth hormone, respectively, which render the analogs capable of antagonizing their receptor(s). Additional polypeptide hormone analogs capable of antagonizing its receptor activation are readily known in the art and appropriate use of such analogs is well within the level of the skilled artisan. Such polypeptide hormone analogs include, for example, G120R/K-hGH and S179D-hPRL. See, e.g., Goffin et al. Endocrine Rev. 2005, 26, 400-422.

In some embodiments, polypeptide hormone analogs of the present disclosure are N-terminally truncated relative to its parental polypeptide hormone. In some embodiments, a polypeptide hormone analog comprises a 1-20 amino acid N-terminal truncation relative to its parental polypeptide hormone. In some embodiments, a polypeptide hormone analog comprises a 1 amino acid, 2 amino acid, 3 amino acid, 4 amino acid, 5 amino acid, 6 amino acid, 7 amino acid, 8 amino acid, 9 amino acid, 10 amino acid, 11 amino acid, 12 amino acid, 13 amino acid, 14 amino acid, 15 amino acid, 16 amino acid, 17 amino acid, 18 amino acid, 19 amino acid, or 20 amino acid N-terminal truncation relative to its parental polypeptide hormone. In some embodiments, a polypeptide hormone analog comprises a 10 amino acid N-terminal truncation relative to its parental polypeptide hormone. In some embodiments, a polypeptide hormone analog comprises a 11 amino acid N-terminal truncation relative to its parental polypeptide hormone. In some embodiments, a polypeptide hormone analog comprises a 12 amino acid N-terminal truncation relative to its parental polypeptide hormone. In some embodiments, a polypeptide hormone analog comprises a 13 amino acid N-terminal truncation relative to its parental polypeptide hormone. In some embodiments, a polypeptide hormone analog comprises a 14 amino acid N-terminal truncation relative to its parental polypeptide hormone. In some embodiments, a polypeptide hormone analog comprises a 15 amino acid N-terminal truncation relative to its parental polypeptide hormone. In some embodiments, a polypeptide hormone analog comprises a 16 amino acid N-terminal truncation relative to its parental polypeptide hormone. In some embodiments, a polypeptide hormone analog comprises a 17 amino acid N-terminal truncation relative to its parental polypeptide hormone. In some embodiments, a polypeptide hormone analog comprises a 18 amino acid N-terminal truncation relative to its parental polypeptide hormone.

Without wishing to be bound by any one theory, it is hypothesized that such N-terminal truncation of polypeptide hormone analogs of the present disclosure facilitates maintenance or improvement of the functional activity (e.g., antagonist activity) of a polypeptide hormone analog as part of a fusion polypeptide described herein. As such, the present disclosure also provides methods of maintaining or improving functional activity of a polypeptide hormone analog in a fusion polypeptide, comprising N-terminally truncating the polypeptide hormone analog relative to the parental polypeptide hormone. In some embodiments, maintained functional activity is a functional (e.g., antagonist) activity of about 80%, about 85%, about 90%, about 95%, about 100%, about 105%, about 110%, about 115%, or about 120% relative to an appropriate reference (e.g., a parental polypeptide hormone, a non-N-terminally truncated polypeptide hormone analog). In some embodiments, improved functional activity is a functional (e.g., antagonist) activity of about 125%, about 130%, about 140%, about 150%, about 160%, about 170%, about 180%, about 190%, about 200%, about 210%, about 225%, about 250%, about 275%, or about 300% relative to an appropriate reference (e.g., a parental polypeptide hormone, a non-N-terminally truncated polypeptide hormone analog).

A polypeptide hormone analog of varying levels of affinity to its receptor (e.g., GHR, PRLR) may be useful in particular contexts. In some embodiments, a polypeptide hormone analog (e.g., of a fusion polypeptide) of the present disclosure has a binding affinity (K_{D}) to its receptor of less than about 100 nM. In some embodiments, a polypeptide hormone analog of the present disclosure has a K_{D} to its receptor of about 0.01 nM to about 100 nM, about 0.01 nM to about 90 nM, about 0.01 nM to about 80 nM, about 0.01 nM to about 70 nM, about 0.01 nM to about 60 nM, about 0.01 nM to about 50 nM, about 0.1 nM to about 100 nM, about 0.1 nM to about 90 nM, about 0.1 nM to about 80 nM, about 0.1 nM to about 70 nM, about 0.1 nM to about 60 nM, or about 0.1 nM to about 50 nM. In some embodiments, a polypeptide hormone analog of the present disclosure has a Kp to its receptor of about 0.01 nM to about 10 nM. In some embodiments, a polypeptide hormone analog of the present disclosure has a Kp to its receptor of about 10 nM to about 50 nM. In some embodiments, a polypeptide hormone analog of the present disclosure has a K_{D} to its receptor of about 50 nM to about 100 nM.

In some embodiments, a polypeptide hormone analog of the present disclosure is a prolactin (PRL) analog. In some embodiments, a PRL analog comprises an amino acid sequence at least 60%, at least 65%, at least 70%, at least 75%, at least 80%, at least 85%, at least 90%, at least 95%, at least 96%, at least 97%, at least 98%, or at least 99% identical to any one of SEQ ID NOs: 47-51. In some embodiments, a PRL analog comprises an amino acid sequence of any one of SEQ ID NOs: 47-51 or a variant thereof.

In some embodiments, the polypeptide hormone analog is a growth hormone (GH) analog. In some embodiments, a GH analog comprises an amino acid sequence at least 60%, at least 65%, at least 70%, at least 75%, at least 80%, at least 85%, at least 90%, at least 95%, at least 96%, at least 97%, at least 98%, or at least 99% identical to SEQ ID NO: 54 or 55. In some embodiments, a GH analog comprises an amino acid sequence of SEQ ID NO: 54 or SEQ ID NO: 55 or a variant thereof.

In some embodiments, the polypeptide hormone analog is a placental lactogen (PL) analog. In some embodiments, a PL analog comprises an amino acid sequence at least 60%, at least 65%, at least 70%, at least 75%, at least 80%, at least 85%, at least 90%, at least 95%, at least 96%, at least 97%, at least 98%, or at least 99% identical to SEQ ID NO: 52 or 53. In some embodiments, a PL analog comprises an amino acid sequence of SEQ ID NO: 52 or SEQ ID NO: 53 or a variant thereof.

Examples of amino acid sequences of polypeptide hormone analogs (e.g., N-terminally truncated analogs) capable of antagonizing its receptor activation are shown in Table 4.

TABLE 4
Examples of amino acid sequences of
polypeptide hormone analogs

| SEQ | | |
|---|---|---|
| ID | | |
| NO: | Polypeptide | Amino Acid Sequence |
| 47 | G129R-hPRL | LRDLFDRAVVLSHYIHNLSSEMFSEFDKRYTHG |
| | (15-199) | RGFITKAINSCHTSSLATPEDKEQAQQMNQKDFL |
| | | SLIVSILRSWNEPLYHLVTEVRGMQEAPEAILSK |
| | | AVEIEEQTKRLLERMELIVSQVHPETKENEIYPV |
| | | WSGLPSLQMADEESRLSAYYNLLHCLRRDSHKI |
| | | DNYLKLLKCRIIHNNNC* |
| 48 | G129K-hPRL | LRDLFDRAVVLSHYIHNLSSEMFSEFDKRYTHG |
| | (15-199) | RGFITKAINSCHTSSLATPEDKEQAQQMNQKDFL |
| | | SLIVSILRSWNEPLYHLVTEVRGMQEAPEAILSK |
| | | AVEIEEQTKRLLEKMELIVSQVHPETKENEIYPV |
| | | WSGLPSLQMADEESRLSAYYNLLHCLRRDSHKI |
| | | DNYLKLLKCRIIHNNNC* |
| 49 | S61A, D68N, | LRDLFDRAVVLSHYIHNLSSEMFSEFDKRYTHG |
| | Q73L, G129R, | RGFITKAINSCHTASLATPENKEQALQMNQKDF |
| | K190R-hPRL | LSLIVSILRSWNEPLYHLVTEVRGMQEAPEAILS |
| | (15-199) | KAVEIEEQTKRLLERMELIVSQVHPETKENEIYP |
| | | VWSGLPSLQMADEESRLSAYYNLLHCLRRDSH |
| | | KIDNYLKLLRCRIIHNNNC* |
| 50 | L25Q, H46R, | LRDLFDRAVVQSHYIHNLSSEMFSEFDKRYTRG |
| | T52A, I55F, | RGFIAKAFNSCHTSSLATPENKEQALQMNQKDF |
| | D68N, Q73L, | LSLIVSILRSWNEPLYHLVTEVRGMQEAPEAILS |
| | G129R, | KAVEIEEQTKRLLERMELIVSQVHPETKENEIYP |
| | K190R-hPRL | VWSGLPSLQMADEESRLSAYYNLLHCLRRDSH |
| | (15-199) | KIDNYLKLLRCRIIHNNNC* |
| 51 | E62S, D63N, | LRDLFDRAVVLSHYIHNLSSEMFSEFDKRYTHG |
| | Q66E, G129K, | RGFITKAINSCHTSSLATPSNKEEAQQMNQKDFL |
| | H171D, D174A, | SLIVSILRSWNEPLYHLVTEVRGMQEAPEAILSK |
| | N175T, Y176F, | AVEIEEQTKRLLEGMELIVSQVHPETKENEIYPV |
| | K178R-hPRL | WSGLPSLQMADEESRLSAYYNLLHCLRRDSDKI |
| | (15-199) | ATFLRLLKCRIIHNNNC* |
| 52 | G120R-hPL | LSRLFDHAMLQAHRAHQLAIDTYQEFEETYIPK |
| | (6-191) | DQKYSFLHDSQTSFCFSDSIPTPSNMEETQQKSN |
| | | LELLRISLLLIESWLEPVRFLRSMFANNLVYDTS |
| | | DSDDYHLLKDLEERIQTLMGRLEDGSRRTGQIL |
| | | KQTYSKFDTNSHNHDALLKNYGLLYCFRKDMD |
| | | KVETFLRMVQCRSVEGSCGF* |
| 53 | D56E, M64R, | LSRLFDHAMLQAHRAHQLAIDTYQEFEETYIPK |
| | G120R, E174A, | DQKYSFLHDSQTSFCFSESIPTPSNREETQQKSNL |
| | M179I-hPL | ELLRISLLLIESWLEPVRFLRSMFANNLVYDTSDS |
| | (6-191) | DDYHLLKDLEERIQTLMGRLEDGSRRTGQILKQ |
| | | TYSKFDTNSHNHDALLKNYGLLYCFRKDMDKV |
| | | ATFLRIVQCRSVEGSCGF* |
| 54 | G120R-hGH | LSRLFDNAMLRAHRLHQLAFDTYQEFEEAYIPK |
| | (6-191) | EQKYSFLQNPQTSLCFSESIPTPSNREETQQKSNL |
| | | ELLRISLLLIQSWLEPVQFLRSVFANSLVYGASDS |
| | | NVYDLLKDLEERIQTLMGRLEDGSPRTGQIFKQT |
| | | YSKFDTNSHNDDALLKNYGLLYCFRKDMDKVE |
| | | TFLRIVQCRSVEGSCGF |
| 55 | H18D, H21N, | LSRLFDNAMLRADRLNQLAFDTYQEFEEAYIPK |
| | G120K, R167N, | EQKYSFLQNPQTSLCFSESIPTPSNREETQQKSNL |
| | K168A, D171S, | ELLRISLLLIQSWLEPVQFLRSVFANSLVYGASDS |
| | K172R,E174S, | NVYDLLKDLEEKIQTLMGRLEDGSPRTGQIFKQ |
| | I179T-hGH | TYSKFDTNSHNDDALLKNYGLLYCFNADMSRV |
| | (6-191) | STFLRTVQCRSVEGSCGF* |

### Linkers

Linkers of the present disclosure are polypeptide linkers and refer to a portion (e.g., a polypeptide portion) of a fusion polypeptide that adjoins (i) a serum albumin binding polypeptide and (ii) a polypeptide hormone analog to one another, wherein the (i) is N-terminal to the linker and (ii) is C-terminal to the linker.

In some embodiments, a polypeptide linker is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more amino acids in length.

In some embodiments, a linker is characterized in that it tends not to adopt a rigid three-dimensional structure, but rather provides flexibility to a fusion polypeptide (referred to herein as a “flexible linker”).

In some embodiments, a linker is characterized in that it tends to adopt a rigid three-dimensional structure (e.g., a rigid alpha helical structure) and provides a more defined structure to a fusion polypeptide (referred to herein as a “rigid linker”).

In some embodiments, fusion polypeptides of the present disclosure comprise a linker that is a rigid alpha-helical linker. In some embodiments, a rigid alpha-helical linker comprises an amino acid sequence of any one of SEQ ID NOs: 56-87. Examples of amino acid sequences of rigid linkers (e.g., alpha helical rigid linkers) are shown in Table 5. Without wishing to be bound by any one theory, it is hypothesized that inclusion of a rigid linker in a fusion polypeptide of the present disclosure facilitates a polypeptide hormone analog to interact (e.g., antagonize) its receptor (e.g., while maintaining potency of the polypeptide hormone analog) without sterically hindering the interaction of the serum albumin binding polypeptide with serum albumin.

In some embodiments, fusion polypeptides of the present disclosure further comprise a second linker. A second linker can be either of a rigid linker or a flexible linker. In some embodiments, a second linker is a rigid linker. In some embodiments, a second linker is a flexible linker. In some embodiments, a flexible linker comprises an amino acid sequence of any one of SEQ ID NOs: 88-109. Examples of amino acid sequences of flexible linkers are shown in Table 6.

TABLE 5
Examples of amino acid sequences of rigid
linkers.

| SEQ | | |
|---|---|---|
| ID | Poly- | |
| NO: | peptide | Amino Acid Sequence |
| 56 | (EAAAK)₁ | EAAAK |
| 57 | (EAAAK)₂ | EAAAKEAAAK |
| 58 | (EAAAK)₃ | EAAAKEAAAKEAAAK |
| 59 | (EAAAK)₄ | EAAAKEAAAKEAAAKEAAAK |
| 60 | (EAAAK)₅ | EAAAKEAAAKEAAAKEAAAKEAAAK |
| 61 | (EAAAK)₆ | EAAAKEAAAKEAAAKEAAAKEAAAKEAAAK |
| 62 | (EAAAK)₇ | EAAAKEAAAKEAAAKEAAAKEAAAKEAAAK |
| | | EAAAK |
| 63 | (EAAAK)₈ | EAAAKEAAAKEAAAKEAAAKEAAAKEAAAK |
| | | EAAAKEAAAK |
| 64 | (EAAAK)₉ | EAAAKEAAAKEAAAKEAAAKEAAAKEAAAK |
| | | EAAAKEAAAKEAAAK |
| 65 | (EAAAK)₁₀ | EAAAKEAAAKEAAAKEAAAKEAAAKEAAAK |
| | | EAAAKEAAAKEAAAKEAAAK |
| 66 | A(EAAK)₁A | AEAAKA |
| 67 | A(EAAK)₂A | AEAAAKEAAAKA |
| 68 | A(EAAK)₃A | AEAAAKEAAAKEAAAKA |
| 69 | A(EAAK)₄A | AEAAAKEAAAKEAAAKEAAAKA |
| 70 | A(EAAK)₅A | AEAAAKEAAAKEAAAKEAAAKEAAAKA |
| 71 | A(EAAK)₆A | AEAAAKEAAAKEAAAKEAAAKEAAAKEAAA |
| | | KA |
| 72 | A(EAAK)₇A | AEAAAKEAAAKEAAAKEAAAKEAAAKEAAA |
| | | KEAAAKA |
| 73 | A(EAAK)₈A | AEAAAKEAAAKEAAAKEAAAKEAAAKEAAA |
| | | KEAAAKEAAAKA |
| 74 | A(EAAK)₉A | AEAAAKEAAAKEAAAKEAAAKEAAAKEAAA |
| | | KEAAAKEAAAKEAAAKA |
| 75 | A(EAAK)₁₀A | AEAAAKEAAAKEAAAKEAAAKEAAAKEAAA |
| | | KEAAAKEAAAKEAAAKEAAAKA |
| 76 | [A(EAAAK)₄A]₂ | AEAAAKEAAAKEAAAKEAAAKAAEAAAKEA |
| | | AAKEAAAKEAAAKA |
| 77 | A(EAAAK)₄ALE | AEAAAKEAAAKEAAAKEAAAKALEAEAAAK |
| | A(EAAAK)₄A | EAAAKEAAAKEAAAKA |
| | (XP)₁ | XP, where X is any amino acid, |
| | | preferably selected from |
| | | alanine, lysine and glutamine |
| | (XP)₂ | XPXP, where X is any amino |
| | | acid, preferably selected from |
| | | alanine, lysine and glutamine |
| | (XP)₃ | XPXPXP, where X is any amino |
| | | acid, preferably selected |
| | | from alanine, lysine and |
| | | glutamine |
| 81 | (XP)₄ | XPXPXPXP, where X is any amino |
| | | acid, preferably selected from |
| | | alanine, lysine and glutamine |
| 82 | (XP)₅ | XPXPXPXPXP, where X is any amino |
| | | acid, preferably selected from |
| | | alanine, lysine and glutamine |
| 83 | (XP)₆ | XPXPXPXPXPXP, where X is any |
| | | amino acid, preferably selected |
| | | from alanine, lysine and |
| | | glutamine |
| 84 | (XP)₇ | XPXPXPXPXPXPXP, where X is any |
| | | amino acid, preferably selected |
| | | from alanine, lysine and |
| | | glutamine |
| 85 | (XP)₈ | XPXPXPXPXPXPXPXP, where X is any |
| | | amino acid, preferably selected |
| | | from alanine, lysine and |
| | | glutamine |
| 86 | (XP)₉ | XPXPXPXPXPXPXPXPXP, where X is |
| | | any amino acid, preferably |
| | | selected from alanine, lysine |
| | | and glutamine |
| 87 | (XP)₁₀ | XPXPXPXPXPXPXPXPXPXP, where X is |
| | | any amino acid, preferably |
| | | selected from alanine, lysine and |
| | | glutamine |

TABLE 6
Examples of amino acid sequences of
flexible linkers.

| SEQ | | |
|---|---|---|
| ID | | |
| NO: | Polypeptide | Amino Acid Sequence |
| 88 | (GGGGS)₃ | GGGGSGGGGSGGGGS |
| 89 | (GGGGS)₄ | GGGGSGGGGSGGGGSGGGGS |
| | (GS)₁ | GS |
| 91 | (GS)₂ | GSGS |
| 92 | (GS)₃ | GSGSGS |
| 93 | (GS)₄ | GSGSGSGS |
| 94 | (GS)₅ | GSGSGSGSGS |
| 95 | (GS)₆ | GSGSGSGSGSGS |
| 96 | (GS)₇ | GSGSGSGSGSGSGS |
| 97 | (GS)₈ | GSGSGSGSGSGSGS |
| 98 | (GS)₉ | GSGSGSGSGSGSGSGS |
| 99 | (GS)₁₀ | GSGSGSGSGSGSGSGS |
| 100 | (GSGGS)₁ | GSGGS |
| 101 | (GSGGS)₂ | GSGGSGSGGS |
| 102 | (GSGGS)₃ | GSGGSGSGGSGSGGS |
| 103 | (GSGGS)₄ | GSGGSGSGGSGSGGSGSGGS |
| 104 | (GSGGS)₅ | GSGGSGSGGSGSGGSGSGGSGSGGS |
| 105 | (GGGS)₁ | GGGS |
| 106 | (GGGS)₂ | GGGSGGGS |
| 107 | (GGGS)₃ | GGGSGGGSGGGS |
| 108 | (GGGS)₄ | GGGSGGGSGGGSGGGS |
| 109 | (GGGS)₅ | GGGSGGGSGGGSGGGSGGGS |

### Variants

Fusion polypeptides, serum albumin binding polypeptides, and polypeptide hormone analogs as described herein can be a variant of a relevant reference polypeptide (e.g., a wild-type polypeptide or a functional fragment thereof, fusion polypeptides, serum albumin binding polypeptides, or polypeptide hormone analog described herein).

In some embodiments, a variant (e.g., a variant fusion polypeptide, a variant serum albumin binding polypeptide, a variant polypeptide hormone analog) shows at least 70% identity to a reference polypeptide (e.g., a fusion polypeptide, a serum albumin binding polypeptide, a polypeptide hormone analog as described herein). In some such embodiments, a variant shows at least 70%, 75%, 80%, 85%, 90%, 91%, 92%, 93%, 94%, 95%, 96%, 97%, 98%, 99% or higher identity to a reference polypeptide.

In some embodiments, a variant (e.g., a variant fusion polypeptide, a variant serum albumin binding polypeptide, a variant polypeptide hormone analog) comprises one or more non-conservative modifications (e.g., substitutions, deletions or additions) relative to a reference. Such non-conservative modifications may increase and/or decrease one or more functional characteristics of the fusion polypeptide (e.g., activity, such as signaling and/or affinity to serum albumin or its receptor) relative to a reference polypeptide.

In some embodiments, a variant (e.g., a variant fusion polypeptide, a variant serum albumin binding polypeptide, a variant polypeptide hormone analog) comprises one or more conservative or otherwise non-disruptive modifications (e.g., substitutions, deletions or additions) relative to its reference. In some embodiments, a variant (e.g., a variant fusion polypeptide, a variant serum albumin binding polypeptide, a variant polypeptide hormone analog) does not comprise any non-conservative (“disruptive”) modifications (e.g., substitutions, deletions or additions) relative to a reference. A variant that does not comprise any disruptive modifications can maintain one or more functional characteristics (e.g., maintains comparable activity, such as signaling and/or affinity to serum albumin or prolactin receptor) of a reference polypeptide. For example, a variant that does not comprise any disruptive modifications can maintain at least about 70%, 75%, 80%, 85%, 90%, 91%, 92%, 93%, 94%, 95%, 96%, 97%, 98%, 99% or higher activity compared to a reference polypeptide.

### Nucleic Acids Encoding Polypeptides

The present disclosure also provides nucleic acids encoding any of the polypeptides described herein (e.g., a serum albumin binding polypeptide, a polypeptide hormone analog, a linker, a fusion polypeptide). Nucleic acids encoding polypeptides of the present disclosure are recombinant polynucleotides, and can be either DNA or RNA. Such recombinant polynucleotides may be prepared by a variety of methods available and known in the art. For example, nucleic acids encoding a serum albumin binding polypeptide and/or polypeptide hormone analog of the present disclosure may be excised from DNA using restriction enzymes, may be amplified from plasmids or genomic polynucleotide sequences using, for example, polymerase chain reaction, or may be synthesized (e.g., using chemical synthesis techniques or in vitro transcription). Fusion polypeptides described herein can be designed at the amino acid level and back translated using a variety of software products known in the art. The nucleotide sequence is optimized for the desired expression host, e.g. based on restriction site content, codon optimization, and protein expression. A combination of known methods may also be utilized to prepare a nucleic acid encoding a polypeptide of the present disclosure.

Recombinant polynucleotides encoding a polypeptide of the disclosure may be cloned into a vector capable of expressing a polypeptide of the present disclosure. Cloning may be carried out according a variety of methods available, including, for example, isothermal assembly reaction, blunt-end cloning, recombination-based cloning, restriction digestion and ligation). A vector can be, for example, a viral vector, a non-viral vector, and/or a plasmid.

A plurality of expression vectors are available and known to those of skill in the art and can be used for expression of polypeptides provided herein. Examples of expression vectors for expression in E. coli include, for example, pET26b (Novagen), pcDNA5 (Invitrogen) for expression in mammalian cells, PICHIAPINK™ Yeast Expression Systems (Invitrogen), BACUVANCE™ Baculovirus Expression System (GenScript). The choice of expression vector can be influenced by the choice of host expression system. Such selection is well within the level of skill of the skilled artisan. In general, expression vectors can comprise transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector in the cells. Expression vectors can be designed in a modular manner by incorporating unique restriction sites at the end of the products prior to synthesis such that linker sequences can be easily cut out and changed, as known in the art.

Vectors also can contain additional nucleotide sequences operably linked to the ligated nucleic acid molecule, such as, for example, an epitope tag, such as for localization, e.g., a hexa-his tag (SEQ ID NO: 121) or a myc tag, hemagglutinin tag or a tag for purification, for example, a GST fusion, and/or a sequence for directing polypeptide localization (e.g., secretion and/or membrane association).

Expression of polypeptides described herein can be controlled by any promoter/enhancer known in the art. Suitable bacterial promoters are well known in the art and described herein below. Other suitable promoters for mammalian cells, yeast cells and insect cells are well known in the art. Selection of the promoter used to direct expression of a heterologous nucleic acid depends on the particular application and is within the level of skill of the skilled artisan. Promoters which can be used include but are not limited to eukaryotic expression vectors containing the SV40 early promoter (Bemoist and Chambon, Nature 290:304-310 (1981)), the promoter contained in the 3′ long terminal repeat of Rous sarcoma virus (Yamamoto et al., Cell 22:787-797 (1980)), the herpes thymidine kinase promoter (Wagner et al., Proc. Natl. Acad. Sci. USA 75:1441-1445 (1981)), the regulatory sequences of the metallothionein gene (Brinster et al., Nature 296:39-42 (1982)); prokaryotic expression vectors such as the b-lactamase promoter (Jay et al., Proc. Natl. Acad. Sci. USA 75:5543 (1981)) or the tac promoter (DeBoer et al., Proc. Natl. Acad. Sci. USA 50:21-25 (1983)); see also “Useful Proteins from Recombinant Bacteria”: in Scientific American 242:79-94 (1980)); plant expression vectors containing the nopaline synthetase promoter (Herrera-Estrella et al., Nature 505:209-213 (1984)) or the cauliflower mosaic virus 35S RNA promoter (Gardner et al., Nucleic Acids Res. 9:2871 (1981)), and the promoter of the photosynthetic enzyme ribulose bisphosphate carboxylase (Herrera-Estrella et al., Nature 510:1 15-120 (1984)); promoter elements from yeast and other fungi such as the Gal4 promoter, the alcohol dehydrogenase promoter, the phosphoglycerol kinase promoter, the alkaline phosphatase promoter, and the following animal transcriptional control regions that exhibit tissue specificity and have been used in transgenic animals: elastase I gene control region which is active in pancreatic acinar cells (Swift et al., Cell 55:639-646 (1984); Ornitz et al., Cold Spring Harbor Symp. Quant. Biol. 50:399-409 (1986); MacDonald, Hepatology 7:425-515 (1987)); insulin gene control region which is active in pancreatic beta cells (Hanahan et al., Nature 515:115-122 (1985)), immunoglobulin gene control region which is active in lymphoid cells (Grosschedl et al., Cell 55:647-658 (1984); Adams et al., Nature 515:533-538 (1985); Alexander et al., Mol. Cell Biol. 7:1436-1444 (1987)), mouse mammary tumor virus control region which is active in testicular, breast, lymphoid and mast cells (Leder et al., Cell 15:485-495 (1986)), albumin gene control region which is active in liver (Pinckert et al., Genes andDevel. 1:268-276 (1987)), alpha-fetoprotein gene control region which is active in liver (Krumlauf et al., Mol. Cell. Biol. 5:1639-403 (1985)); Hammer et al., Science 255:53-58 (1987)), alpha-1 antitrypsin gene control region which is active in liver (Kelsey et al., Genes andDevel. 7:161-171 (1987)), beta globin gene control region which is active in myeloid cells (Magram et al., Nature 515:338-340 (1985)); Kollias et al., Cell 5:89-94 (1986)), myelin basic protein gene control region which is active in oligodendrocyte cells of the brain (Readhead etal, Cell 15:703-712 (1987)), myosin light chain-2 gene control region which is active in skeletal muscle (Shani, Nature 514:283-286 (1985)), and gonadotrophic releasing hormone gene control region which is active in gonadotrophs of the hypothalamus (Mason et al, Science 254:1372-1378 (1986)).

In addition to the promoter, expression vectors typically contain a transcription unit or expression cassette that contains all the additional elements required for the expression of polypeptide in host cells. A typical expression cassette contains a promoter operably linked to the nucleic acid sequence encoding the polypeptide chains of interest and signals required for efficient polyadenylation of the transcript, ribosome binding sites and translation termination. Additional elements of the cassette can include enhancers. In addition, the cassette typically contains a transcription termination region downstream of the structural gene to provide for efficient termination. The termination region can be obtained from the same gene as the promoter sequence or can be obtained from different genes.

Examples of nucleic acids encoding fusion polypeptides of the present disclosure are shown in Table 7. In some embodiments, a nucleic acid encoding a fusion polypeptide of the present disclosure comprises a nucleotide sequence at least 70%, at least 75%, at least 80%, at least 85%, at least 90%, at least 95%, at least 96%, at least 97%, at least 98%, or at least 99% identical to the nucleic acid sequence of any one of SEQ ID NOs: 110-120. In some embodiments, a nucleic acid encoding a fusion polypeptide of the present disclosure comprises a nucleotide sequence of any one of SEQ ID NOs: 110-120.

TABLE 7
Examples of nucleic acids encoding fusion
polypeptides.

| SEQ | | |
|---|---|---|
| ID | Nucleic | |
| NO: | acid | Nucleic acid sequence |
| 110 | SA20- | ATGCAACGTCTTATCGAGGACATTTGCTTACCTCGGT |
| | (G4S)3- | GGGGCTGCTTATGGGAGGATGATTTTGGCGGCGGTG |
| | G129R- | GGTCCGGTGGTGGTGGCAGTGGTGGTGGCGGGAGCT |
| | hPRL | TACGCGACCTCTTTGACCGCGCGGTCGTGCTGAGCC |
| | (15-199) | ACTACATTCATAATCTGAGTTCCGAGATGTTTAGCGA |
| | | ATTTGATAAGCGCTACACGCATGGTCGCGGGTTTATT |
| | | ACGAAGGCCATTAATTCATGTCACACGAGTTCCTTG |
| | | GCGACGCCAGAGGACAAGGAACAAGCACAGCAAAT |
| | | GAATCAAAAGGACTTTTTATCCCTGATCGTGTCCATT |
| | | CTGCGGAGTTGGAACGAACCGCTTTACCACCTTGTG |
| | | ACCGAAGTCCGTGGTATGCAGGAAGCTCCTGAGGCG |
| | | ATCCTCTCCAAGGCAGTCGAAATTGAGGAACAAACC |
| | | AAACGGTTGTTGGAACGGATGGAGCTGATCGTATCA |
| | | CAAGTGCATCCTGAGACTAAAGAAAACGAAATCTAC |
| | | CCTGTTTGGTCGGGCCTTCCATCATTACAAATGGCTG |
| | | ACGAAGAGTCACGCCTTAGCGCGTACTACAACTTAC |
| | | TGCACTGCCTTCGGCGCGATTCCCATAAAATCGACA |
| | | ACTATCTCAAACTGCTCAAATGCCGGATCATCCATA |
| | | ACAATAATTGTTAA |
| 111 | SA20- | ATGCAGCGTTTAATTGAGGATATTTGTCTGCCTCGTT |
| | (G4S)4- | GGGGCTGTTTGTGGGAAGATGACTTCGGTGGCGGTG |
| | G129R- | GCTCCGGGGGGGGTGGTAGCGGTGGGGGTGGCTCCG |
| | hPRL | GTGGTGGTGGGAGTTTACGGGACTTATTTGATCGGG |
| | (15-199) | CTGTCGTGCTGTCTCACTATATCCATAATTTAAGTTC |
| | | CGAGATGTTTTCGGAATTTGACAAACGCTACACGCA |
| | | CGGTCGCGGTTTCATTACAAAGGCCATTAATAGCTG |
| | | CCACACATCGTCGTTAGCCACGCCTGAGGATAAGGA |
| | | ACAAGCCCAGCAAATGAACCAAAAGGACTTTCTGAG |
| | | TCTTATCGTATCGATCCTTCGGTCTTGGAACGAACCG |
| | | TTGTACCACCTCGTGACGGAGGTTCGTGGTATGCAA |
| | | GAAGCTCCGGAAGCCATTCTCTCCAAGGCTGTTGAG |
| | | ATCGAAGAACAGACGAAACGTCTCTTGGAACGTATG |
| | | GAGCTGATCGTTAGTCAAGTACATCCTGAGACAAAG |
| | | GAGAATGAAATTTATCCAGTTTGGTCGGGCCTTCCTT |
| | | CGCTCCAGATGGCTGATGAGGAAAGCCGCCTCTCGG |
| | | CCTATTATAATCTTCTTCACTGTTTGCGCCGCGATTC |
| | | ACATAAAATCGACAATTACTTAAAACTCCTGAAGTG |
| | | TCGGATCATTCATAACAACAACTGTTAA |
| 112 | SA20- | ATGCAGCGTCTCATTGAAGATATTTGTCTGCCGCGCT |
| | (EAAAK) | GGGGCTGCCTCTGGGAAGATGATTTTGAGGCTGCCG |
| | 3-G129R- | CCAAGGAGGCCGCAGCCAAAGAGGCTGCCGCTAAG |
| | hPRL | CTGCGGGACTTGTTTGACCGGGCGGTTGTATTGAGTC |
| | (15-199) | ACTACATTCATAACTTATCCTCCGAAATGTTTTCTGA |
| | | GTTCGATAAACGGTACACGCATGGCCGGGGGTTTAT |
| | | CACAAAGGCCATCAACTCATGCCATACCAGTAGCTT |
| | | GGCGACACCGGAGGATAAAGAGCAGGCTCAACAGA |
| | | TGAACCAGAAGGACTTCCTGAGCCTTATTGTTTCCAT |
| | | CTTGCGGTCTTGGAATGAACCATTATATCACTTAGTC |
| | | ACCGAGGTGCGTGGGATGCAAGAAGCGCCTGAGGC |
| | | GATTCTCAGTAAGGCCGTGGAAATCGAAGAACAGAC |
| | | CAAGCGTCTGCTTGAACGGATGGAACTTATTGTTTCG |
| | | CAAGTCCACCCGGAGACTAAGGAAAACGAAATTTAT |
| | | CCTGTCTGGTCGGGTCTTCCATCTTTACAAATGGCAG |
| | | ACGAAGAGTCGCGGCTTTCGGCGTATTATAACCTCTT |
| | | GCATTGCTTACGGCGGGATAGTCATAAGATTGACAA |
| | | CTACCTCAAATTACTTAAGTGCCGGATTATCCACAAC |
| | | AATAACTGTTAA |
| 113 | SA20- | ATGCAGCGTCTTATTGAGGATATCTGTTTACCTCGGT |
| | A(EAAAK) | GGGGCTGTCTCTGGGAGGATGACTTTGCCGAAGCTG |
| | 2A-G129R- | CTGCTAAAGAAGCCGCCGCAAAGGCCCTTCGGGATC |
| | hPRL | TGTTTGACCGGGCAGTTGTCTTGAGTCACTACATTCA |
| | (15-199) | TAATTTAAGCAGTGAGATGTTCTCAGAGTTCGATAA |
| | | GCGCTATACTCACGGCCGTGGTTTTATTACTAAGGCG |
| | | ATTAATTCATGTCACACGTCAAGTCTGGCTACACCTG |
| | | AGGATAAAGAGCAAGCCCAGCAGATGAATCAGAAG |
| | | GACTTTTTAAGTCTGATTGTGTCAATCCTGCGCTCTT |
| | | GGAATGAACCGCTGTATCATTTGGTCACTGAAGTCC |
| | | GCGGGATGCAAGAAGCACCTGAGGCTATCCTTAGTA |
| | | AGGCCGTTGAAATCGAAGAACAGACGAAGCGTCTTC |
| | | TGGAACGCATGGAACTGATTGTTAGCCAAGTTCATC |
| | | CTGAAACTAAAGAAAATGAGATTTATCCTGTTTGGT |
| | | CCGGCCTCCCAAGTCTTCAGATGGCGGATGAGGAAT |
| | | CGCGTCTTTCCGCATACTATAATCTGCTTCATTGCTT |
| | | GCGCCGCGATTCACATAAGATTGACAACTATCTTAA |
| | | ACTTCTTAAATGTCGGATTATCCACAACAACAACTGC |
| | | TAA |
| 114 | SA20- | ATGCAACGCTTAATTGAGGATATCTGCCTCCCTCGGT |
| | A(EAAAK) | GGGGTTGCCTGTGGGAAGACGACTTTGCAGAAGCAG |
| | 3A-G129R- | CGGCGAAAGAAGCTGCCGCAAAAGAGGCCGCCGCC |
| | hPRL | AAGGCCCTCCGTGACTTATTCGACCGTGCAGTAGTGT |
| | (15-199) | TGAGCCACTATATCCATAACTTGTCTTCGGAAATGTT |
| | | CTCAGAGTTTGACAAGCGTTACACCCATGGGCGCGG |
| | | TTTTATCACGAAGGCAATTAACTCGTGCCACACATCT |
| | | AGTTTAGCCACTCCTGAGGATAAAGAACAAGCACAA |
| | | CAAATGAACCAAAAAGACTTCTTAAGTCTCATCGTC |
| | | TCGATCCTTCGCTCATGGAACGAACCTCTCTATCATC |
| | | TGGTGACGGAGGTTCGCGGTATGCAGGAAGCTCCAG |
| | | AAGCAATTCTGTCAAAAGCGGTCGAGATCGAAGAAC |
| | | AAACCAAACGGTTGTTAGAACGCATGGAGCTTATCG |
| | | TCTCGCAGGTGCATCCAGAAACAAAGGAAAATGAAA |
| | | TTTATCCGGTTTGGTCTGGTTTGCCTTCACTCCAGAT |
| | | GGCTGACGAGGAATCCCGTTTATCCGCCTACTACAA |
| | | TCTTCTCCATTGCCTCCGTCGTGATTCCCATAAAATT |
| | | GACAACTATTTGAAATTGCTCAAGTGCCGCATTATCC |
| | | ACAACAATAACTGTTGA |
| 115 | SA20-A | ATGCAACGCTTAATTGAGGATATCTGCCTCCCTCGGT |
| | (EAAAK)4 | GGGGTTGCCTGTGGGAAGACGACTTTGCAGAAGCCG |
| | A-G129R- | CCGCTAAGGAAGCAGCGGCGAAAGAAGCTGCCGCA |
| | hPRL | AAAGAGGCCGCCGCCAAGGCCCTCCGTGACTTATTC |
| | (15-199) | GACCGTGCAGTAGTGTTGAGCCACTATATCCATAAC |
| | | TTGTCTTCGGAAATGTTCTCAGAGTTTGACAAGCGTT |
| | | ACACCCATGGGCGCGGTTTTATCACGAAGGCAATTA |
| | | ACTCGTGCCACACATCTAGTTTAGCCACTCCTGAGGA |
| | | TAAAGAACAAGCACAACAAATGAACCAAAAAGACT |
| | | TCTTAAGTCTCATCGTCTCGATCCTTCGCTCATGGAA |
| | | CGAACCTCTCTATCATCTGGTGACGGAGGTTCGCGGT |
| | | ATGCAGGAAGCTCCAGAAGCAATTCTGTCAAAAGCG |
| | | GTCGAGATCGAAGAACAAACCAAACGGTTGTTAGAA |
| | | CGCATGGAGCTTATCGTCTCGCAGGTGCATCCAGAA |
| | | ACAAAGGAAAATGAAATTTATCCGGTTTGGTCTGGT |
| | | TTGCCTTCACTCCAGATGGCTGACGAGGAATCCCGTT |
| | | TATCCGCCTACTACAATCTTCTCCATTGCCTCCGTCG |
| | | TGATTCCCATAAAATTGACAACTATTTGAAATTGCTC |
| | | AAGTGCCGCATTATCCACAACAATAACTGTTGA |
| 116 | SA20- | ATGCAGCGCCTTATTGAGGACATTTGTTTACCGCGTT |
| | A(EAAAK) | GGGGTTGCCTGTGGGAAGACGACTTCGCAGAAGCTG |
| | 5A-G129R- | CTGCCAAAGAGGCGGCAGCTAAGGAGGCCGCCGCTA |
| | hPRL | AAGAAGCAGCAGCTAAAGAGGCAGCTGCAAAAGCC |
| | (15-199) | TTGCGCGATTTATTTGATCGCGCCGTGGTCCTGTCCC |
| | | ACTATATTCATAACTTATCATCAGAAATGTTCAGTGA |
| | | GTTCGATAAACGGTACACACATGGGCGGGGCTTCAT |
| | | TACGAAGGCCATTAACTCATGTCATACATCAAGTCT |
| | | GGCAACACCAGAGGATAAAGAACAGGCTCAACAGA |
| | | TGAACCAGAAAGACTTCCTTTCATTGATTGTCAGTAT |
| | | CTTACGGTCGTGGAATGAACCTCTGTACCATTTGGTC |
| | | ACTGAAGTCCGTGGGATGCAGGAGGCCCCGGAGGCA |
| | | ATTTTGTCGAAAGCGGTGGAGATCGAAGAACAGACT |
| | | AAACGGTTGCTCGAACGCATGGAACTGATCGTATCT |
| | | CAGGTTCATCCTGAAACTAAGGAAAATGAGATCTAC |
| | | CCTGTCTGGAGTGGGTTACCGTCGCTTCAAATGGCG |
| | | GATGAGGAGTCGCGTTTGTCTGCATATTACAACCTCC |
| | | TTCACTGTCTCCGGCGCGACTCTCACAAAATCGATAA |
| | | TTATCTCAAGTTACTCAAGTGCCGCATTATTCACAAT |
| | | AATAACTGTTAA |
| 117 | SA20-[A | ATGCAGCGCCTGATCGAGGATATCTGTCTTCCTCGTT |
| | (EAAAK) | GGGGGTGTCTGTGGGAGGACGACTTTGCGGAAGCAG |
| | 4A]2- | CGGCAAAGGAAGCCGCTGCCAAAGAAGCCGCAGCG |
| | G129R- | AAAGAGGCCGCCGCGAAAGCTGCCGAGGCCGCTGCC |
| | hPRL | AAGGAGGCTGCGGCGAAGGAAGCTGCTGCCAAGGA |
| | (15-199) | AGCGGCGGCGAAAGCATTACGTGATTTGTTTGATCG |
| | | GGCAGTTGTCCTGAGCCATTACATCCACAATTTGTCC |
| | | TCAGAAATGTTTTCGGAGTTTGACAAGCGCTATACCC |
| | | ATGGGCGGGGCTTCATCACCAAAGCAATCAACTCCT |
| | | GCCATACTTCTTCTCTTGCCACACCTGAAGATAAAGA |
| | | ACAAGCTCAACAGATGAATCAGAAAGACTTTTTGTC |
| | | GCTCATTGTGAGTATTCTGCGGTCTTGGAATGAACCG |
| | | CTCTACCACCTTGTTACGGAGGTCCGTGGGATGCAA |
| | | GAAGCGCCGGAGGCGATTCTCAGCAAAGCCGTGGAG |
| | | ATTGAAGAGCAGACGAAGCGCCTCCTTGAGCGTATG |
| | | GAACTCATTGTGAGTCAAGTACACCCGGAAACAAAG |
| | | GAGAATGAAATCTACCCTGTCTGGTCAGGCTTACCTT |
| | | CTTTACAGATGGCAGATGAAGAGTCACGTCTCTCTG |
| | | CCTATTACAACTTACTGCATTGCTTACGGCGTGATAG |
| | | TCATAAAATCGACAATTACCTTAAACTGTTAAAGTGT |
| | | CGCATTATCCATAATAATAATTGCTGA |
| 118 | SA20-(A | ATGCAACGCTTAATCGAAGATATTTGTCTCCCGCGCT |
| | (EAAAK)4 | GGGGGTGCCTGTGGGAGGACGATTTTGCTGAGGCTG |
| | ALEA(EAAA | CAGCGAAGGAGGCTGCGGCTAAAGAGGCAGCCGCA |
| | K)4A)- | AAAGAAGCAGCGGCAAAAGCTTTAGAGGCAGAGGC |
| | G129R- | TGCCGCGAAGGAAGCCGCAGCAAAAGAGGCGGCGG |
| | hPRL | CCAAGGAGGCAGCCGCTAAGGCCCTGCGTGACCTTT |
| | (15-199) | TCGATCGTGCAGTAGTGTTATCGCACTATATCCATAA |
| | | CTTGTCCTCCGAAATGTTTAGCGAATTTGATAAACGG |
| | | TACACGCACGGGCGCGGGTTTATCACGAAAGCGATT |
| | | AACTCCTGCCATACAAGCAGTCTGGCCACACCGGAA |
| | | GATAAGGAACAGGCACAACAAATGAACCAAAAGGA |
| | | CTTCTTGTCTCTGATTGTTTCGATTTTACGGTCTTGG |
| | | AACGAGCCGCTTTACCATCTCGTAACCGAAGTCCGCG |
| | | GGATGCAGGAGGCCCCAGAAGCGATCTTAAGTAAGG |
| | | CAGTTGAGATTGAGGAACAAACTAAACGGTTACTCG |
| | | AACGGATGGAGCTGATCGTCAGTCAAGTTCATCCAG |
| | | AAACTAAGGAAAACGAGATTTATCCAGTATGGAGTG |
| | | GGTTACCAAGCCTCCAAATGGCTGATGAGGAGAGTC |
| | | GTTTATCAGCTTACTACAACTTGCTTCATTGCCTGCG |
| | | CCGTGACTCACACAAAATTGATAATTACCTTAAGCTC |
| | | CTCAAGTGTCGTATCATCCATAATAATAACTGCTGA |
| 119 | SA20- | ATGCAGCGTCTGATTGAGGACATTTGCCTTCCACGCT |
| | (EAAAK)₃- | GGGGCTGTTTATGGGAGGACGATTTTGAGGCTGCCG |
| | G120R- | CCAAGGAGGCCGCAGCCAAAGAGGCTGCCGCTAAG |
| | hPL | CTGTCCCGCCTCTTTGACCACGCAATGCTCCAAGCTC |
| | (6-191) | ACCGGGCTCATCAATTGGCGATCGACACGTACCAGG |
| | | AATTTGAAGAAACATACATTCCGAAAGATCAGAAAT |
| | | ATAGTTTTCTGCATGACTCGCAGACTTCGTTTTGCTT |
| | | TAGCGACAGTATCCCAACGCCATCGAACATGGAGGA |
| | | GACGCAACAAAAGTCAAACTTAGAGTTACTGCGTAT |
| | | TAGCCTCTTGTTGATTGAGTCCTGGCTCGAACCAGTA |
| | | CGGTTTTTACGGAGTATGTTTGCAAATAATCTCGTCT |
| | | ACGACACTAGTGATAGTGACGACTATCATTTACTCA |
| | | AGGATCTTGAAGAGCGGATTCAGACCCTCATGGGTC |
| | | GTCTTGAAGATGGGTCCCGCCGGACCGGTCAAATCC |
| | | TGAAACAAACCTACTCCAAGTTCGACACAAACAGCC |
| | | ATAATCACGACGCCTTATTAAAAAACTATGGCTTACT |
| | | GTATTGCTTCCGCAAGGACATGGACAAAGTGGAGAC |
| | | ATTCTTGCGTATGGTGCAATGTCGCTCGGTGGAGGG |
| | | CAGTTGCGGTTTTTGA |
| 120 | SA20- | ATGCAACGCCTGATCGAAGACATCTGTTTGCCACGTT |
| | (EAAAK)₃- | GGGGCTGCCTCTGGGAGGATGACTTCGAGGCTGCCG |
| | G120R- | CCAAGGAGGCCGCAGCCAAAGAGGCTGCCGCTAAG |
| | hGH | CTTAGTCGTCTTTTTGATAATGCAATGCTCCGTGCGC |
| | (6-191) | ATCGCCTCCATCAGCTCGCTTTTGACACCTACCAGGA |
| | | GTTCGAGGAAGCGTACATTCCTAAAGAACAGAAATA |
| | | CTCGTTCCTCCAGAACCCACAGACCTCGTTATGTTTC |
| | | AGTGAAAGCATTCCAACCCCATCCAATCGGGAAGAG |
| | | ACACAGCAAAAGTCTAATTTGGAGTTGCTCCGCATTT |
| | | CGCTTCTCCTTATTCAGTCTTGGCTCGAACCTGTTCA |
| | | GTTCCTCCGGTCCGTCTTTGCCAATTCCTTAGTCTAC |
| | | GGGGCATCGGATAGCAATGTCTATGACCTTTTGAAA |
| | | GACTTGGAAGAACGCATTCAGACTTTGATGGGGCGT |
| | | TTGGAAGACGGTTCACCTCGTACGGGCCAGATTTTC |
| | | AAACAAACTTATAGCAAATTTGACACGAACTCACAT |
| | | AATGATGACGCATTGCTCAAAAACTACGGGCTGCTT |
| | | TACTGCTTTCGGAAGGACATGGATAAGGTGGAGACT |
| | | TTTCTGCGGATTGTACAGTGTCGGAGCGTCGAAGGG |
| | | AGCTGTGGCTTCTAA |

### Production of Fusion Polypeptides

The present disclosure provides, among other things, methods of producing polypeptides (e.g., fusion polypeptides) described herein. In one aspect, the present disclosure provides methods of producing fusion polypeptides as described herein, comprising contacting a cell with a nucleic acid encoding the fusion polypeptide, wherein contacting occurs under conditions sufficient to permit (i) uptake of the nucleic acid by the cell and (ii) translation of the fusion polypeptide.

Such conditions sufficient to permit (i) uptake of the nucleic acid by the cell and (ii) translation of the fusion polypeptide are well known in the art and the skilled artisan would readily be able to select and use a plurality of methods and/or conditions that could be successfully utilized. For example, a variety of methods are readily available and known to those of ordinary skill in the art to introduce a nucleic acid (e.g., a vector) into host cells. This can include, without limitation, introduction of a nucleic acid into a host cell using transfection (e.g., by lipofection, calcium phosphate transfection) or transduction. Following introduction of a nucleic acid into host cells, such cells can be cultured for a period of time to allow for translation of the fusion polypeptide. Cell culture conditions which permit translation of polypeptides (e.g., fusion polypeptides) are readily understood. The choice of such conditions (e.g., to permit uptake of the nucleic acid by the cell and translation of the fusion polypeptide) can depend on a number of factors, such as cell type and the particular parameters of the nucleic acid to be contacted (e.g., DNA vs. RNA). Selection of such techniques and/or culture conditions are well within the level of the skilled artisan.

For example, host cells may be Escherichia coli (E. coli) cells. E. coli can be used to quickly and economically produce large quantities of recombinant proteins (e.g., fusion polypeptides described herein); however, over-expression of recombinant proteins in E. coli can have a toxic effect. One way to minimize the toxic effects is, for example, to clone the gene into a vector with a controllable promoter. A controllable expression system can allow the culture to grow to high density before inducing expression of the recombinant protein.

Recombinant expression of fusion polypeptides (e.g., in E. coli) can result in the accumulation of fusion polypeptides in inclusion bodies. In some embodiments, methods of producing polypeptides (e.g., fusion polypeptides) described herein comprise purifying or isolating inclusion bodies. Such methods of purifying or isolating inclusion bodies can comprise, for example, cell lysis (e.g., by mechanical methods, by chemical methods). Due, at least in part, to the high density of inclusion bodies compared to other cellular components, inclusion bodies can be purified or isolated from whole cell lysates by centrifugation or cross-flow membrane microfiltration.

In some embodiments, inclusion bodies can be subsequently solubilized. Solubilization can be completed by, for example, using high concentration denaturants and/or chaotropes, such as urea and guanidine hydrochloride. In some embodiments, β-mercaptoethanol or dithiothreitol are alternatively or additionally utilized for solubilization. Such denaturants and/or chaotropes are typically utilized in a solution (e.g., a buffered solution, such as Tris, phosphate, or HEPES). Solubilized material can be clarified by centrifugation, decanted, and/or retained. Solubilization of inclusion bodies using high concentration of chaotropes can result in disruption and/or unfolding of fusion polypeptide structure, and, in some cases, can lead to aggregation of fusion polypeptide molecules. Non-covalent aggregation of fusion polypeptides indicative of structural disruption or unfolding can be evaluated by a plurality of methods, including, for example, Native Polyacrylamide Gel Electrophoresis (Native PAGE). Native PAGE separates polypeptides by size and charge and can provide insight to the composition and structure of the purified or isolated fusion polypeptides. Without wishing to be bound by any one theory, it is understood that the migration of polypeptides through the gel is affected by both the charge and the shape of the polypeptide. Separated polypeptides can be stained with a dye or stain and imaged with a gel imaging system. Profiles can be compared to each other and polypeptide molecular weight standards to determine if there is non-covalent aggregation of fusion polypeptides indicative of misfolding.

In some embodiments, solubilized (e.g., disrupted and/or unfolded) fusion polypeptides are refolded. A plurality of methods can be utilized to refold such fusion polypeptides. For example, solubilized fusion polypeptides can be refolded by dilution. Refolding by dilution can comprise adding a fusion polypeptide to a refolding solution, either rapidly (e.g., over the course of 1-30 minutes) or slowly (overly the course of 30 minutes to 5 hours) to a certain concentration of fusion polypeptide, such as 0.5 mg/mL to 3.0 mg/mL. In some embodiments, the refolding solution is maintained between about 7.3 and about 8.5. In some embodiments, a refolding solution comprises L-arginine (e.g., to prevent aggregation). In some embodiments, a refolding solution comprises a mixture of reduced and oxidized molecules at a certain ratio (e.g., to initiate disulfide bonding), for example about a 1:1 to about a 6:1 ratio. Such mixtures of reduced and oxidized molecules may comprise cysteine and cystine or reduced glutathione and oxidized glutathione. Refolding by dilution can performed in a vessel open to the atmosphere or sparged with air or nitrogen (e.g., at a temperature between 4-30° C.).

Solubilized fusion polypeptides can be refolded by chromatographic methods, such as gradient size exclusion chromatography. Use of size exclusion chromatography for refolding can result in separation of the folded form from the aggregated and misfolded forms during elution with the refolding buffer. In other chromatographic methods, refolding process can occur after immobilization of the polypeptide on the solid support that leads to spatial separation of the refolding units and decreased intermolecular interactions. Thus, refolding processes in chromatographic beds can be carried out at high protein concentrations.

Solubilized fusion polypeptides can be refolded by dialysis. In dialysis, a disrupted and/or unfolded fusion polypeptide is present in a buffer or solution comprising a denaturant and, by sufficiently decreasing the denaturant concentration via the dialaysis, refolding is facilitated.

In some embodiments, methods of producing fusion polypeptides of the present disclosure further comprises isolating or purifying the fusion polypeptides from undesired sample components (e.g., culture media, host cell contaminants, other polypeptides or nucleic acids). A plurality of polypeptide isolation/purification methods are known in the art. One of ordinary skill, reading the present disclosure, would readily recognize and understand how to select and use such methods in accordance with technologies of the present disclosure. For example, and without limitation, polypeptides (e.g., fusion polypeptides described herein) can be isolated or purified by chromatographic methods (e.g., high-performance liquid chromatography, size exclusion chromatography, ion exchange chromatography, affinity chromatography) or other affinity-based methods (e.g., purification utilizing an epitope tag). See, e.g., Coskun O. et al. North Clin Istanb. 2016 Nov. 11; 3 (2): 156-160, Mishra V. et al. Curr Protein Pept Sci. 2020; 21 (8): 821-830. Fusion polypeptides may also be isolated or purified according to their isoelectric point, for example, by isoelectric focusing.

In some embodiments, methods of producing fusion polypeptides of the present disclosure further comprise desalting and buffer exchange (e.g., to remove salts and/or low molecular weight contaminants). Desalting and buffer exchange can be completed, for example, by buffer exchange gel filtration chromatography. In some embodiments, purified or isolated fusion polypeptides undergo a final filtration.

### Characterization

In one aspect, the present disclosure provides methods of characterizing fusion polypeptides, nucleic acids, and/or compositions described herein. Characterization can be performed during and/or following a production process. In some embodiments, a particular production process may be modified and/or terminated in light of a characterization (e.g., if a particular preparation of a fusion polypeptide or composition comprising the same fails to meet one or more specifications). Characterization can include assessing one or more of: (i) signaling activity of the fusion polypeptide; (ii) affinity of the fusion polypeptide to serum albumin; (iii) affinity of the fusion polypeptide to its receptor; (iv) purity of the fusion polypeptide; (v) yield of the fusion polypeptide; (vi) efficacy of treating a subject having a cancer; and (vii) half-life of the fusion polypeptide. Fusion polypeptides, nucleic acids, or compositions of the present disclosure may alternatively or additionally be characterized for their pharmacokinetic and/or pharmacodynamic properties.

In some embodiments, signaling activity of fusion polypeptides as described herein are characterized for signaling activity. A plurality of methods are available and known to those of ordinary skill in the art to assess signaling activity (e.g., compared to a reference). Such methods include, for example, in vitro- or in vivo-based signaling activity assays.

In some embodiments, signaling activity is assessed by an in vitro signaling activity assay. Such an assay can comprise, for example, measuring inhibition (e.g., antagonism) of downstream signaling from the receptor of the polypeptide hormone analog of the fusion polypeptide. Measuring inhibition (e.g., antagonism) of downstream signaling from the receptor of the polypeptide hormone analog of the fusion polypeptide can utilize a reporter (also referred to as a “reporter assay”). Reporter assays can measure activity (or a reduction thereof) using a detectable molecule (e.g., a “reporter”) that correlates with fusion polypeptide activity.

In some embodiments, signaling activity is assessed by an in vivo signaling activity assay. Such an assay can comprise, for example, administering a fusion polypeptide (or composition comprising such a fusion polypeptide or nucleic acid encoding the same) to a subject (e.g., a mouse) and evaluating signaling activity. Signaling activity can be evaluated, for example, by measuring inhibition (e.g., antagonism) of downstream signaling from the receptor of the polypeptide hormone analog of the fusion polypeptide (e.g., compared to an appropriate reference). A variety of methods are available to measure inhibition (e.g., antagonism) of downstream signaling of a fusion polypeptide described herein. For example, differential gene expression, protein expression, and/or alterations in post-translational modifications induced by a fusion polypeptide can be measured.

For example, inhibition (e.g., antagonism) of the prolactin or growth hormone receptors can be assessed, for example, by measuring dose-dependent inhibition of receptor-induced STAT5 phosphorylation, AKT phosphorylation and/or ERK1/2 phosphorylation (e.g., relative to a reference).

In some embodiments, fusion polypeptides as described herein are characterized for their affinity to serum albumin and/or the receptor of the polypeptide hormone analog of the fusion polypeptide. A plurality of techniques to assess affinity are known in the art and their use is well within the level of one of ordinary skill in the art. Examples of such techniques include, without limitation, fluorescence resonance energy transfer (FRET), enzyme-linked immunosorbent assays (ELISA), surface plasmon resonance (e.g., “Biacore”), and bioluminescent resonance energy transfer (BRET).

In some embodiments, fusion polypeptides and/or nucleic acids encoding a fusion polypeptide as described herein are characterized by assessing their purity. Purity of a fusion polypeptide and/or nucleic acid encoding a fusion polypeptide can be determined by a plurality of methods known in the art and readily understood by those of ordinary skill, such as analytical chemistry techniques, for example, polyacrylamide gel electrophoresis (e.g., Sodium dodecyl-sulfate polyacrylamide gel electrophoresis (SDS-PAGE)). By utilizing such methods, a “purified” nucleic acid or fusion polypeptide may give rise to essentially one band in an electrophoretic gel.

In some embodiments, fusion polypeptides (e.g., purified fusion polypeptides) described herein are characterized by assessing their yield (or concentration). A plurality of methods are known in the art for determining polypeptide concentration and calculating yield and their use is well within the level of one of ordinary skill in their art. For example, ultraviolet (UV) absorption, biuret methods (e.g., Lowry assay, bicinchoninic acid (BCA)) assay), colorimetric methods (e.g., Bradford assay), and fluorescent dye methods can be utilized to determine polypeptide concentration. Certain analytical techniques may also be utilized, such as chromatography. In some embodiments, polypeptide concentration (e.g., fusion polypeptide concentration) is determined utilizing UV-light absorbance (A280) with an extinction coefficient (ϵ280). Such a method can be particularly useful when a composition comprising fusion polypeptides is highly concentrated (e.g., outside the range of a Bradford assay).

In some embodiments, fusion polypeptides (e.g., compositions comprising fusion polypeptides) described herein are characterized by assessing their efficacy of treating a subject having a cancer. Efficacy can be characterized according to a variety of methods available and known in the art. For example, a fusion polypeptide as described herein can be administered (e.g., by intravenous injection) to a subject and efficacy is determined in comparison to an appropriate reference. Efficacy can include, for example, an inhibition of tumor growth, a decrease in tumor growth, a decrease in tumor volume, prevention and/or reduction of metastasis, prevention of recurrence, and/or increased survival (e.g., duration of survival). Efficacy can be determined pre-clinically (e.g., in an animal model) or clinically (e.g., in a human subject). In some embodiments, fusion polypeptides, nucleic acids, or compositions described herein can inhibit tumor growth, decrease tumor growth, decrease tumor volume, prevent and/or reduce metastasis, prevent recurrence, and/or increase survival relative to a reference (e.g., placebo).

In some embodiments, the half-life of fusion polypeptides described herein is assessed. Half-life (also referred to as “elimination half-life”) is the length of time required for the concentration of a particular substance (e.g., a fusion polypeptide as described herein) to decrease to half of its starting dose in the body. A variety of methods are known in the art to evaluate half-life. For example, following administration of a fusion polypeptide (or a composition comprising a fusion polypeptide) to a subject, a blood sample (or a plurality blood samples, e.g., over a period of time) can be obtained. Subsequently, the blood sample can be assessed for the amount of fusion polypeptide present is measured, for example, by enzyme-linked immunosorbent assays (ELISAs), radioimmunoassays (RIAs), or immunoradiometric assays (IRMAs) if protein-specific antibodies are available; otherwise, high-performance liquid chromatography or mass spectrometry. In some embodiments, fusion polypeptides described herein are characterized to have an increased half-life (e.g., in vivo half-life) relative to a reference (e.g., a polypeptide hormone analog not part of a fusion polypeptide as described herein).

In some embodiments, endotoxin levels in compositions of the present disclosure are characterized. In some embodiments, endotoxin levels are determined as less than 1 EU/mL. In some embodiments, endotoxin levels are determined as greater than 1 EU/mL. In instances wherein endotoxin levels are determined as greater than 1 EU/mL, such preparations of fusion polypeptides or nucleic acids and/or compositions may be discarded and a new composition may be produced (e.g., with a determined endotoxin level less than 1 EU/mL).

In some embodiments, fusion polypeptides or nucleic acids and/or compositions of the present disclosure are characterized for their in vivo pharmacokinetic and/or pharmacodynamic parameters. A plurality of methods are known in the art and readily available which would be suitable to evaluate the pharmacokinetic and/or pharmacodynamic parameters of technologies of the present disclosure. It is well within the level of one of ordinary skill in the art to select and use such methods to evaluate technologies described herein.

### Compositions

The present disclosure also provides compositions comprising a fusion polypeptide of the present disclosure, a nucleic acid encoding a fusion polypeptide of the present disclosure, a fusion polypeptide and a second therapeutic agent of the present disclosure, or a host cell comprising a fusion polypeptide and/or a nucleic acid encoding a fusion polypeptide of the disclosure. Such compositions can be a pharmaceutical composition. Pharmaceutical compositions can also comprise one or more pharmaceutically acceptable carriers.

A pharmaceutically acceptable carrier is a pharmaceutically-acceptable material, composition or vehicle, such as a liquid or solid filler, diluent, excipient, or solvent encapsulating material, involved in carrying and/or transporting the active agent (e.g., fusion polypeptide, second therapeutic agent) from one organ, or portion of the body, to another organ, or portion of the body. Each carrier must be “acceptable” in the sense of being compatible with the other ingredients of the formulation and not injurious to the patient. Appropriate pharmaceutically acceptable carriers, including but not limited to excipients and stabilizers, are known in the art. For example, a pharmaceutically acceptable carrier can be sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or a buffer, an emulsifying agent, a dispersing agent, an isotonic agent, a wetting agent, a chelating agent, a sequestering agent, a pH buffering agent, a solubility enhancer, an antimicrobial agent, and/or an antioxidant. See, e.g., Remington's Pharmaceutical Sciences (1990) Mack Publishing Co., Easton, PA; Remington: The Science and Practice of Pharmacy, 21st Edition, A. R. Gennaro, Lippincott, Williams & Wilkins, Baltimore, MD, 2006.

Pharmaceutical compositions of the present disclosure can comprise one or more pharmaceutically acceptable salts. Such salts are well known in the art. See, e.g., S. M. Berge, et al. J. Pharmaceutical Sciences, 66:1-19 (1977). Examples of pharmaceutically acceptable salts that can be included in pharmaceutical compositions of the present disclosure include, without limitation, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

Pharmaceutical compositions can be formulated such that they are suitable for administration to a subject (e.g., a human). For example, pharmaceutical compositions can be formulated such that the pharmaceutical composition comprises a therapeutically effective amount of a fusion polypeptide of the present disclosure, a nucleic acid encoding a fusion polypeptide of the present disclosure, a fusion polypeptide of the present disclosure and a second therapeutic agent, or a host cell comprising a fusion polypeptide a nucleic acid encoding a fusion polypeptide of the disclosure.

A pharmaceutical composition may be formulated for any route of administration. In some embodiments, a pharmaceutical composition is formulated for subcutaneous injection (e.g., an injectable pharmaceutical composition). In some embodiments, a pharmaceutical composition is formulated for intravenous administration. In some embodiments, a pharmaceutical composition is a sustained release or long-lasting pharmaceutical composition (e.g., comprises an increased half-life relative to an appropriate reference).

### Uses

Technologies of the present disclosure are useful in a plurality of methods, including, for example, methods of treating a disease and/or disorder in a subject. In one aspect, the present disclosure provides a method of treating a disease and/or disorder in a subject comprising administering a therapeutically effective amount of a fusion polypeptide of the present disclosure, a nucleic acid encoding a fusion polypeptide of the present disclosure, or a composition described herein to the subject.

In some embodiments, fusion polypeptides, nucleic acids encoding fusion polypeptides, or certain compositions of the present disclosure are administered as a monotherapy (e.g., in a therapeutically effective amount).

In some embodiments, fusion polypeptides, nucleic acids encoding fusion polypeptides, and compositions of the present disclosure are administered in combination with a therapeutically effective amount of a second therapeutic agent (e.g., as a “combination therapy”). A second therapeutic agent can be any anti-cancer agent known in the art that may be suitable for use in combination with technologies of the present disclosure. For example, a second therapeutic agent may be a chemotherapy or a radiotherapy. In some embodiments, the second therapeutic agent is a chemotherapy. In some such embodiments, the chemotherapy is selected from the group consisting of: carboplatin, cisplatin, docetaxel, and paclitaxel.

In some embodiments, the second therapeutic agent is an agent for treatment of gynecological cancer. Agents for the treatment of gynecological cancer include, but are not limited to, therapies involving antibodies, small molecules (e.g., chemotherapeutics), hormones (e.g., steroidal, polypeptide, and the like), radiotherapies (e.g., γ-rays, X-rays, and/or the directed delivery of radioisotopes, microwaves, UV radiation and the like), gene therapies (e.g., antisense, retroviral therapy and the like) and other immunotherapies. In some embodiments, fusion polypeptides, nucleic acids encoding fusion polypeptides, and compositions of the present disclosure are administered in combination with a taxane (e.g., paclitaxel) or platinum-based chemotherapy (e.g., carboplatin).

In some embodiments, the subject is a mammalian subject (e.g., a mouse, a rat, a horse, a dog, a non-human primate, a human). In some embodiments, the subject is a human subject. In some embodiments, the human subject is a female human subject. In some embodiments, the subject is a subject that has cancer.

In some embodiments, the disease and/or disorder is cancer. Cancer is a disease that can result in one or more of: unregulated cell division (or increased survival or increased resistance to cell death), penetration of the cell into another neighboring tissue (invasion); and proliferation through the blood vessels (metastasis). Cancers of the present disclosure can be a resistant cancer (e.g., chemotherapy-resistant cancer, platinum-resistant cancer), a refractory cancer (e.g., a chemotherapy-refractory cancer, platinum-refractory cancer), a recurrent cancer, and/or a metastatic cancer. In some embodiments, a cancer may involve one or more tumors.

Tests for diagnosing the cancers to be treated by the methods described herein are known in the art and will be familiar to the ordinary medical practitioner. These laboratory tests include, without limitation, microscopic analyses, cultivation dependent tests (such as cultures), and nucleic acid detection tests. These include wet mounts, stain-enhanced microscopy, immune microscopy (e.g., FISH), hybridization microscopy, particle agglutination, enzyme-linked immunosorbent assays, urine screening tests, DNA probe hybridization, serologic tests, etc. The medical practitioner generally takes a full history and conducts a complete physical examination in addition to running the laboratory tests listed above.

In some embodiments, the cancer is a resistant cancer. In some embodiments, the resistant cancer is a chemotherapy-resistant cancer. In some embodiments, the resistant cancer is a platinum-resistant cancer.

In some embodiments, the cancer is a refractory cancer. In some embodiments, the refractory cancer is a chemotherapy-refractory cancer. In some embodiments, the refractory cancer is a platinum-refractory cancer.

In some embodiments, the cancer is a breast cancer, a gynecological cancer, prostate cancer, or melanoma. Breast cancers can include, for example, luminal breast cancer, basal-like breast cancer, normal-like breast cancer, ductal carcinoma in situ, invasive breast cancer, triple-negative breast cancer, human epidermal growth factor receptor 2 (HER2) positive breast cancer, inflammatory breast cancer, Paget disease of the breast, angiosarcoma, hormone receptor positive breast cancer (e.g., Estrogen Receptor and/or Progastrin Receptor positive), and phyllodes tumor. Gynecological cancers include, for example, ovarian cancer, peritoneal cancer, cervical cancer, uterine cancers (e.g., endometrial cancer, uterine sarcoma), vaginal cancer, vulvar cancer, and fallopian tube cancer. Prostate cancers include, for example, adenocarcinoma of the prostate, transitional cell carcinoma of the prostate, squamous cell carcinoma of the prostate, and small cell prostate cancer. Melanomas include, for example, superficial spreading melanoma, nodular melanoma, lentigo maligna melanoma, and acral lentiginous melanoma.

In some embodiments, the breast cancer is a luminal breast cancer. In some embodiments, the breast cancer is a basal-like breast cancer. In some embodiments, the breast cancer is a normal-like breast cancer. In some embodiments, the breast cancer is a ductal carcinoma in situ. In some embodiments, the breast cancer is an invasive breast cancer, triple-negative breast cancer. In some embodiments, the breast cancer is a human epidermal growth factor receptor 2 (HER2) positive breast cancer. In some embodiments, the breast cancer is an inflammatory breast cancer. In some embodiments, the breast cancer is a Paget disease of the breast. In some embodiments, the breast cancer is an angiosarcoma. In some embodiments, the breast cancer is a hormone receptor positive breast cancer (e.g., Estrogen Receptor and/or Progastrin Receptor positive). In some embodiments, the breast cancer is a phyllodes tumor.

In some embodiments, the gynecological cancer is an ovarian cancer. In some embodiments, the ovarian cancer is an epithelial ovarian carcinoma. In some embodiments, the ovarian cancer is a germ cell tumor. In some embodiments, the ovarian cancer is a stromal cell tumor. In some embodiments, the ovarian cancer is a platinum-resistant ovarian cancer.

In some embodiments, the platinum-resistant ovarian cancer is a folate-receptor alpha positive (FRα+) platinum-resistant ovarian cancer.

In some embodiments, the gynecological cancer is a peritoneal cancer. In some embodiments, the gynecological cancer is a cervical cancer. In some embodiments, the gynecological cancer is a uterine cancer. In some embodiments, the uterine cancer is uterine sarcoma. In some embodiments, the uterine cancer is an endometrial cancer. In some embodiments, the gynecological cancer is a vaginal cancer. In some embodiments, the cancer is a vulvar cancer. In some embodiments, the gynecological cancer is a fallopian tube cancer.

In some embodiments, the prostate cancer is an adenocarcinoma of the prostate. In some embodiments, the prostate cancer is transitional cell carcinoma of the prostate. In some embodiments, the prostate cancer is squamous cell carcinoma of the prostate. In some embodiments, the prostate cancer is small cell prostate cancer.

In some embodiments, the cancer is a melanoma. In some embodiments, the melanoma is superficial spreading melanoma. In some embodiments, the melanoma is nodular melanoma. In some embodiments, the melanoma is lentigo maligna melanoma. In some embodiments, the melanoma is acral lentiginous melanoma.

In some embodiments, the disease and/or disorder is an autoimmune or inflammatory disease and/or disorder (e.g., when the receptor of a fusion polypeptide of the present disclosure is prolactin receptor). Such diseases and/or disorders can include, for example, rheumatoid arthritis, lupus (e.g., systemic lupus erythematosus), and Reiter's syndrome.

The increased in vivo half-life of fusion polypeptides of the present disclosure can facilitate a decrease in the frequency of administration of fusion polypeptides, nucleic acids encoding fusion polypeptides, and compositions of the present disclosure, even by subcutaneous injection, relative to a reference (e.g., currently approved polypeptide hormone antagonists, such as Pegvisomant). In some embodiments, fusion polypeptides, nucleic acids encoding fusion polypeptides, and compositions of the present disclosure are administered every other day, every three days, every four days, every five days, every six days, one per week, one per two weeks, once per three weeks, or once per month.

When a fusion polypeptides, nucleic acids encoding fusion polypeptides, and compositions of the present disclosure are administered in combination with a chemotherapy, the increased in vivo half-life of fusion polypeptides of the present disclosure can facilitate one-per-chemotherapy cycle administration of a fusion polypeptides, nucleic acids encoding fusion polypeptides, and compositions of the present disclosure.

A therapeutically effect amount of a fusion polypeptide, a nucleic acid encoding a fusion polypeptide, a second therapeutic agent, or a composition of the present disclosure can be administered to a subject by any route of introducing or delivering the agent or composition to a subject to perform its intended function. Administration can be carried out by any suitable route, including, but not limited to, intravenously, intramuscularly, intraperitoneally, and/or subcutaneously. Administration can include self-administration and administration by another.

To treat a disease and/or disorder (e.g., cancer) in an animal model or subject, a fusion polypeptide, a nucleic acid encoding a fusion polypeptide, a second therapeutic agent, or a composition of the present disclosure may be delivered systemically (e.g., by intravenous administration into the bloodstream) or by local administration (e.g., by intratumoral or intramuscular injection). Alternatively or additionally, a fusion polypeptide, a nucleic acid encoding a fusion polypeptide, a second therapeutic agent, or a composition of the present disclosure can be administered to the vasculature of a tumor (e.g., a malignant tumor). Fusion polypeptides of the present disclosure are particularly suitable for systemic administration. Thus, in a preferred embodiment, fusion polypeptides described herein are administered systemically. Preferably, administration is parenteral (e.g., intravenous or intravenous injection, infusion or implantation).

In some embodiments, fusion polypeptides, nucleic acids encoding fusion polypeptides, second therapeutic agents and/or compositions of the present disclosure are administered peripherally. In some embodiments, fusion polypeptides, nucleic acids encoding fusion polypeptides, second therapeutic agents and/or compositions of the present disclosure are administered intravenously. In some embodiments, fusion polypeptides, nucleic acids encoding fusion polypeptides, second therapeutic agents and/or compositions of the present disclosure are administered by subcutaneous injection.

In some embodiments, a therapeutically effective amount of a fusion polypeptide, a nucleic acid encoding a fusion polypeptide, or a composition is administered via a first route of administration (e.g., intravenously) and a second therapeutic agent is administered via a second route of administration (e.g., orally). In some embodiments, a therapeutically effective amount of a fusion polypeptide, a nucleic acid encoding a fusion polypeptide, or a composition and a second therapeutic agent are administrated by the same route of administration.

In some embodiments, a therapeutically effective amount of a fusion polypeptide, a nucleic acid encoding a fusion polypeptide, or a composition is administered first, followed by administration of a second therapeutic agent. In some embodiments, a therapeutically effective amount of a fusion polypeptide, a nucleic acid encoding a fusion polypeptide, or a composition is administered following administration of a second therapeutic agent. In some embodiments, a therapeutically effective amount of a fusion polypeptide, a nucleic acid encoding a fusion polypeptide, or a composition is administered at about the same time as a second therapeutic agent.

In some embodiments, fusion polypeptides, nucleic acids encoding fusion polypeptides, a second therapeutic agent, or compositions of the present disclosure are administered before, during, and/or after surgery.

As will be readily apparent to those of ordinary skill in the art, the amounts (e.g., therapeutically effective amounts) of a of fusion polypeptides, nucleic acids encoding fusion polypeptides, and compositions of the present disclosure, alone, or in combination with a second therapeutic agent (e.g., sufficient to reduce tumor growth and/or size) will vary not only on the particular agents selected, but also with the route of administration, the nature of the condition being treated, the age and/or condition of the subject being treated, and will ultimately be at the discretion of the subject's health care provider, pharmacist, and/or based upon clinical guidelines. The length of time during which the treatments of the present disclosure is administered can vary on an individual basis and/or be based upon clinical guidelines.

The response (e.g., clinical response) of the subject, disease, and/or disorder treated with technologies of the present disclosure can be monitored. A response may refer to an alteration in a subject's condition that occurs as a result of or correlates with a treatment described herein. In some embodiments, a response is or comprises a beneficial response. In some embodiments, a beneficial response may include stabilization of the disease and/or disorder (e.g., prevention or delay of deterioration expected or typically observed to occur absent the treatment), amelioration (e.g., reduction in frequency and/or intensity) of one or more symptoms of the disease and/or disorder, and/or improvement in the prospects for cure of the disease and/or disorder.

In some embodiments, presence, extent, and/or nature of response may be measured and/or characterized according to particular criteria; in some embodiments, such criteria may include clinical criteria and/or objective criteria. In some embodiments, techniques for assessing response may include, but are not limited to, clinical examination, positron emission tomography, chest X-ray CT scan, MRI, ultrasound, endoscopy, laparoscopy, presence or level of a particular marker in a sample, cytology, and/or histology. Where a response of interest is or comprises response of a cancer to therapy, those of ordinary skill will be aware of a variety of established techniques for assessing such response, including, for example, for determining tumor burden, tumor size, and/or tumor stage. For example, certain technologies for assessing response of solid tumors to treatment are discussed in Therasse et. al., J. Natl. Cancer Inst., 2000, 92 (3): 205-216. Those of ordinary skill in the art will be aware of, and/or will appreciate in light of the present disclosure, strategies for determining particular response criteria for individual tumors, tumor types, patient populations or cohorts, as well as for determining appropriate references therefore.

In some embodiments, fusion polypeptides, nucleic acids, or compositions described herein can inhibit tumor growth, decrease tumor growth, decrease tumor volume, prevent and/or reduce metastasis, prevent recurrence, and/or increase survival relative to a reference (e.g., placebo).

In some embodiments, administration of fusion polypeptides, nucleic acids encoding fusion polypeptides, and compositions of the present disclosure in combination with a second therapeutic agent synergistically inhibits tumor growth compared to a reference (e.g., compared to either a technology of the present disclosure or the second therapeutic agent when administered as a monotherapy).

### Kits

In one aspect, the present disclosure provides kits comprising fusion polypeptides, nucleic acids encoding fusion polypeptides, and/or compositions described herein. In some embodiments, a kit comprises a fusion polypeptide described herein. In some embodiments, a kit comprises a nucleic acid encoding a fusion polypeptide described herein. In some embodiments, a kit comprises a composition described herein.

In some embodiments, the kit comprises a sterile container which contains fusion polypeptides, nucleic acids encoding fusion polypeptides, or compositions described herein. In some embodiments, the sterile container further comprises a second therapeutic agent. In some embodiments, a kit comprises a second sterile container comprising a second therapeutic agent. Such sterile containers can be, for example, boxes, ampules, bottles, vials, tubes, bags, pouches, blister-packs, and/or other suitable container forms known in the art. The container may be a pre-filled syringe or auto-injection pen comprising a pharmaceutical composition described herein.

A device capable of delivering the kit components through an administrative route may be included. Examples of such devices include syringes (e.g., for parenteral administration). The device may be a pre-filled syringe or auto-injection pen comprising a pharmaceutical composition described herein.

In some embodiments, kits of the present disclosure can be provided together with instructions and/or an electronic link to instructions (e.g., a QR code) for administering either or both of (i) fusion polypeptides, nucleic acids encoding fusion polypeptides, or compositions described herein; and (ii) a second therapeutic agent. The instructions will generally include information about the use of the composition for the treatment of any disease or condition described herein. In other embodiments, the instructions include at least one of the following: description of the therapeutic agent; dosage schedule and administration for treatment of any disease or condition described herein or symptoms thereof; precautions; warnings; indications; counter indications; overdose information; adverse reactions; animal pharmacology; clinical studies; and/or references. The instructions may be printed directly on the container (when present), or as a label applied to the container, or as a separate sheet, pamphlet, card, or folder supplied in or with the container.

### EXAMPLES

### Example 1: Materials and Methods

Solubilization of Inclusion Bodies: The inclusion body pellet from 1 L of fermentation was solubilized in 200 mL 50 mM Tris-HCl, 8 M urea, 40 mM DTT, pH 8.0 by homogenization. Solubilization was carried out for 1 h at room temperature. The solubilized material is then clarified by centrifugation (20,000×G for 30 min at 4° C.). The supernatant is decanted and retained. Extracted protein consists of unfolded monomers, with sulfhydryl groups in the reduced state.

Refolding of solubilized inclusion body pellets by dilution: Solubilized fusion polypeptide was added to a refolding solution either rapidly (1-30 min) or slowly (0.5-5 h) to a final concentration of 0.5 to 3.0 mg/mL. The pH of the refolding solution was maintained between 7.3 and 8.5. The refolding solution contained L-arginine (0.5-1.25M) to prevent aggregation. The solution also contained a mixture of reduced and oxidized molecules (1:1 to 6:1 ratio, 0.5 to 8 mM) such as cysteine and cystine or reduced glutathione and oxidized glutathione to initiate disulfide bonding. Refolding was performed in a vessel open to the atmosphere or sparged with air or nitrogen at a temperature between 4-30° C. The clarified, diluted fusion polypeptide was captured on an anion exchange column. The equilibration buffer was 20 mM Tris-HCl, pH 8.0, and the bound fusion polypeptide was eluted with a step-wise gradient to 2 M sodium chloride, in 50 mM Tris-HCl, pH 8. The eluate pool was then passed through a 0.45 μm filter to remove insoluble material.

Ion-Exchange Liquid Chromatography (IEX). Preparative scale IEX was used to separate and quantify charge variants within the refolded fusion polypeptide mixtures isolated from inclusion bodies. Charge variants are multimeric forms, cleaved forms, or deamidated forms of the polypeptides that may exhibit less activity. Charge variant fractionation was performed with a chromatography system equipped with a HiTrap Q XL anion-exchange column. The refolded fusion polypeptide mixture was filtered (0.45 μm filter) immediately before loading to remove particulate material. The ionic strength of the mobile phase should be similar, and the pH identical to that of the refolded fusion polypeptide mixture. The refolded protein mixture was loaded onto the column with a flow rate of 1 mL/min with UV detection at 280 nm. Charge variants were eluted with a step-wise salt gradient (0, 50, 150, 300, 500 mM sodium chloride) and 2 mL fractions were collected in borosilicate glass tubes by a fraction collector. The electropherograms were analyzed and fractions containing single variants are pooled. Fractions and control are concentrated in Vivaspin® 20 centrifugation filter units with 5 kDa MWCO and buffer exchanged with 50 mM Tris-HCl, 150 mM sodium chloride, 1 mM EDTA, pH 8.0. The concentration of the fusion polypeptide was determined via Bradford assay.

Size Exclusion Chromatography (SEC): Preparative scale SEC was used to separate and quantify soluble aggregation of refolded fusion polypeptides after IEX. SEC was performed on a chromatography system equipped with a HiPrep™ Sephacryl S-200 HR column. Approximately 5 mL of concentrated fusion polypeptide was injected into the column at a flow rate of 1 mL/min. The polypeptide was separated by size using a mobile phase of 50 mM Tris-HCl, 1 mM EDTA, 150 mM sodium chloride, pH 8.0 at a flow rate of 2 ml/min with UV detection at 280 nm. As the fusion polypeptide was eluted from the column, a fraction collector was used to collect 2 mL fractions in borosilicate glass tubes. The electropherograms were analyzed and the monomeric fractions of fusion polypeptide were pooled and concentrated.

Desalting/Buffer Exchange Gel Filtration Chromatography (GFC): GFC was used to remove salts and low molecular weight contaminants (urea and beta-mercaptoethanol) from monomeric fusion polypeptide solution in exchange for final formulation buffer. The fusion polypeptide was filtered (0.45 μm filter) immediately before loading to remove particulate material. A HiTrap® Desalting column was pre-equilibrated with final buffer solution. Separations were performed with syringe or chromatography system for larger sample volumes and monitored by following changes in UV absorption and conductivity. The fusion polypeptide was filter sterilized (0.22 μm filter) and the concentration is determined by Bradford assay or via extinction coefficient using Nanodrop spectrophotometer.

Sodium Dodecyl Sulfate-Polyacrylamide Gel Electrophoresis (SDS-PAGE): SDS-PAGE separates polypeptides by mass and provides insight to the size of the purified fusion polypeptides. Fusion polypeptide samples are diluted in Tris-Glycine SDS Sample Buffer with NuPAGE® Reducing Agent, and heat denatured prior to separation by mass on Tris-Glycine precast gels using Tris-Glycine SDS Running Buffer. Polypeptides were stained with SYPRO Ruby Fluorescent Gel Stain and imaged with fluorescent immunoblot imaging system. In some instances, reduced and non-reduced sample were unheated or heat denatured, and profiles were compared to each other and protein molecular weight standards were utilized to confirm the purity and molecular weight of the fusion polypeptides was correct and to determine if there was covalent aggregation indicative of fusion polypeptide misfolding.

Immunoblot: Purified fusion polypeptides were separated by SDS-PAGE (as described above) and transferred to PVDF membrane, blocked, and incubated with primary antibody (e.g. anti-human PRL) that recognizes the fusion polypeptide (SA20-(EAAAK)₃-G129R-hPRL (15-199), anti-pSTAT5, anti-pAKT, anti-pERK1/2, anti-GAPDH), followed by fluorescent secondary antibody (e.g., to detect low amounts of covalent aggregation and degradation, downstream signaling activity). Fluorescent detection of hormone analog moiety (goat anti-mouse Alexa Fluor™ Plus 647) was performed with fluorescent immunoblot imaging system.

Bradford Assay: Coomassie Plus (Bradford) reagent compatible with salts, denaturants, and reducing agents was used to measure the concentration of fusion polypeptides throughout downstream processing. Calibrated sets of external standards containing known amounts of bovine serum albumin (BSA) were used to create calibration curves to determine the concentration of the fusion polypeptides.

### Example 2: Plasmid Construction

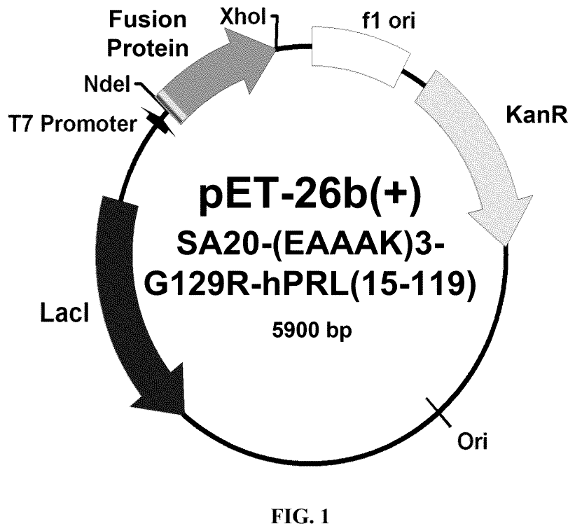

Double stranded DNA encoding SA20-(EAAAK)3-G129R-hPRL (15-199) flanked on the N-terminus by 40 bp homologous region pET-26b(+) vector including an NdeI site and flanked on the C-terminus by a stop codon, XhoI site and 40 bp of homologous regions of pET-26b(+) vector was synthesized as a gBlock (Integrated DNA Technologies). The pET-26b(+) vector (Novagen) was digested with NdeI and XhoI restriction enzymes, separated by electrophoresis and the linearized vector was gel extracted using Qiagen QIAquick Gel Extraction Kit (Qiagen). The gBlock was inserted into the linearized vector by homologous recombination using NEBuilder® HiFi DNA Assembly Kit (New England Biolabs). The assembled pET-26b(+) plasmid product, illustrated in FIG. 1, has an origin of replication which allows it to be propagated in E. coli and can be selectively maintained by the addition of kanamycin due to the expression of a kanamycin resistance gene encoding aminoglycoside phosphotransferase. The expression of fusion polypeptide is under the transcriptional control of the T7 promoter and is strictly prohibited in the absence of Isopropyl β-D-1-thiogalactopyranoside (IPTG) by the LacI repressor.

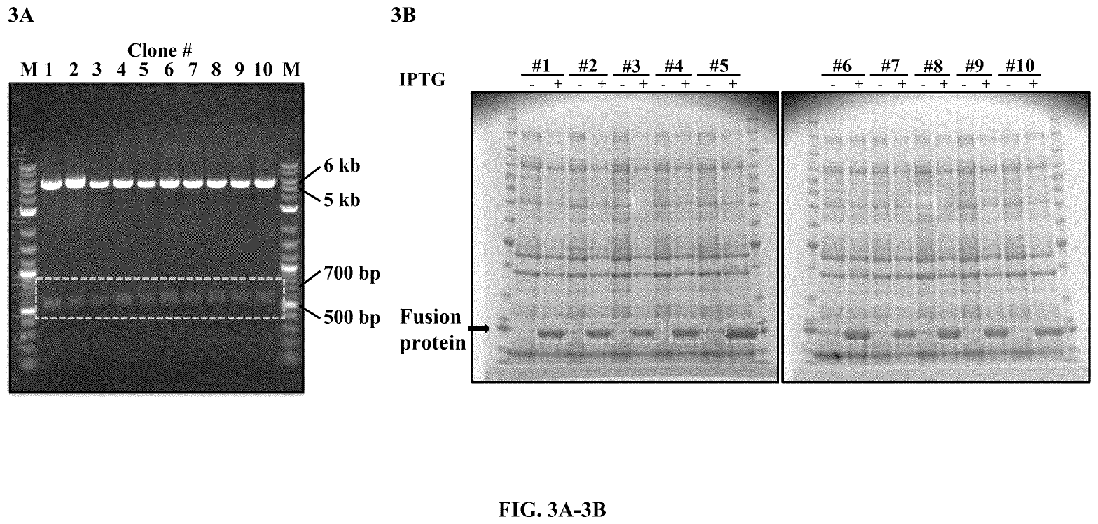

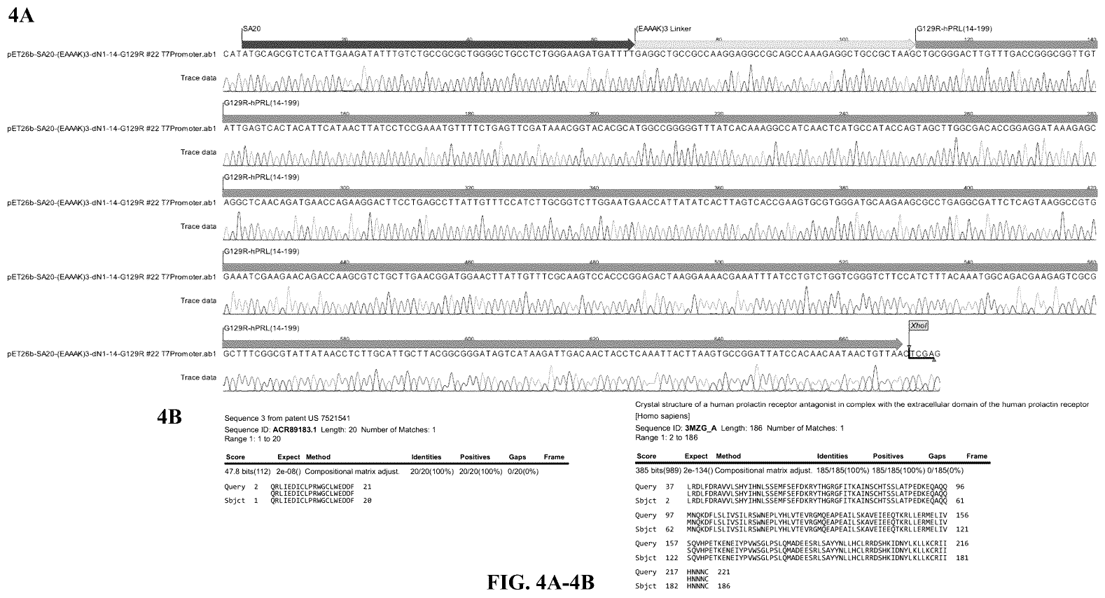

The assembled product was transformed into NEB 5-alpha Competent E. coli, seeded on LB agar plates containing kanamycin and grown overnight at 37° C. Ten individual clones were picked and inoculated into LB broth containing kanamycin and grown overnight in a shaking incubator at 37° C. and 230 rpm. Plasmid DNA was isolated using QIAprep Spin Miniprep Kit (Qiagen) and restriction digested with NdeI and XhoI prior to separation by electrophoresis to check for inserts (FIG. 3A). The DNA sequence of the positive clones was verified by sequencing using T7 promoter primer and T7 terminator primer and standard BigDye™ Terminator 3.1 cycling kit and protocols as known in the art. Sequence information was obtained from and ABI3730 DNA Analyzer and analyzed using CLC Main Workbench (Qiagen) (FIG. 4A) and BLAST searches were used to confirm the identity of the DNA sequences (FIG. 4B).

### Example 3: Production of Fusion Polypeptide

For smaller scale expression, chemically competent BL21 Star™ E. coli expression strain was transformed with pET-26b(+)-SA20-(EAAAK)₃-G129R-hPRL (15-199) construct and selected for on LB agar plates containing kanamycin. Colonies were inoculated into 2 ml of LB medium containing kanamycin and incubated at 37° C., 230 rpm, for 4 h. The culture was split into two tubes, one tube remained uninduced, the second tube was treated with IPTG (1 mM) to induce protein production for an additional 3 h. Uninduced and IPTG-induced cells were pelleted by centrifugation, resuspend in PBS and Laemmli sample buffer and heat-denatured. Lysate was loaded onto 12% polyacrylamide gels and separated by SDS-PAGE prior to staining with SYPRO Ruby Fluorescent Gel Stain to confirm expression of the fusion polypeptide in response to IPTG (FIG. 3B).

For larger scale expression, colonies were inoculated into 100 ml of LB medium containing antibiotic and incubated at 37° C., 230 rpm, for 16 h. The seed liquid was inoculated into Terrific Broth culture medium containing kanamycin (1% v/v). An empirically determined concentration of IPTG (1 mM) was added to the fermentation culture when OD₆₀₀ of the bacterial liquid reached 0.9, and protein production was induced for an additional 4 hrs. IPTG-induced expression of fusion polypeptide in BL21 Star (DE3) E. coli cells transformed with pET-26b(+)-SA20-(EAAAK)₃-G129R-hPRL (14-199) results in the accumulation of large quantities of fusion polypeptide in inclusion bodies. Cell pellets were collected by centrifugation and the cell pellets were stored at −80° C. until inclusion body isolation.

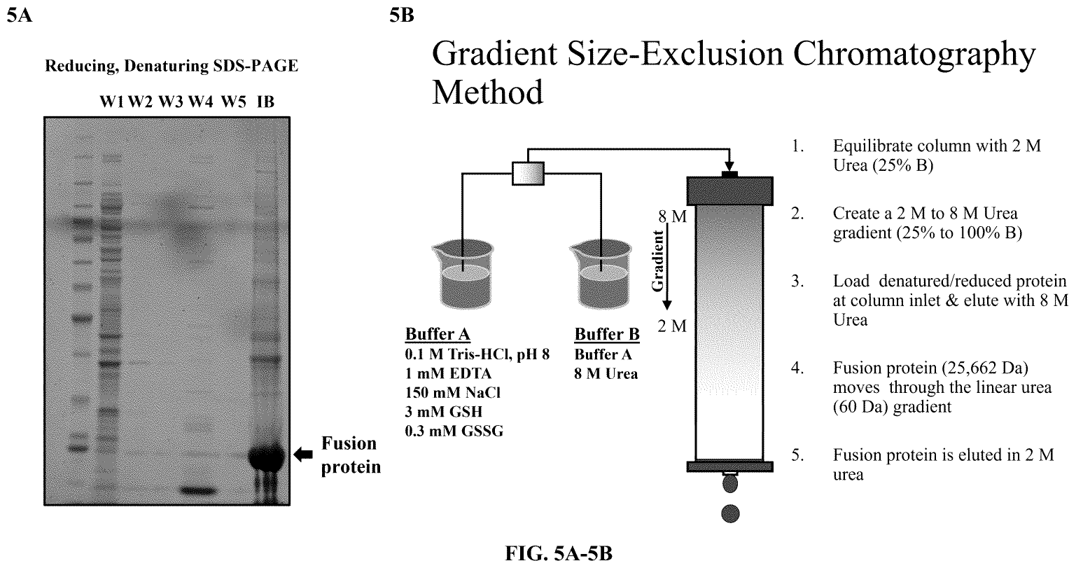

The cell pellet was thawed and resuspended in 10 mL of wash buffer 1 solution (50 mM Tris-HCl, 1 mM EDTA, 1% (v/v) triton, pH 8) per gram wet weight of cells and cell clumps were disrupted with a polytron tissue homogenizer. To aid in the removal of peptidoglycan and outer protein contaminants, 1 mg of lysozyme per gram wet weight of cells was added to the solution and incubated for 30 min at room temperature with intermittent homogenization. The homogenous cell suspension was disrupted by ultrasonic treatment (power is 400-500W, interval is 10 sec on 10 sec off, for 5 minutes on time) in an ice bath. Precipitate was collected by centrifuging (12,000×G, 30 min at 4° C.) and resuspend in wash buffer 1 to extract lipid and membrane-associated proteins. The solution was homogenized to uniformly prior to ultrasonic treatment (power is 400-500W, interval is 10 sec on 10 sec off, for 5 minutes on time) in an ice bath. Precipitate containing the inclusion bodies was collected by centrifugation (12,000×G, 30 min at 4° C.) and the wash process was repeated once more. This process can be repeated as many times as needed until the supernatant is clear. An additional two washes, ultrasonic treatments and centrifugations were performed in wash buffer 2 (50 mM Tris-HCl, 1 mM EDTA, 1 M urea, pH 8) to remove detergent and obtain purified inclusion bodies. Samples from the washes and the purified inclusion bodies were examined by SDS-PAGE to determine the proportions of the protein constituents (FIG. 5A). After purification most of the membrane and cell wall material and other impurities are washed away.

The inclusion body pellet from 1 L of fermentation was solubilized in 200 mL 50 mM Tris-HCl, 8 M urea, 40 mM DTT, pH 8.0 by homogenization. The solubilized material was then clarified by centrifugation (20,000×G for 30 min at 4° C.). The supernatant was decanted and retained. Extracted protein consisted of unfolded monomers, with sulfhydryl groups in the reduced state.

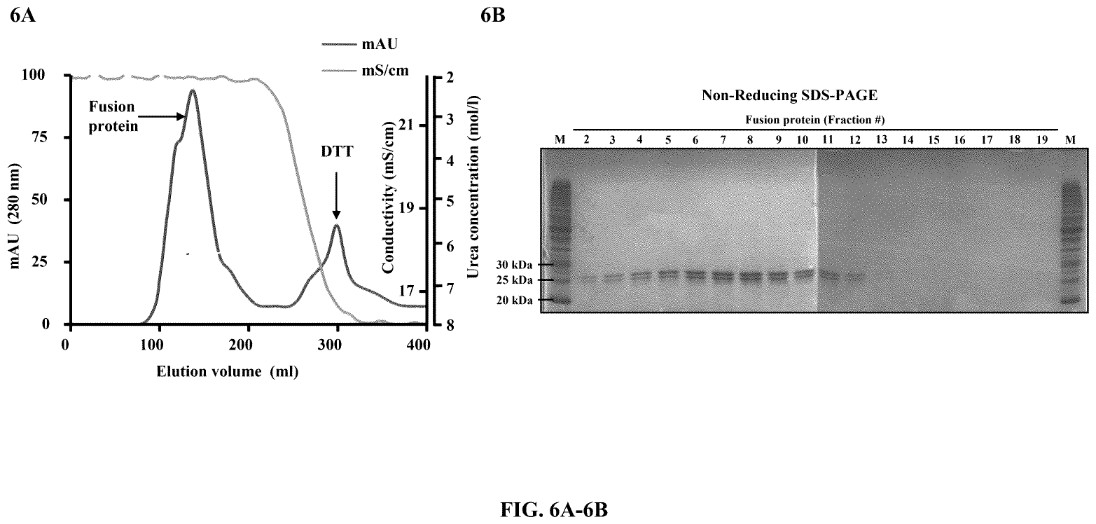

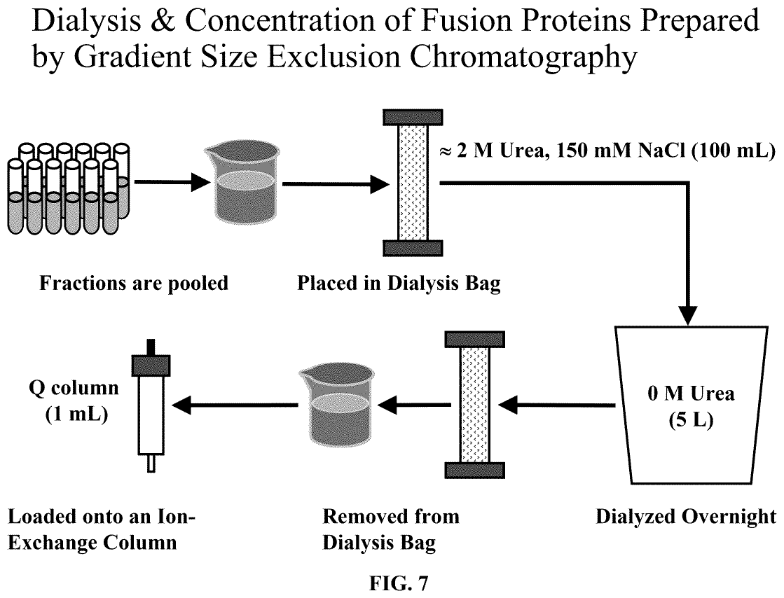

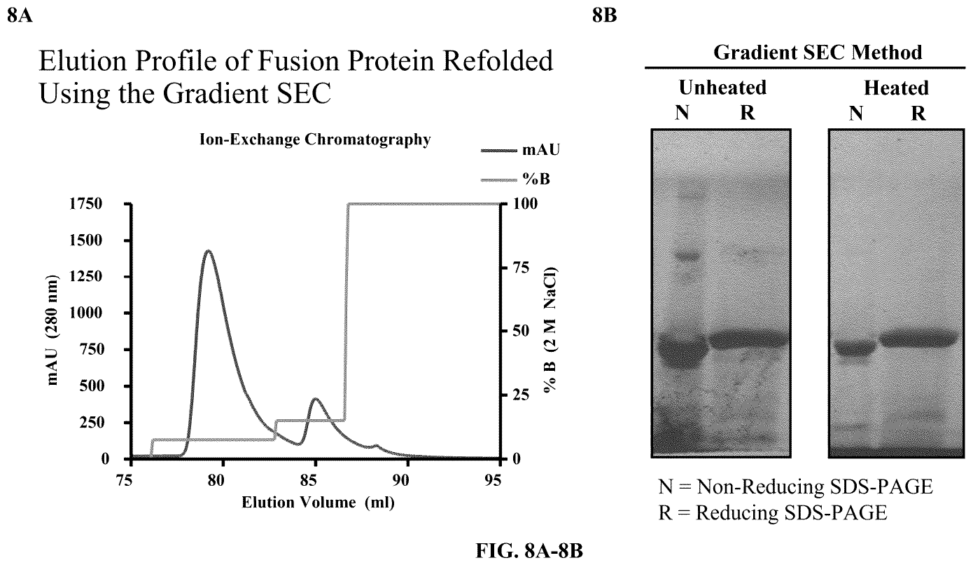

Solubilized fusion polypeptide (25-50 mg/mL) was rapidly refolded by urea gradient size exclusion chromatography (FIG. 5B). A size exclusion chromatography (SEC) column was equilibrated with 1 column of Solution A (100 mM Tris-HCl, 1 mM EDTA, 150 mM sodium chloride, 2 M urea, 3 mM GSH, 0.3 mM GSSG, pH 8). A gradient from 2 M to 8 M urea is created using Solution A in combination with Solution B (100 mM Tris-HCl, 1 mM EDTA, 150 mM sodium chloride, 8 M urea, pH 12) over 0.375 column volume (0% to 100% B). When the gradient was complete, the solubilized fusion polypeptide (0.1-2 mL) was loaded and eluted with 100% Solution B. The fusion polypeptide moves through the linear urea gradient and is eluted in 2 M urea and collected with a fraction collector (FIG. 6A). Non-reducing SDS-PAGE was used to identify fractions for pooling (FIG. 6B). This method tends to prevent aggregation (FIG. 6A) and resulted in the elution of monomeric fusion polypeptide in 2 M urea (FIG. 6B). The monomeric fractions of fusion polypeptide were pooled and dialyzed against 3 L bath of 50 mM Tris-HCl, 1 mM EDTA, pH 8.0, prior to final purification by anion-exchange chromatography (FIG. 7) which resulted in the elution of primarily monomeric fusion polypeptide with minimal dimers or aggregation (FIG. 8A-8B). Recombinant fusion polypeptide was collected and concentrated by ultrafiltration if necessary, using Amicon Ultra filter devices (3000 MWCO; 15 mL). An additional endotoxin removal step may be performed using EndoTrap® affinity column. The final endotoxin content should be below 1 EU/mL at a protein concentration of 1 mg/mL as determined using the Endosafe PTS kit.

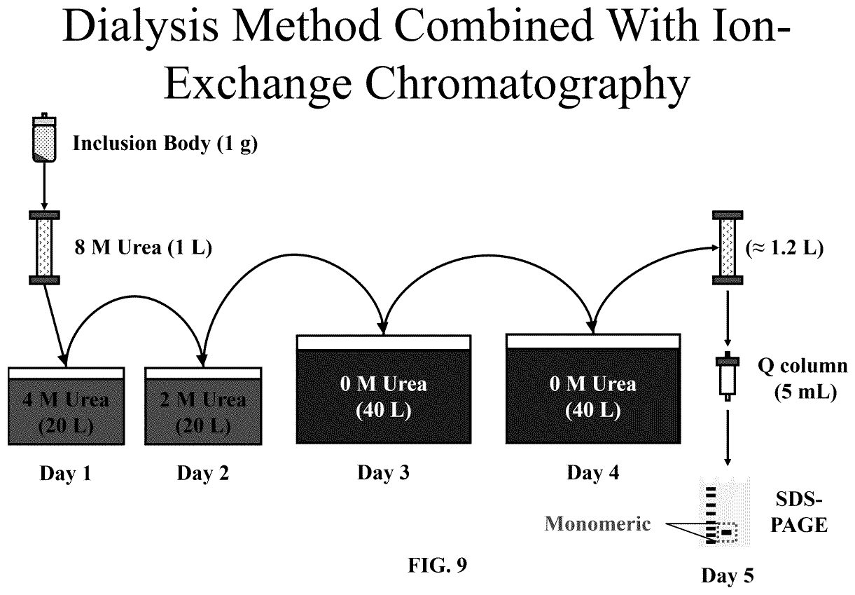

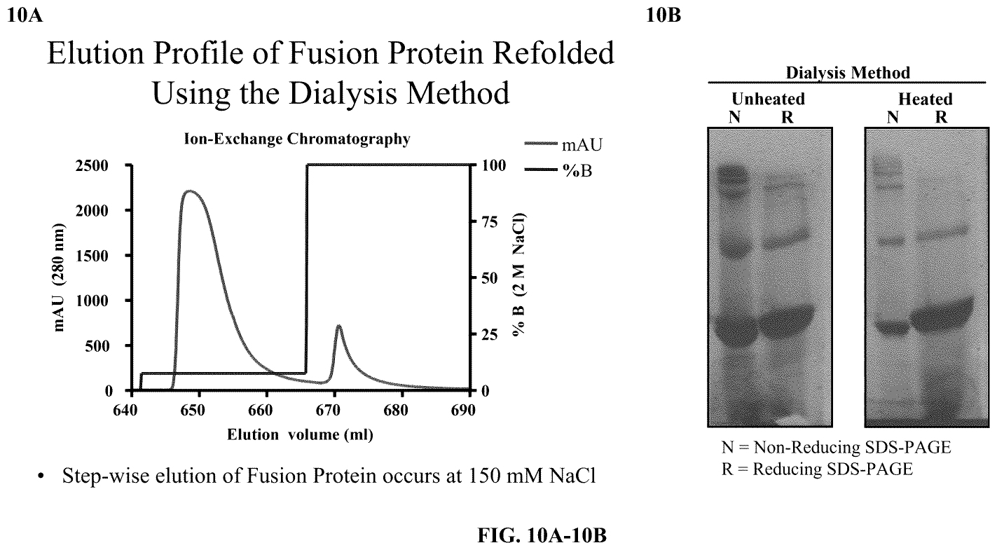

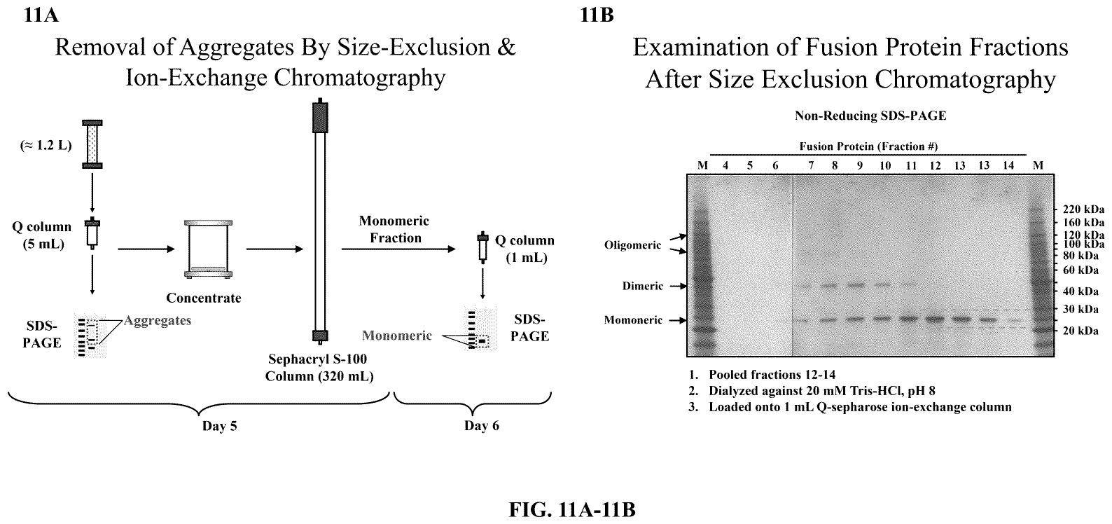

Alternatively, solubilized fusion polypeptide was diluted in 50 mM Tris-HCl, 8 M urea, 40 mM DTT, pH 8.0 (1 g inclusion body pellet/L), transferred to a dialysis bag and dialyzed against four baths of 50 mM Tris-HCl, 1 mM EDTA, pH 8.0 containing 4 M urea, 2 M urea, 0 M urea and 0 M urea (FIG. 9). The fusion polypeptide was passed through a 0.45 μm filter to remove insoluble material was then captured on an anion exchange column. The equilibration buffer was 50 mM Tris-HCl, 1 mM EDTA, pH 8.0, and the bound fusion polypeptide was eluted with a step-wise gradient to 2 M sodium chloride, in 50 mM Tris-HCl, 1 mM EDTA, pH 8 (FIG. 10A). This method tends to yield a significant amount of fusion polypeptide in aggregation (FIG. 10B) which requires protein concentration by ultrafiltration prior loading on a sizing column (FIG. 11A). Size exclusion chromatography was used to collect monomeric fractions (FIG. 11B). Recombinant fusion polypeptide was concentrated by ultrafiltration if necessary, using Amicon Ultra filter devices (3000 MWCO; 15 mL). An additional endotoxin removal step may be performed using EndoTrap® affinity column. The final endotoxin content should be below 1 EU/mL at a protein concentration of 1 mg/mL as determined using the Endosafe PTS kit.

Inclusion body isolated and refolded recombinant protein mixtures are often unstable and aggregate or do not adopt the native conformation necessary for activity. Aggregation impacts the yield, efficacy, and immunogenicity of the final product, so it can be monitored throughout the developmental downstream process. The pH and salt concentration of the refolding buffers can affect aggregation and are altered if necessary to reduce visible aggregation. Molecules, such as L-arginine, may be added to assist with prevention of aggregation during refolding. Physiochemical studies are used to confirm fusion polypeptides are pure and homogenous, soluble, and that their concentration is assessed precisely.

### Example 4: Example of Characterizing Signaling Activity of the Fusion Polypeptide

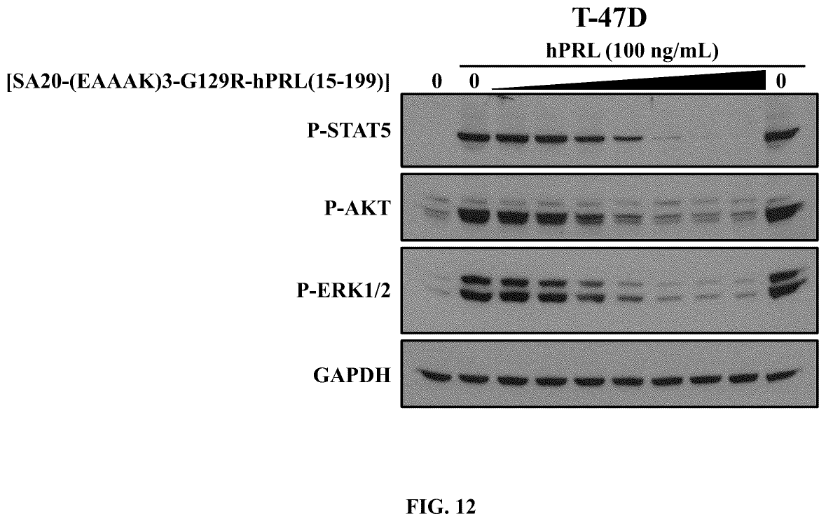

The inhibition of hormone-induced intracellular signaling by an example of a fusion polypeptide of the present disclosure (SA20-(EAAAK)3-G129R-hPRL (15-199)) was assessed by quantitative immunoblotting. Hormone responsive cell lines, T-47D cells (human breast cancer ductal carcinoma) were obtained from the American Type Culture Collection. The cell line was maintained at 37° C. and 5% CO₂ in RPMI 1640 medium supplemented with 10% fetal bovine serum and routinely examined for the presence of mycoplasmas. Cells were trypsinized and resuspended in RPMI medium 1640 supplemented with 10% (v/v) charcoal/dextran-treated FBS (CSS; HyClone, Logan, UT, USA) and 10 μg/ml gentamicin. Approximately 1×10⁶ cells were seeded per well in 12-well tissue culture plates. The cells were grown overnight and depleted for 1 h in RPMI medium 1640 supplemented with 0.5% (v/v) CSS prior to treatment. Cancer cells were treated for 20 min with vehicle or 2.5 nM hormone (hPRL) and increasing doses (0, 5, 15, 25, 50, 70, 100, 300, 500 nM) of fusion polypeptide (SA20-(EAAAK)3-G129R-hPRL (15-199)). Cells were rinsed in PBS, lysed in 100 μL lysis buffer with protease and phosphatase inhibitors, and clarified lysate quantitated by Bradford assay. Cell lysate (30 μg/sample) was separated by SDS-PAGE and immunoblotted overnight with mouse anti-phospho-STAT5A/B (T-47D cells) or mouse anti-phosph-STAT3 antibody (SK-OV3 or HEYA8 cells), mouse anti-phospho-ERK1/2, rabbit anti-phospho-AKT1/2/3 and mouse anti-GAPDH. Immunoblots were probed with secondary antibodies including goat anti-mouse/rabbit secondary antibodies conjugated HRP using a chemiluminescent substate or probed with goat anti-mouse/rabbit secondary antibodies conjugated to fluorescent dye. Total protein loading was fluorescently detected using housekeeping protein, GAPDH. SA20-(EAAAK)3-G129R-hPRL (15-199) was able to competitively inhibit the effects of PRL in dose-dependent manner (FIG. 12).

### Example 5: Example of Characterizing Affinity of the Fusion Polypeptide to Serum Albumin (Prophetic)

The binding of the fusion polypeptides to human serum albumin (HSA) and mouse serum albumin (MSA) is determined by indirect ELISA. 96-well plates are coated with 200 μL of HSA or MSA diluted to 2 mg/mL in PBS at 37° C. for 1 hour. After washing with PBST (PBS+0.1% Tween-20), the wells are blocked with 200 μL of 2% dry milk solution in PBS. Serial dilutions of the fusion polypeptides or unfused hormone analogs in 100 μL of 1% dry milk in PBS are applied to the wells and incubated at 37° C. for 1 hour. Unbound proteins are washed with PBST five times. Detection of bound fusion polypeptide and unfused hormone analog is performed using 50 μL/well mouse anti-PRL antibody (1:1000) and 50 μL/well goat-anti-mouse antibody conjugated to horseradish peroxidase (1:2000). The immune complexes are visualized with 100 μL/well TMB substrate solution incubated for 15 minutes with stirring. The reaction is stopped by adding 100 μL/well 2 N sulfuric acid and the absorbance is measured at 450 nm. Size exclusion chromatography of purified fusion polypeptides and their complexes with HSA or MSA are conducted with a Superdex 200 10/300 GL column at a flow rate of 0.4 ml/min in 100 mM Tris-HCl, pH 8.0, 150 mM sodium chloride.

### Example 6: Example of Characterizing Fusion Polypeptide Pharmacokinetics and Pharmacodynamics (Prophetic)

FVB, NSG or NSG mice humanized for PRL and/or GH at least 8 weeks of age and weighing 22-25 g can be used for pharmacokinetic (PK) studies. Mice are acclimated to a 12 h light: 12 h darkness cycle for at least 1 week prior to the start of the studies and provided food and water ad libitum. Groups of mice (n=12) are administered a single injection (i.v., i.p., or s.c.) of G129R-hPRL (15-199) or equimolar amount of SA20-(EAAAK)3-G129R-hPRL (15-199) in a volume of 10 ml/kg in PBS using 27-gauge needles with a 0.4 mm bore and 13 mm length. Blood drippings (10-50 μl) from six mice are collected after administration at different time points (5, 15, 1, 4, 24, 48 and 72 hours) via tail vein puncture or saphenous vein bleeding. Either plasma or serum is separated from blood cells by centrifugation at 5,000 g for 5 minutes at 4° C., collected, and stored at −80° C. until assay. Concentrations of G129R-hPRL (15-199) and SA20-(EAAAK)3-G129R-hPRL (15-199) are measured using a commercial ELISA kit determined to be insensitive to mouse PRL. G129R-hPRL (15-199) and SA20-(EAAAK)3-G129R-hPRL (15-199) standards (2-200 ng/ml) are prepared in the zero calibrator buffer provided with the kits and used to prepare standard curves. Dilutions of the serum/plasma samples are determined empirically and are made in the zero calibrator buffers. Serum/plasma concentrations are extrapolated from the standard curves prepared for each protein. The serum/plasma concentration (Cs or Cp) verses time (t) profiles are plotted, and noncompartmental methods are used to estimate basic PK parameters. The maximum serum/plasma concentration (Cmax) and the time at which maximum concentration occurs (Tmax) are obtained by inspection of the Cp-t curve. The terminal elimination rate constant (k) is obtained from the terminal slope of the log-linear Cp versus t plots using the last three or four data points within the linear portion of their elimination phases. The terminal half-life (t1/2) is calculated as ln2/k. The area under the serum concentration-time curve (AUC0-t) and area under the first moment of serum concentration-time curve (AUMC0-t) are calculated by numerical integration using the trapezoidal rule. AUC∝ is estimated by dividing the observed concentration at the last time point (Cplast) by k. AUMCx is determined as Cplast×tlast/k+Cplast/k2. The % AUC extrapolated (AUCextrap) is a function of (AUC0-∞-AUC0-t)×100/AUC0-∞ and is an indicator of completeness of the ADME profile. The MRT is calculated by dividing the AUMC0-∞ by AUC0-∞. The plasma clearance (CL) is calculated by dividing the dose by AUC0-∞. The volume of distribution at steady state (Vdss) is obtained from the product of CL and MRT. CL and Vdss values are normalized to animal weight. All calculations can be made using Microsoft Excel or a variety of software products known in the art.

Absorption of G129R-hPRL (15-199) and SA20-(EAAAK)3-G129R-hPRL (15-199) into the bloodstream are expected to differ (if administered i.p. or s.c. rather than i.v.) and reach their maximal observed concentration after different times. The area under the plasma concentration-time curve of SA20-(EAAAK)3-G129R-hPRL (15-199) is expected to increase five-fold or more in comparison with G129R-hPRL (15-199) and to reflect alterations in the distribution and terminal elimination phase of SA20-(EAAAK)3-G129R-hPRL (15-199). The terminal elimination half-life of SA20-(EAAAK)3-G129R-hPRL (15-199) is expected to increase at least five-fold in comparison with G129R-hPRL (15-199) and reflect a reduction in the clearance. The volume of distribution at steady state of SA20-(EAAAK)3-G129R-hPRL (15-199) is expected to be less than that of G129R-hPRL (15-199) due to its maintenance in circulation by binding to serum albumin.

The pharmacodynamics (PD), i.e. inhibitory activity, of fusion polypeptide (e.g. SA20-(EAAAK)3-G129R-hPRL (15-199)) on tumor burden can be examined in comparison with unfused hormone analog (e.g. G129R-hPRL (15-199)) and PBS controls. Subcutaneous cancer cell line derived xenograft tumors are established by injecting 1×10⁶ tumor cells (T-47D, SK-OV-3, or HEYA8) into the flank of six-week-old female PRL and/or GH humanized NSG mice. The mice are sorted into treatment groups (n=10-12) p) when the tumor size reaches 150 mm³ (test day 0). Groups of mice are administered (i.v. or i.p.) SA20-(EAAAK)₃-G129R-hPRL (15-199), an equimolar amount of G129R-hPRL (15-199), or PBS every second or fourth day over a period of 45 days. Tumor size and adverse side effects with respect to weight loss, behavioral changes, respiration, or condition of fur or eyes are monitored in the mice three times per week. The volume of the tumors are measured using a digital caliper and calculated as V (mm³)=π/6×W2×L, where W and L are the width and length of the tumor, respectively. The statistical significance of tumor size differences between treatment groups are assessed by a two-tailed Student's unpaired t-test. Both G129R-hPRL (15-199) and SA20-(EAAAK)3-G129R-hPRL (15-199) are expected to decrease tumor burden in a dosing-dependent manner relative to PBS. The more often they are treated, the greater the extent of tumor growth inhibition is to be expected. The groups receiving SA20-(EAAAK)3-G129R-hPRL (15-199) are expected to have less tumor burden than the groups receiving G129R-hPRL (15-199). Mice receiving SA20-(EAAAK)3-G129R-hPRL (15-199) every fourth day are expected to exhibit more tumor inhibition than mice receiving G129R-hPRL (15-199) every two days (i.e. improved anti-tumor efficacy).

### Example 7: Pharmacokinetic Evaluation of G129R-hPRL (1-199) and SA20-(EAAAK)3-G129R-hPRL (15-199)

To evaluate the pharmacokinetic properties of G129R-hPRL (1-199) compared to fusion polypeptide, SA20-(EAAAK)3-G129R-hPRL (15-199), batches of G129R-hPRL (1-199) and SA20-(EAAAK)3-G129R-hPRL (15-199) were prepared in accordance with the large-scale methods described in Example 3, except solubilized polypeptides were refolded by the dilution refolding method described in Example 1.

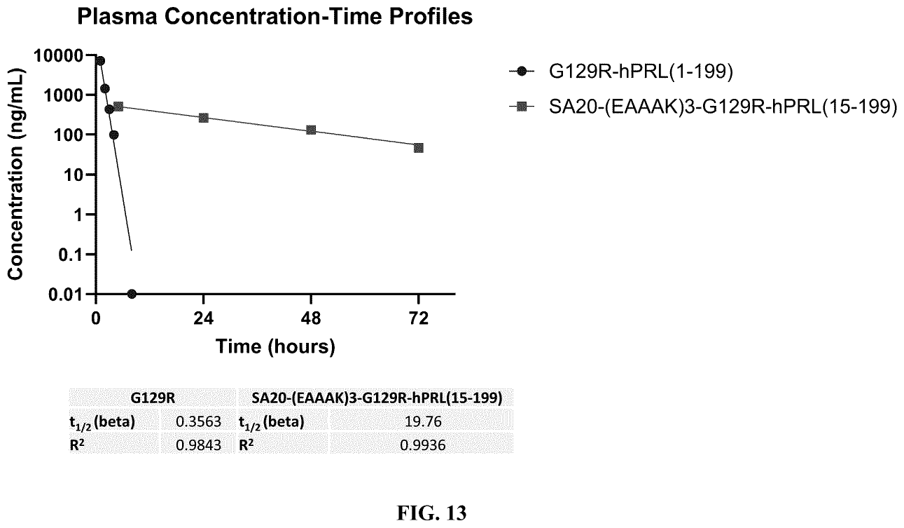

A single molar equivalent dose of either G129R-hPRL (1-199) or SA20-(EAAAK)3-G129R-hPRL (15-199) were administered intraperitoneally (i.p.) to FVB/N mice and the plasma concentration of G129R-hPRL (1-199) or SA20-(EAAAK)3-G129R-hPRL (15-199) was determined at different time points to determine the terminal elimination half-lives (t_{1/2β}) of each polypeptide. The semi-log plot of mean late plasma concentrations over time of G129R-hPRL (1-199) and SA20-(EAAAK)3-G129R-hPRL (15-199) are shown in FIG. 13. Results demonstrated that the terminal elimination half-life of fusion polypeptide, SA20-(EAAAK)₃-G129R-hPRL (15-199), was significantly increased compared to G129R-hPRL (15-199).

1. A fusion polypeptide comprising:
(i) a serum albumin binding polypeptide;
(ii) a polypeptide hormone analog capable of antagonizing its receptor activation; and
(iii) a linker adjoining (i) and (ii),
wherein (i) is N-terminal to the linker and (ii) is C-terminal to the linker.

2. The fusion polypeptide of claim 1, wherein the serum albumin binding polypeptide comprises an amino acid sequence of any one of SEQ ID NOs: 12-46.

3. The fusion polypeptide of claim 1, wherein the polypeptide hormone analog is a prolactin (PRL) analog, optionally wherein the PRL analog comprises an amino acid sequence of any one of SEQ ID NOs: 47-51.

4. (canceled)

5. The fusion polypeptide of claim 1, wherein the polypeptide hormone analog is a growth hormone (GH) analog, optionally wherein the GH analog comprises an amino acid sequence of SEQ ID NO: 54 or SEQ ID NO: 55.

6. (canceled)

7. The fusion polypeptide of claim 1, wherein the polypeptide hormone analog is a placental lactogen (PL) analog, optionally wherein the PL analog comprises an amino acid sequence of SEQ ID NO: 52 or SEQ ID NO: 53.

8. (canceled)

9. The fusion polypeptide of claim 1, wherein the polypeptide hormone analog is N-terminally truncated relative to its parental polypeptide hormone.

10. The fusion polypeptide of claim 1, wherein the linker is a rigid alpha-helical linker, optionally wherein the rigid alpha-helical linker comprises an amino acid sequence of any one of SEQ ID NOs: 56-87.

11. (canceled)

12. The fusion polypeptide of claim 1, further comprising a second linker, wherein the second linker is a flexible linker, optionally wherein the flexible linker comprises an amino acid sequence of any one of SEQ ID NOs: 88-109.

13.-15. (canceled)

16. A nucleic acid encoding a fusion polypeptide of claim 1.

17. (canceled)

18. A host cell comprising the nucleic acid of claim 16 or an expression vector comprising the nucleic acid of claim 16.

19. A method of producing the fusion polypeptide of claim 1 comprising, contacting a cell with a nucleic acid encoding the fusion polypeptide, wherein contacting occurs under conditions sufficient to permit (i) uptake of the nucleic acid by the cell and (ii) translation of the fusion polypeptide.

20. A composition comprising the fusion polypeptide of claim 1, optionally wherein the composition is a pharmaceutical composition, optionally wherein the pharmaceutical composition is an injectable pharmaceutical composition, optionally wherein the pharmaceutical composition is a sustained release or long-lasting pharmaceutical composition.

21.-23. (canceled)

24. A method for treating a disease and/or disorder in a subject comprising administering a therapeutically effective amount of the fusion polypeptide of claim 1 to the subject, optionally wherein the disease or disorder is a cancer.

25.-30. (canceled)

31. The method of claim 24, wherein the fusion polypeptide is administered peripherally, by subcutaneous injection, or intravenously.

32. (canceled)

33. (canceled)

34. The method of claim 24, wherein the fusion polypeptide is administered in combination with a therapeutically effective amount of a second therapeutic agent, optionally wherein the second therapeutic agent is a chemotherapy.

35. (canceled)

36. (canceled)

37. The method of claim 34, wherein administering the fusion polypeptide and the second therapeutic agent in combination synergistically inhibits tumor growth compared to either the fusion polypeptide or the second therapeutic agent when administered as a monotherapy.

38. (canceled)

39. A composition comprising the fusion polypeptide of claim 1 and a second therapeutic agent.

40. A method of maintaining or improving functional activity of a polypeptide hormone analog in a fusion polypeptide, comprising N-terminally truncating the polypeptide hormone analog relative to the parental polypeptide hormone.

41. A kit comprising the fusion polypeptide of claim 1.

42. A method of characterizing the fusion polypeptide of claim 1 comprising assessing one or more of:
(i) signaling activity of the fusion polypeptide;
(ii) affinity of the fusion polypeptide to serum albumin;
(iii) affinity of the fusion polypeptide to its receptor;
(iv) purity of the fusion polypeptide;
(v) yield of the fusion polypeptide;
(vi) efficacy of treating a subject having a cancer; and
(vii) half-life of the fusion polypeptide.
